SEQUENCE LISTING (442 sequences)

SEQ ID NO: 1 - protein, 12 aa, Homo Sapiens
LTITSSLSSDGV

SEQ ID NO: 2 - protein, 9 aa, Homo Sapiens
ERTIPITRE

SEQ ID NO: 3 - protein, 8 aa, Homo Sapiens
FISREFHR

SEQ ID NO: 4 - protein, 14 aa, Homo Sapiens
  MISC_FEATURE (1)..(1): Xaa can be any amino acid
XLTITSSLSSDGVX

SEQ ID NO: 5 - protein, 10 aa, Homo Sapiens
  MISC_FEATURE (1)..(1): Xaa can be any amino acid
XLTITSSLSX

SEQ ID NO: 6 - protein, 11 aa, Homo Sapiens
  MISC_FEATURE (1)..(1): Xaa can be any amino acid
XERTIPITREX

SEQ ID NO: 7 - protein, 10 aa, Homo Sapiens
  MISC_FEATURE (1)..(1): Xaa can be any amino acid
XRTIPITREX

SEQ ID NO: 8 - protein, 10 aa, Homo Sapiens
  MISC_FEATURE (1)..(1): Xaa can be any amino acid
XFISREFHRX

SEQ ID NO: 9 - protein, 10 aa, Homo Sapiens
  MISC_FEATURE (1)..(1): Xaa can be any amino acid
XSREFHRKYX

SEQ ID NO: 10 - protein, 10 aa, Homo Sapiens
  MISC_FEATURE (1)..(1): Xaa can be any amino acid
XHGFISREFX

SEQ ID NO: 11 - protein, 15 aa, Homo Sapiens
  MISC_FEATURE (1)..(1): Xaa can be any amino acid
XHGFISREFHRKYRX

SEQ ID NO: 12 - protein, 11 aa, Homo Sapiens
  MISC_FEATURE (1)..(1): Xaa can be any amino acid
XERTIPITREX

SEQ ID NO: 13 - protein, 23 aa, Homo Sapiens
DPLTITSSLSSDGVLTVNGPRKQ

SEQ ID NO: 14 - protein, 19 aa, Homo Sapiens
LTITSSLSDGVLTVNGPRK

SEQ ID NO: 15 - protein, 11 aa, Homo Sapiens
LTITSSLSDGV

SEQ ID NO: 16 - protein, 13 aa, Homo Sapiens
GPERTIPITREEK

SEQ ID NO: 17 - protein, 12 aa, Homo Sapiens
PERTIPITREEK

SEQ ID NO: 18 - protein, 10 aa, Homo Sapiens
  MISC_FEATURE (1)..(1): Xaa can be any amino acid
XFISREFHRX

SEQ ID NO: 19 - protein, 18 aa, Homo Sapiens
STSLSPFYLRPPSFLRAP

SEQ ID NO: 20 - protein, 13 aa, Homo Sapiens
DRFSVNLDVKHFS

SEQ ID NO: 21 - protein, 8 aa, Homo Sapiens
LTITSSLS

SEQ ID NO: 22 - protein, 8 aa, Homo Sapiens
HGFISREF

SEQ ID NO: 23 - protein, 8 aa, Homo Sapiens
EFHRKYRI

SEQ ID NO: 24 - protein, 8 aa, Homo Sapiens
SREFHRKY

SEQ ID NO: 25 - protein, 8 aa, Homo Sapiens
RTIPITRE

SEQ ID NO: 26 - protein, 4 aa, Homo Sapiens
  MISC_FEATURE (3)..(3): Xaa can be any amino acid
WEXD

SEQ ID NO: 27 - protein, 4 aa, Homo Sapiens
  MISC_FEATURE (3)..(3): Xaa can be any amino acid
DEXD

SEQ ID NO: 28 - protein, 4 aa, Homo Sapiens
  MISC_FEATURE (3)..(3): Xaa can be any amino acid
LEXD

SEQ ID NO: 29 - protein, 4 aa, Homo Sapiens
  MISC_FEATURE (3)..(3): Xaa can be any amino acid
VEXD

SEQ ID NO: 30 - protein, 4 aa, Homo Sapiens
DEVD

SEQ ID NO: 31 - protein, 8 aa, Homo Sapiens
SALSCLSS

SEQ ID NO: 32 - protein, 8 aa, Homo Sapiens
SALSCSLS

SEQ ID NO: 33 - protein, 10 aa, Homo Sapiens
  MISC_FEATURE (1)..(1): Xaa can be any amino acid
XRTIPITREX

SEQ ID NO: 34 - protein, 13 aa, Homo Sapiens
RERAYGEFERTFR

SEQ ID NO: 35 - protein, 13 aa, Homo Sapiens
EIPEEEEIYRTIK

SEQ ID NO: 36 - protein, 13 aa, Homo Sapiens
RERRMGKVYRRIA

SEQ ID NO: 37 - protein, 13 aa, Homo Sapiens
RGIAARQFQRVFV

SEQ ID NO: 38 - protein, 13 aa, Homo Sapiens
RGIATRAFERRFQ

SEQ ID NO: 39 - protein, 13 aa, Homo Sapiens
RGIAARSFEHRFE

SEQ ID NO: 40 - protein, 13 aa, Homo Sapiens
RGIAGRPFEHRFE

SEQ ID NO: 41 - protein, 13 aa, Homo Sapiens
EKFSFREYRGVYR

SEQ ID NO: 42 - protein, 13 aa, Homo Sapiens
REREIGHFRRVVP

SEQ ID NO: 43 - protein, 13 aa, Homo Sapiens
KSISSQARERVIP

SEQ ID NO: 44 - protein, 13 aa, Homo Sapiens
RGIASRAFERRFQ

SEQ ID NO: 45 - protein, 13 aa, Homo Sapiens
RERYYGEIERIIQ

SEQ ID NO: 46 - protein, 13 aa, Homo Sapiens
KEGKYGSFEKKIP

SEQ ID NO: 47 - protein, 13 aa, Homo Sapiens
KGIVFNNFSLNFN

SEQ ID NO: 48 - protein, 13 aa, Homo Sapiens
RERSYGELRRSFY

SEQ ID NO: 49 - protein, 13 aa, Homo Sapiens
RGLAERNFERKFQ

SEQ ID NO: 50 - protein, 13 aa, Homo Sapiens
RGIAARGFVRTFV

SEQ ID NO: 51 - protein, 13 aa, Homo Sapiens
VERAYGTFTRTFS

SEQ ID NO: 52 - protein, 13 aa, Homo Sapiens
SERPSGRFVRELA

SEQ ID NO: 53 - protein, 13 aa, Homo Sapiens
QGLMNQPFSLSFT

SEQ ID NO: 54 - protein, 13 aa, Homo Sapiens
QGIAERNFERKFQ

SEQ ID NO: 55 - protein, 13 aa, Homo Sapiens
KERAYTQFYRAVR

SEQ ID NO: 56 - protein, 13 aa, Homo Sapiens
KERSFMRYYREIP

SEQ ID NO: 57 - protein, 13 aa, Homo Sapiens
RERVTGEVRRRID

SEQ ID NO: 58 - protein, 13 aa, Homo Sapiens
AERPRGVFNRQLV

SEQ ID NO: 59 - protein, 13 aa, Homo Sapiens
REIRYGSFRRSFR

SEQ ID NO: 60 - protein, 13 aa, Homo Sapiens
SEFAYGSFVRTVS

SEQ ID NO: 61 - protein, 13 aa, Homo Sapiens
KERKYGEAKREMR

SEQ ID NO: 62 - protein, 13 aa, Homo Sapiens
QGIAQRAFKLSFR

SEQ ID NO: 63 - protein, 13 aa, Homo Sapiens
IERYYSGYRRVIR

SEQ ID NO: 64 - protein, 13 aa, Homo Sapiens
ERISNFPVSRKIE

SEQ ID NO: 65 - protein, 13 aa, Homo Sapiens
VERYYSGYRRVIR

SEQ ID NO: 66 - protein, 13 aa, Homo Sapiens
DNYMNKNFNYVIS

SEQ ID NO: 67 - protein, 13 aa, Homo Sapiens
SERYSGSMQRMFT

SEQ ID NO: 68 - protein, 13 aa, Homo Sapiens
DERNFESLMRQFD

SEQ ID NO: 69 - protein, 13 aa, Homo Sapiens
SERRYGSFQRTFR

SEQ ID NO: 70 - protein, 13 aa, Homo Sapiens
SERYYGRFERRIA

SEQ ID NO: 71 - protein, 13 aa, Homo Sapiens
RGIAARNFERRFQ

SEQ ID NO: 72 - protein, 13 aa, Homo Sapiens
RGIAARQFQRTFV

SEQ ID NO: 73 - protein, 13 aa, Homo Sapiens
RGIAKRAFERRFQ

SEQ ID NO: 74 - protein, 13 aa, Homo Sapiens
SERRFGSFSRTIT

SEQ ID NO: 75 - protein, 13 aa, Homo Sapiens
SERCVGAFSRTIT

SEQ ID NO: 76 - protein, 13 aa, Homo Sapiens
ATQRPLKIHKVIR

SEQ ID NO: 77 - protein, 13 aa, Homo Sapiens
RGIRKADFQLSFS

SEQ ID NO: 78 - protein, 13 aa, Homo Sapiens
QGLVMQPFSLSFT

SEQ ID NO: 79 - protein, 13 aa, Homo Sapiens
VERSAGKFERAIR

SEQ ID NO: 80 - protein, 12 aa, Homo Sapiens
DQRVDKVYKVVK

SEQ ID NO: 81 - protein, 12 aa, Homo Sapiens
DQRVDKVFKVVR

SEQ ID NO: 82 - protein, 13 aa, Homo Sapiens
QGIAERDFERKFQ

SEQ ID NO: 83 - protein, 13 aa, Homo Sapiens
GRGLTLEGHATLP

SEQ ID NO: 84 - protein, 13 aa, Homo Sapiens
REDRSLFVDAELP

SEQ ID NO: 85 - protein, 13 aa, Homo Sapiens
LRECPDRLERAIT

SEQ ID NO: 86 - protein, 13 aa, Homo Sapiens
TRSQRKTYHRRFR

SEQ ID NO: 87 - protein, 13 aa, Homo Sapiens
IERRYGSFHRRFA

SEQ ID NO: 88 - protein, 13 aa, Homo Sapiens
KESSSGKFKRVIT

SEQ ID NO: 89 - protein, 13 aa, Homo Sapiens
TELKYGAFERTVK

SEQ ID NO: 90 - protein, 13 aa, Homo Sapiens
QGLVRKEFSLTFT

SEQ ID NO: 91 - protein, 13 aa, Homo Sapiens
SEFQYGKFQRVIP

SEQ ID NO: 92 - protein, 13 aa, Homo Sapiens
SEFRYGKFQRVIP

SEQ ID NO: 93 - protein, 13 aa, Homo Sapiens
AERFYGVIERVIP

SEQ ID NO: 94 - protein, 13 aa, Homo Sapiens
SERFYGRFERRIP

SEQ ID NO: 95 - protein, 13 aa, Homo Sapiens
SERYYGRFERRFG

SEQ ID NO: 96 - protein, 13 aa, Homo Sapiens
HGLALRSFARRFE

SEQ ID NO: 97 - protein, 13 aa, Homo Sapiens
QGLAIGNFRQAFK

SEQ ID NO: 98 - protein, 13 aa, Homo Sapiens
RGIAARQFQRCFV

SEQ ID NO: 99 - protein, 13 aa, Homo Sapiens
RERRQGRFVRTVS

SEQ ID NO: 100 - protein, 13 aa, Homo Sapiens
REYEVGDFERQFT

SEQ ID NO: 101 - protein, 13 aa, Homo Sapiens
TEFRYGSFRRVIP

SEQ ID NO: 102 - protein, 13 aa, Homo Sapiens
HGYVSRSFVRKYL

SEQ ID NO: 103 - protein, 13 aa, Homo Sapiens
HGVISRHFIRKYI

SEQ ID NO: 104 - protein, 13 aa, Homo Sapiens
HGYVSRQFSRRYQ

SEQ ID NO: 105 - protein, 13 aa, Homo Sapiens
TKSVYREYNREFL

SEQ ID NO: 106 - protein, 13 aa, Homo Sapiens
HGMIQRHFVRKYT

SEQ ID NO: 107 - protein, 13 aa, Homo Sapiens
HGFITRHFVRRYA

SEQ ID NO: 108 - protein, 13 aa, Homo Sapiens
HGHIMRHFVRRYK

SEQ ID NO: 109 - protein, 13 aa, Homo Sapiens
NGLVERHFVRKYL

SEQ ID NO: 110 - protein, 13 aa, Homo Sapiens
GGYSSRHFLRRFV

SEQ ID NO: 111 - protein, 13 aa, Homo Sapiens
HGHVSRHFVRRYP

SEQ ID NO: 112 - protein, 13 aa, Homo Sapiens
DTFVGRHIVKRFV

SEQ ID NO: 113 - protein, 13 aa, Homo Sapiens
QLCITREFTRSYK

SEQ ID NO: 114 - protein, 13 aa, Homo Sapiens
HGTVAREINRAYK

SEQ ID NO: 115 - protein, 13 aa, Homo Sapiens
HGFSKRSFTRQFT

SEQ ID NO: 116 - protein, 13 aa, Homo Sapiens
GHTLRRSFSRKYS

SEQ ID NO: 117 - protein, 13 aa, Homo Sapiens
QRTVFREYNQEFL

SEQ ID NO: 118 - protein, 13 aa, Homo Sapiens
FGSITRSITRCYR

SEQ ID NO: 119 - protein, 13 aa, Homo Sapiens
FGDVSRNITRCYK

SEQ ID NO: 120 - protein, 13 aa, Homo Sapiens
DNFTKMYFVRKYQ

SEQ ID NO: 121 - protein, 13 aa, Homo Sapiens
YGQVERHFVRKYN

SEQ ID NO: 122 - protein, 13 aa, Homo Sapiens
NISTTQTYSKSIV

SEQ ID NO: 123 - protein, 13 aa, Homo Sapiens
HGASRKSFSRMIL

SEQ ID NO: 124 - protein, 13 aa, Homo Sapiens
YGIVNREVHRTYK

SEQ ID NO: 125 - protein, 13 aa, Homo Sapiens
HGYSKKSFSRVIL

SEQ ID NO: 126 - protein, 13 aa, Homo Sapiens
HGYSKRSFSKMIL

SEQ ID NO: 127 - protein, 13 aa, Homo Sapiens
HGYLKRSFSKMIL

SEQ ID NO: 128 - protein, 13 aa, Homo Sapiens
YGTIESTFKRRFP

SEQ ID NO: 129 - protein, 13 aa, Homo Sapiens
VSYRMSQKVHRKM

SEQ ID NO: 130 - protein, 13 aa, Homo Sapiens
HGYISRCFTRKYT

SEQ ID NO: 131 - protein, 13 aa, Homo Sapiens
HGFVAREFHRRYR

SEQ ID NO: 132 - protein, 13 aa, Homo Sapiens
HGYISREFHRRYR

SEQ ID NO: 133 - protein, 13 aa, Homo Sapiens
HGFVSREFCRTYV

SEQ ID NO: 134 - protein, 13 aa, Homo Sapiens
HGFISRSFTRQYK

SEQ ID NO: 135 - protein, 13 aa, Homo Sapiens
GGIVSKNFTKKIQ

SEQ ID NO: 136 - protein, 18 aa, Homo Sapiens
AEDDIRAAYRNGVLEVRM

SEQ ID NO: 137 - protein, 18 aa, Homo Sapiens
AEDKVAADFRNGVLSVSL

SEQ ID NO: 138 - protein, 23 aa, Homo Sapiens
APESVQSQLTADGHLTIDAKAPE

SEQ ID NO: 139 - protein, 18 aa, Homo Sapiens
DADGITASGSHGVLSIFI

SEQ ID NO: 140 - protein, 20 aa, Homo Sapiens
DADNIKADYANGVLTLTVPK

SEQ ID NO: 141 - protein, 22 aa, Homo Sapiens
DADNIKADYANGVLTLTVPKLK

SEQ ID NO: 142 - protein, 18 aa, Homo Sapiens
DADRIEANFSNGLLTVTL

SEQ ID NO: 143 - protein, 18 aa, Homo Sapiens
DAHSGAATYNNGILEVAF

SEQ ID NO: 144 - protein, 23 aa, Homo Sapiens
DATQARATFSADGILMITVPAPP

SEQ ID NO: 145 - protein, 22 aa, Homo Sapiens
DDSKIDASFLDGVLRITLPKKV

SEQ ID NO: 146 - protein, 20 aa, Homo Sapiens
DDSQLKCRMTDGVLMLEAPV

SEQ ID NO: 147 - protein, 18 aa, Homo Sapiens
DEDDIKATYDKGILTVSV

SEQ ID NO: 148 - protein, 20 aa, Homo Sapiens
DEDDIKATYDKGILTVSVAV

SEQ ID NO: 149 - protein, 22 aa, Homo Sapiens
DEDDIKATYDKGILTVSVAVSE

SEQ ID NO: 150 - protein, 18 aa, Homo Sapiens
DEKAAKANFKNGVLEITL

SEQ ID NO: 151 - protein, 18 aa, Homo Sapiens
DEKLIHASLNNGILSIVM

SEQ ID NO: 152 - protein, 18 aa, Homo Sapiens
DENKVEATYESGLLRVTL

SEQ ID NO: 153 - protein, 18 aa, Homo Sapiens
DESKVNATFRNGVLTVTL

SEQ ID NO: 154 - protein, 23 aa, Homo Sapiens
DFNSIQSSIDAKGRLQVEAGKFN

SEQ ID NO: 155 - protein, 20 aa, Homo Sapiens
DGDNVRADLKNGVLTLTLPK

SEQ ID NO: 156 - protein, 22 aa, Homo Sapiens
DGDNVRADLKNGVLTLTLPKRP

SEQ ID NO: 157 - protein, 18 aa, Homo Sapiens
DIDAVKAKYNNGVLEITI

SEQ ID NO: 158 - protein, 23 aa, Homo Sapiens
DITSVATNLSNDGKLCIEAPKLE

SEQ ID NO: 159 - protein, 22 aa, Homo Sapiens
DKDKVKAELKNGVLLISIPKTK

SEQ ID NO: 160 - protein, 22 aa, Homo Sapiens
DKSQVRAELKNGVLLVSVPKRE

SEQ ID NO: 161 - protein, 23 aa, Homo Sapiens
DLAHIHTVINKEGQMTIDAPKTG

SEQ ID NO: 162 - protein, 20 aa, Homo Sapiens
DLDKISAVCHDGVLKVTVQK

SEQ ID NO: 163 - protein, 22 aa, Homo Sapiens
DLDKISAVCHDGVLKVTVQKLP

SEQ ID NO: 164 - protein, 20 aa, Homo Sapiens
DLDSVKAKMENGVLTLTLHK

SEQ ID NO: 165 - protein, 22 aa, Homo Sapiens
DLDSVKAKMENGVLTLTLHKLS

SEQ ID NO: 166 - protein, 20 aa, Homo Sapiens
DLPSVKSAISEGKLQIEAPK

SEQ ID NO: 167 - protein, 23 aa, Homo Sapiens
DLPSVKSAISNEGKLQIEAPKKT

SEQ ID NO: 168 - protein, 18 aa, Homo Sapiens
DLTKVEADFDHGTLNLRV

SEQ ID NO: 169 - protein, 23 aa, Homo Sapiens
DLTSVKSAISNEGKLQIEAPKKT

SEQ ID NO: 170 - protein, 20 aa, Homo Sapiens
DMDKISAVCRDGVLTVTVEK

SEQ ID NO: 171 - protein, 22 aa, Homo Sapiens
DMDKISAVCRDGVLTVTVEKLP

SEQ ID NO: 172 - protein, 23 aa, Homo Sapiens
DMKTIKSNLDSHGILHIEARKMH

SEQ ID NO: 173 - protein, 18 aa, Homo Sapiens
DPAAVTSALSPEGVLSIQ

SEQ ID NO: 174 - protein, 23 aa, Homo Sapiens
DPATIKSKLDGSGILHISGNKKK

SEQ ID NO: 175 - protein, 18 aa, Homo Sapiens
DPDSAKARYNNGVLEVIL

SEQ ID NO: 176 - protein, 18 aa, Homo Sapiens
DPEKAEAKYENGVLEIRI

SEQ ID NO: 177 - protein, 18 aa, Homo Sapiens
DPGEATAEHVDGVCHVTV

SEQ ID NO: 178 - protein, 18 aa, Homo Sapiens
DPKSAKASYKNGVLEVTF

SEQ ID NO: 179 - protein, 18 aa, Homo Sapiens
DPKSAKASYRNGVLEIKL

SEQ ID NO: 180 - protein, 20 aa, Homo Sapiens
DPLTITSSLSDGVLTVSAPR

SEQ ID NO: 181 - protein, 23 aa, Homo Sapiens
DPLTITSSLSLDGVLTVSAPRKQ

SEQ ID NO: 182 - protein, 23 aa, Homo Sapiens
DPLVITCSLSADGVLTITGPRKV

SEQ ID NO: 183 - protein, 20 aa, Homo Sapiens
DPLVITCSLSDGVLTITGPR

SEQ ID NO: 184 - protein, 20 aa, Homo Sapiens
DPNEVHSTLSDGILTVKAPQ

SEQ ID NO: 185 - protein, 23 aa, Homo Sapiens
DPNEVHSTLSSDGILTVKAPPPL

SEQ ID NO: 186 - protein, 23 aa, Homo Sapiens
DPNEVHSTLSSDGILTVKAPQPL

SEQ ID NO: 187 - protein, 20 aa, Homo Sapiens
DPNEVVSTVSDGVLTLKAPP

SEQ ID NO: 188 - protein, 23 aa, Homo Sapiens
DPNEVVSTVSSDGVLTLKAPPPP

SEQ ID NO: 189 - protein, 18 aa, Homo Sapiens
DPNNVGANLINGLLTLGL

SEQ ID NO: 190 - protein, 18 aa, Homo Sapiens
DPQSAKATYKNGVLEVTF

SEQ ID NO: 191 - protein, 18 aa, Homo Sapiens
DPRSVRIRTRGSLIIVEA

SEQ ID NO: 192 - protein, 18 aa, Homo Sapiens
DPSKAKATYKNGVLSIEL

SEQ ID NO: 193 - protein, 18 aa, Homo Sapiens
DPSTATALYRHGVLIITA

SEQ ID NO: 194 - protein, 22 aa, Homo Sapiens
DPSTVRSHLNSSGVLTITANKL

SEQ ID NO: 195 - protein, 23 aa, Homo Sapiens
DPTKVSSSLSPEGTLTVEAPMPK

SEQ ID NO: 196 - protein, 23 aa, Homo Sapiens
DPTSVTSALREDGSLTIRARRHP

SEQ ID NO: 197 - protein, 23 aa, Homo Sapiens
DPVTVFASLSPEGLLIIEAPQVP

SEQ ID NO: 198 - protein, 23 aa, Homo Sapiens
DPWRVRAALSHDGILNLEAPRGG

SEQ ID NO: 199 - protein, 18 aa, Homo Sapiens
DQANISSSLKNGILTIIL

SEQ ID NO: 200 - protein, 16 aa, Homo Sapiens
DQASISAKYQDGLLTV

SEQ ID NO: 201 - protein, 18 aa, Homo Sapiens
DQKSISARLKNGILTIIL

SEQ ID NO: 202 - protein, 23 aa, Homo Sapiens
DQSALSCSLSADGMLTFCGPKIQ

SEQ ID NO: 203 - protein, 23 aa, Homo Sapiens
DQSAVTCVLSADGMLTFSGSKVQ

SEQ ID NO: 204 - protein, 20 aa, Homo Sapiens
DQSAVTCVLSDGMLTFSGSK

SEQ ID NO: 205 - protein, 20 aa, Homo Sapiens
DSDAIVSTLSDGVLNITVPP

SEQ ID NO: 206 - protein, 23 aa, Homo Sapiens
DSDAIVSTLSEDGVLNITVPPLV

SEQ ID NO: 207 - protein, 17 aa, Homo Sapiens
DSKIDASFLDGVLRITL

SEQ ID NO: 208 - protein, 19 aa, Homo Sapiens
DSKIDASFLDGVLRITLPK

SEQ ID NO: 209 - protein, 18 aa, Homo Sapiens
DTEKIAAKFSKSVLSITL

SEQ ID NO: 210 - protein, 18 aa, Homo Sapiens
DTERILASYQEGVLKLSI

SEQ ID NO: 211 - protein, 20 aa, Homo Sapiens
DTERILASYQEGVLKLSIPV

SEQ ID NO: 212 - protein, 22 aa, Homo Sapiens
DTERILASYQEGVLKLSIPVAE

SEQ ID NO: 213 - protein, 18 aa, Homo Sapiens
DVEKIKAEYKNGVLTIRV

SEQ ID NO: 214 - protein, 23 aa, Homo Sapiens
DVGAVASNLSEDGKLSIEAPKKE

SEQ ID NO: 215 - protein, 23 aa, Homo Sapiens
DVSTVKSHLATRGVLTITASKKA

SEQ ID NO: 216 - protein, 23 aa, Homo Sapiens
DVTHLSSNLSEDGKLLIEVPKVE

SEQ ID NO: 217 - protein, 20 aa, Homo Sapiens
EADKVASTLSDGVLTIKVPK

SEQ ID NO: 218 - protein, 23 aa, Homo Sapiens
EADKVASTLSSDGVLTIKVPKPP

SEQ ID NO: 219 - protein, 23 aa, Homo Sapiens
EADKVTSTLSSDGVLTISVPNPP

SEQ ID NO: 220 - protein, 20 aa, Homo Sapiens
EATAVRSSLSDGMLTVEAPL

SEQ ID NO: 221 - protein, 23 aa, Homo Sapiens
EATAVRSSLSPDGMLTVEAPLPK

SEQ ID NO: 222 - protein, 18 aa, Homo Sapiens
EDDDIEAQYNNGILEVRL

SEQ ID NO: 223 - protein, 18 aa, Homo Sapiens
EDDKVAATFKNGVLTVTL

SEQ ID NO: 224 - protein, 23 aa, Homo Sapiens
EIKDLSAVLCHDGILVVEVKDPV

SEQ ID NO: 225 - protein, 16 aa, Homo Sapiens
EIKSASLKNGLLHVDL

SEQ ID NO: 226 - protein, 18 aa, Homo Sapiens
EINGIEANIKDGVLHLAI

SEQ ID NO: 227 - protein, 20 aa, Homo Sapiens
EKDKIKAELKNGVLFITIPK

SEQ ID NO: 228 - protein, 22 aa, Homo Sapiens
EKDKIKAELKNGVLFITIPKTK

SEQ ID NO: 229 - protein, 22 aa, Homo Sapiens
EKDKVKAELKNGVLYITIPKTK

SEQ ID NO: 230 - protein, 23 aa, Homo Sapiens
ENIHVRGANLVNGLLYIDLERVI

SEQ ID NO: 231 - protein, 23 aa, Homo Sapiens
ENMEVSGATFVNGLLHIDLIRNE

SEQ ID NO: 232 - protein, 16 aa, Homo Sapiens
EQQDISAKYSEGILTV

SEQ ID NO: 233 - protein, 17 aa, Homo Sapiens
ETHLQIQLLNGLLHISY

SEQ ID NO: 234 - protein, 16 aa, Homo Sapiens
EVKAASLANGLLNIDL

SEQ ID NO: 235 - protein, 16 aa, Homo Sapiens
EVQTASLKNGLLHIDL

SEQ ID NO: 236 - protein, 16 aa, Homo Sapiens
EVSGASLKNGLLHIDL

SEQ ID NO: 237 - protein, 16 aa, Homo Sapiens
EVSGATFTNGLLHIDL

SEQ ID NO: 238 - protein, 16 aa, Homo Sapiens
EVSGATFVNGLLHIDL

SEQ ID NO: 239 - protein, 18 aa, Homo Sapiens
EVSGATFVNGLLHIDLIR

SEQ ID NO: 240 - protein, 16 aa, Homo Sapiens
EVTAATLEHGLLHIDL

SEQ ID NO: 241 - protein, 16 aa, Homo Sapiens
EVVGATLKNGLLFVDL

SEQ ID NO: 242 - protein, 18 aa, Homo Sapiens
GVDDIKADYANGVLTLTV

SEQ ID NO: 243 - protein, 17 aa, Homo Sapiens
HGKMNAIFKDGILYITI

SEQ ID NO: 244 - protein, 23 aa, Homo Sapiens
HLDTIRSHLTNSGVLIINVSFSK

SEQ ID NO: 245 - protein, 16 aa, Homo Sapiens
HVRGANLVNGLLYIDL

SEQ ID NO: 246 - protein, 18 aa, Homo Sapiens
HVRGANLVNGLLYIDLER

SEQ ID NO: 247 - protein, 16 aa, Homo Sapiens
HVRGANLVNGLLYIEL

SEQ ID NO: 248 - protein, 18 aa, Homo Sapiens
IPEKAKAKYNNGVLEIRI

SEQ ID NO: 249 - protein, 23 aa, Homo Sapiens
KAEQVVSQLSSDGVLTVSIPKPQ

SEQ ID NO: 250 - protein, 18 aa, Homo Sapiens
KEEGSTAKMENGVLTISL

SEQ ID NO: 251 - protein, 18 aa, Homo Sapiens
KEENASAKFENGVLSVIL

SEQ ID NO: 252 - protein, 20 aa, Homo Sapiens
KEENASAKFENGVLSVILPK

SEQ ID NO: 253 - protein, 20 aa, Homo Sapiens
KIDGIKAEMKNGVLKVTVPK

SEQ ID NO: 254 - protein, 22 aa, Homo Sapiens
KIDGIKAEMKNGVLKVTVPKIK

SEQ ID NO: 255 - protein, 16 aa, Homo Sapiens
KIKGANLVNGLLYIDL

SEQ ID NO: 256 - protein, 20 aa, Homo Sapiens
KPEQIKASMENGVLTVTVPK

SEQ ID NO: 257 - protein, 21 aa, Homo Sapiens
KPEQIKASMENGVLTVTVPKE

SEQ ID NO: 258 - protein, 18 aa, Homo Sapiens
KPETASAGFSDGLLRITV

SEQ ID NO: 259 - protein, 18 aa, Homo Sapiens
KPETATAKFNNGLLDVTV

SEQ ID NO: 260 - protein, 18 aa, Homo Sapiens
KPETAVAKFNNGLLDVTV

SEQ ID NO: 261 - protein, 17 aa, Homo Sapiens
KQHITASFENGLLTITL

SEQ ID NO: 262 - protein, 18 aa, Homo Sapiens
KRDQVTAKYENGVLTIRI

SEQ ID NO: 263 - protein, 16 aa, Homo Sapiens
KVARATLENGLLSVDL

SEQ ID NO: 264 - protein, 21 aa, Homo Sapiens
KVDQVKAGMENGVLTVTVPKN

SEQ ID NO: 265 - protein, 20 aa, Homo Sapiens
KVEEVKAGLENGVLTVTVPK

SEQ ID NO: 266 - protein, 21 aa, Homo Sapiens
KVEEVKAGLENGVLTVTVPKA

SEQ ID NO: 267 - protein, 20 aa, Homo Sapiens
KVEEVKASMENGVLSVTVPK

SEQ ID NO: 268 - protein, 21 aa, Homo Sapiens
KVEEVKASMENGVLSVTVPKV

SEQ ID NO: 269 - protein, 16 aa, Homo Sapiens
KVKKAELSLGLLKLDF

SEQ ID NO: 270 - protein, 16 aa, Homo Sapiens
KVNNAKLEQGLLLVEI

SEQ ID NO: 271 - protein, 16 aa, Homo Sapiens
KVTGATMEHGLLHIDL

SEQ ID NO: 272 - protein, 18 aa, Homo Sapiens
NADGRAELNEDGTLHVFL

SEQ ID NO: 273 - protein, 23 aa, Homo Sapiens
NANEVISDISSDGILTIKAPPPP

SEQ ID NO: 274 - protein, 18 aa, Homo Sapiens
NDKNIQVECENQILTVAV

SEQ ID NO: 275 - protein, 20 aa, Homo Sapiens
NESAIACSLSEGLLTLCCPK

SEQ ID NO: 276 - protein, 23 aa, Homo Sapiens
NESAIACSLSNEGLLTLCCPKTR

SEQ ID NO: 277 - protein, 22 aa, Homo Sapiens
NIEAISAISQDGVLTVTVNKLP

SEQ ID NO: 278 - protein, 22 aa, Homo Sapiens
NKEKITAVCQDGVLTVTVENVP

SEQ ID NO: 279 - protein, 20 aa, Homo Sapiens
NPDGITAAMDKGVLVVTVPK

SEQ ID NO: 280 - protein, 22 aa, Homo Sapiens
NPDGITAAMDKGVLVVTVPKRE

SEQ ID NO: 281 - protein, 23 aa, Homo Sapiens
NPDTVTSSLSSDGLLTIKAPMKA

SEQ ID NO: 282 - protein, 23 aa, Homo Sapiens
NPEQISSTLSTDGVLTVEAPLPQ

SEQ ID NO: 283 - protein, 23 aa, Homo Sapiens
NPEQVVCSLSKNGHLHIQAPRLA

SEQ ID NO: 284 - protein, 20 aa, Homo Sapiens
NPEQVVCSLSNGHLHIQAPR

SEQ ID NO: 285 - protein, 23 aa, Homo Sapiens
NPESIRSSLSKDGVLTVDAPLPA

SEQ ID NO: 286 - protein, 18 aa, Homo Sapiens
NPSAVSAKCGRGLLIVRA

SEQ ID NO: 287 - protein, 16 aa, Homo Sapiens
NVDNAQFENGLLHIDL

SEQ ID NO: 288 - protein, 22 aa, Homo Sapiens
NVEAINAVYQDGVLQVTVEKLP

SEQ ID NO: 289 - protein, 18 aa, Homo Sapiens
QEGDIKARLRNGLLTIAI

SEQ ID NO: 290 - protein, 18 aa, Homo Sapiens
QNDKVQAEYKNGILRLTV

SEQ ID NO: 291 - protein, 18 aa, Homo Sapiens
QNTEVKANYDAGILTLTL

SEQ ID NO: 292 - protein, 18 aa, Homo Sapiens
QNTNVTAEYKDGILNLTL

SEQ ID NO: 293 - protein, 23 aa, Homo Sapiens
QPDTIESHLSDKGVLTICANKTA

SEQ ID NO: 294 - protein, 20 aa, Homo Sapiens
QPDTIESHLSKGVLTICANK

SEQ ID NO: 295 - protein, 16 aa, Homo Sapiens
QVLGAELRNGLLAVDL

SEQ ID NO: 296 - protein, 16 aa, Homo Sapiens
QVREARLRNGLLSIDL

SEQ ID NO: 297 - protein, 18 aa, Homo Sapiens
RIKEIKATYNNGLLQIKV

SEQ ID NO: 298 - protein, 17 aa, Homo Sapiens
RPAGVASLAGGVLTVRF

SEQ ID NO: 299 - protein, 23 aa, Homo Sapiens
RPEQIKSELSNNGVLTVKYEKNQ

SEQ ID NO: 300 - protein, 18 aa, Homo Sapiens
RVEDASAKFENGVLTVEL

SEQ ID NO: 301 - protein, 18 aa, Homo Sapiens
RVESAKAVYKDGVLQIVV

SEQ ID NO: 302 - protein, 16 aa, Homo Sapiens
RVIAAELKNGLLSIDL

SEQ ID NO: 303 - protein, 16 aa, Homo Sapiens
RVLACELRNGLLAIDL

SEQ ID NO: 304 - protein, 16 aa, Homo Sapiens
RVNGADLKNGLLSIDL

SEQ ID NO: 305 - protein, 16 aa, Homo Sapiens
RVSAAELKNGLLSVNL

SEQ ID NO: 306 - protein, 23 aa, Homo Sapiens
SPTAMTCCLTPSGQLWVRGQCVA

SEQ ID NO: 307 - protein, 18 aa, Homo Sapiens
TADGATATVSNGVLTVSL

SEQ ID NO: 308 - protein, 18 aa, Homo Sapiens
TEEGATAQLKNGVLTVTM

SEQ ID NO: 309 - protein, 16 aa, Homo Sapiens
TIVGAKLENGLLIIDL

SEQ ID NO: 310 - protein, 18 aa, Homo Sapiens
TSEAIAASYDAGVLTVRV

SEQ ID NO: 311 - protein, 16 aa, Homo Sapiens
TVTGANLANGLLKIDL

SEQ ID NO: 312 - protein, 18 aa, Homo Sapiens
VPEKAVAKYVDGKLYVKV

SEQ ID NO: 313 - protein, 16 aa, Homo Sapiens
VVVDADLSNGLLSIAL

SEQ ID NO: 314 - protein, 23 aa, Homo Sapiens
YPNDVRSELSSDGILTVKCPPYL

SEQ ID NO: 315 - protein, 5 aa, Homo Sapiens
AAPAS

SEQ ID NO: 316 - protein, 9 aa, Homo Sapiens
AAPASAQAP

SEQ ID NO: 317 - protein, 8 aa, Homo Sapiens
AAQRIAIS

SEQ ID NO: 318 - protein, 9 aa, Homo Sapiens
ADIHVTATD

SEQ ID NO: 319 - protein, 8 aa, Homo Sapiens
AERAIPVS

SEQ ID NO: 320 - protein, 12 aa, Homo Sapiens
AERAIPVSREEK

SEQ ID NO: 321 - protein, 9 aa, Homo Sapiens
AETQAQRIA

SEQ ID NO: 322 - protein, 9 aa, Homo Sapiens
AKPKRIAIN

SEQ ID NO: 323 - protein, 9 aa, Homo Sapiens
AKPRRIEIT

SEQ ID NO: 324 - protein, 8 aa, Homo Sapiens
ALTEKRIP

SEQ ID NO: 325 - protein, 9 aa, Homo Sapiens
ANAKRIAIN

SEQ ID NO: 326 - protein, 8 aa, Homo Sapiens
ASRNIPIR

SEQ ID NO: 327 - protein, 8 aa, Homo Sapiens
ASSGTEQK

SEQ ID NO: 328 - protein, 9 aa, Homo Sapiens
DNGRRIDIH

SEQ ID NO: 329 - protein, 8 aa, Homo Sapiens
DVKSIQIT

SEQ ID NO: 330 - protein, 8 aa, Homo Sapiens
EAQTGPSP

SEQ ID NO: 331 - protein, 12 aa, Homo Sapiens
EAQTGPSPRLGS

SEQ ID NO: 332 - protein, 9 aa, Homo Sapiens
EKPKKIAIE

SEQ ID NO: 333 - protein, 9 aa, Homo Sapiens
EKPKKISIN

SEQ ID NO: 334 - protein, 8 aa, Homo Sapiens
EPKRIAVT

SEQ ID NO: 335 - protein, 11 aa, Homo Sapiens
ERSVYVRQVGP

SEQ ID NO: 336 - protein, 9 aa, Homo Sapiens
ETLIPIAHK

SEQ ID NO: 337 - protein, 9 aa, Homo Sapiens
ETTSSTSIP

SEQ ID NO: 338 - protein, 8 aa, Homo Sapiens
EVKAIQIS

SEQ ID NO: 339 - protein, 8 aa, Homo Sapiens
EVKSVDIS

SEQ ID NO: 340 - protein, 8 aa, Homo Sapiens
FGFLSKFR

SEQ ID NO: 341 - protein, 12 aa, Homo Sapiens
FGFLSKFRCMPE

SEQ ID NO: 342 - protein, 8 aa, Homo Sapiens
FNNELPQD

SEQ ID NO: 343 - protein, 12 aa, Homo Sapiens
FNNELPQDSQEV

SEQ ID NO: 344 - protein, 8 aa, Homo Sapiens
GARPIQIK

SEQ ID NO: 345 - protein, 12 aa, Homo Sapiens
GARPIQIKVINT

SEQ ID NO: 346 - protein, 8 aa, Homo Sapiens
GERLVRVH

SEQ ID NO: 347 - protein, 12 aa, Homo Sapiens
GERLVRVHETGK

SEQ ID NO: 348 - protein, 9 aa, Homo Sapiens
GKNHVKKIE

SEQ ID NO: 349 - protein, 8 aa, Homo Sapiens
GPRMVSIV

SEQ ID NO: 350 - protein, 11 aa, Homo Sapiens
GRSIPIQQAIV

SEQ ID NO: 351 - protein, 11 aa, Homo Sapiens
GRSIPIQQAPV

SEQ ID NO: 352 - protein, 11 aa, Homo Sapiens
GRSVPVKEASM

SEQ ID NO: 353 - protein, 8 aa, Homo Sapiens
HVKKIEVS

SEQ ID NO: 354 - protein, 9 aa, Homo Sapiens
IAAQRIAIS

SEQ ID NO: 355 - protein, 9 aa, Homo Sapiens
IAPQRIAIN

SEQ ID NO: 356 - protein, 9 aa, Homo Sapiens
IEEPKKKIE

SEQ ID NO: 357 - protein, 9 aa, Homo Sapiens
IEVKPMEEE

SEQ ID NO: 358 - protein, 9 aa, Homo Sapiens
KEGEGFEVK

SEQ ID NO: 359 - protein, 8 aa, Homo Sapiens
KEREVTIE

SEQ ID NO: 360 - protein, 12 aa, Homo Sapiens
KEREVTIEQTGE

SEQ ID NO: 361 - protein, 8 aa, Homo Sapiens
KERIIPIK

SEQ ID NO: 362 - protein, 12 aa, Homo Sapiens
KERIIPIKHVGP

SEQ ID NO: 363 - protein, 8 aa, Homo Sapiens
KERIIQIQ

SEQ ID NO: 364 - protein, 12 aa, Homo Sapiens
KERIIQIQQVGP

SEQ ID NO: 365 - protein, 8 aa, Homo Sapiens
KERRIQIQ

SEQ ID NO: 366 - protein, 8 aa, Homo Sapiens
KKDVFQVM

SEQ ID NO: 367 - protein, 9 aa, Homo Sapiens
KKPKRIEIE

SEQ ID NO: 368 - protein, 9 aa, Homo Sapiens
KKPRRIEIN

SEQ ID NO: 369 - protein, 9 aa, Homo Sapiens
KPKPKKRIA

SEQ ID NO: 370 - protein, 8 aa, Homo Sapiens
KPKTIEVK

SEQ ID NO: 371 - protein, 8 aa, Homo Sapiens
KPKTIQVK

SEQ ID NO: 372 - protein, 8 aa, Homo Sapiens
KPKTIQVQ

SEQ ID NO: 373 - protein, 8 aa, Homo Sapiens
KPKTVEVK

SEQ ID NO: 374 - protein, 8 aa, Homo Sapiens
KPRKISVD

SEQ ID NO: 375 - protein, 8 aa, Homo Sapiens
KPRRIEIN

SEQ ID NO: 376 - protein, 8 aa, Homo Sapiens
KPRTIEVK

SEQ ID NO: 377 - protein, 9 aa, Homo Sapiens
KRIEVRSVS

SEQ ID NO: 378 - protein, 9 aa, Homo Sapiens
KRSISVRSG

SEQ ID NO: 379 - protein, 8 aa, Homo Sapiens
KVIDVQIQ

SEQ ID NO: 380 - protein, 8 aa, Homo Sapiens
KVIDVQVQ

SEQ ID NO: 381 - protein, 8 aa, Homo Sapiens
KVTDVEIK

SEQ ID NO: 382 - protein, 9 aa, Homo Sapiens
LKPQKIDIQ

SEQ ID NO: 383 - protein, 9 aa, Homo Sapiens
LKPRKIAIT

SEQ ID NO: 384 - protein, 9 aa, Homo Sapiens
LKPRRIAIG

SEQ ID NO: 385 - protein, 9 aa, Homo Sapiens
LKPRRIEIK

SEQ ID NO: 386 - protein, 9 aa, Homo Sapiens
LKPRTVEIK

SEQ ID NO: 387 - protein, 9 aa, Homo Sapiens
LQPQRIAIG

SEQ ID NO: 388 - protein, 9 aa, Homo Sapiens
MKPRKIEVT

SEQ ID NO: 389 - protein, 9 aa, Homo Sapiens
MKPRRIAIN

SEQ ID NO: 390 - protein, 9 aa, Homo Sapiens
MKPRRIAIS

SEQ ID NO: 391 - protein, 9 aa, Homo Sapiens
MKPRRIEIH

SEQ ID NO: 392 - protein, 9 aa, Homo Sapiens
MQPRKIAIN

SEQ ID NO: 393 - protein, 9 aa, Homo Sapiens
MRPRKIAIE

SEQ ID NO: 394 - protein, 8 aa, Homo Sapiens
NEITIPVT

SEQ ID NO: 395 - protein, 12 aa, Homo Sapiens
NEITIPVTFESR

SEQ ID NO: 396 - protein, 8 aa, Homo Sapiens
NEITLESR

SEQ ID NO: 397 - protein, 8 aa, Homo Sapiens
NERIVQIQ

SEQ ID NO: 398 - protein, 12 aa, Homo Sapiens
NERIVQIQQVGP

SEQ ID NO: 399 - protein, 8 aa, Homo Sapiens
NERSIPIE

SEQ ID NO: 400 - protein, 12 aa, Homo Sapiens
NERSIPIEQVGP

SEQ ID NO: 401 - protein, 8 aa, Homo Sapiens
NEVYISLL

SEQ ID NO: 402 - protein, 12 aa, Homo Sapiens
NEVYISLLPAPP

SEQ ID NO: 403 - protein, 8 aa, Homo Sapiens
NGRRIDIH

SEQ ID NO: 404 - protein, 9 aa, Homo Sapiens
NKPRRIEIN

SEQ ID NO: 405 - protein, 8 aa, Homo Sapiens
PERSIPIT

SEQ ID NO: 406 - protein, 8 aa, Homo Sapiens
PERSVPIS

SEQ ID NO: 407 - protein, 8 aa, Homo Sapiens
PETPIPIS

SEQ ID NO: 408 - protein, 11 aa, Homo Sapiens
PKSIPITIVPK

SEQ ID NO: 409 - protein, 9 aa, Homo Sapiens
PRKISVDRG

SEQ ID NO: 410 - protein, 9 aa, Homo Sapiens
PRRIQVGNA

SEQ ID NO: 411 - protein, 8 aa, Homo Sapiens
QDRPIPVS

SEQ ID NO: 412 - protein, 8 aa, Homo Sapiens
QERIVDIQ

SEQ ID NO: 413 - protein, 12 aa, Homo Sapiens
QERIVDIQQISQ

SEQ ID NO: 414 - protein, 8 aa, Homo Sapiens
QGRSIPIQ

SEQ ID NO: 415 - protein, 9 aa, Homo Sapiens
QNERKIQIK

SEQ ID NO: 416 - protein, 8 aa, Homo Sapiens
QVKAINVY

SEQ ID NO: 417 - protein, 8 aa, Homo Sapiens
RERMIPIE

SEQ ID NO: 418 - protein, 12 aa, Homo Sapiens
RERMIPIEGAGH

SEQ ID NO: 419 - protein, 8 aa, Homo Sapiens
SDRPIPVA

SEQ ID NO: 420 - protein, 8 aa, Homo Sapiens
SEITIPVT

SEQ ID NO: 421 - protein, 8 aa, Homo Sapiens
SERIVQIQ

SEQ ID NO: 422 - protein, 12 aa, Homo Sapiens
SERIVQIQQTGP

SEQ ID NO: 423 - protein, 9 aa, Homo Sapiens
SFSLQFPLS

SEQ ID NO: 424 - protein, 9 aa, Homo Sapiens
SKAKRIAIN

SEQ ID NO: 425 - protein, 9 aa, Homo Sapiens
SKAKRIPIG

SEQ ID NO: 426 - protein, 11 aa, Homo Sapiens
SRSIPINFVAK

SEQ ID NO: 427 - protein, 8 aa, Homo Sapiens
SSRSIPIN

SEQ ID NO: 428 - protein, 8 aa, Homo Sapiens
TEKHIQIR

SEQ ID NO: 429 - protein, 9 aa, Homo Sapiens
TEKHIQIRS

SEQ ID NO: 430 - protein, 8 aa, Homo Sapiens
TERLVQIT

SEQ ID NO: 431 - protein, 12 aa, Homo Sapiens
TERLVQITQTGP

SEQ ID NO: 432 - protein, 9 aa, Homo Sapiens
TRGKQIEVQ

SEQ ID NO: 433 - protein, 8 aa, Homo Sapiens
TVIDVQIQ

SEQ ID NO: 434 - protein, 8 aa, Homo Sapiens
TYSRVLVK

SEQ ID NO: 435 - protein, 12 aa, Homo Sapiens
TYSRVLVKDGVR

SEQ ID NO: 436 - protein, 7 aa, Homo Sapiens
VAELKID

SEQ ID NO: 437 - protein, 7 aa, Homo Sapiens
VAFNKGL

SEQ ID NO: 438 - protein, 12 aa, Homo Sapiens
VEREIEIEPTGN

SEQ ID NO: 439 - protein, 8 aa, Homo Sapiens
VQQTFRTE

SEQ ID NO: 440 - protein, 11 aa, Homo Sapiens
VQQTFRTEIKI

SEQ ID NO: 441 - protein, 11 aa, Homo Sapiens
VRALPIHTSAG

SEQ ID NO: 442 - protein, 9 aa, Homo Sapiens
VTARPAPGD

(19) United States
(12) Patent Application Publication
(10) Pub. No.: US 2008/0280824 A1
(43) Pub. Date: Nov. 13, 2008

(54) COMPOSITIONS AND METHODS FOR TREATMENT OF CANCER OR NEURODEGENERATIVE DISEASE WITH PEPTIDE BASED MICROTUBULE STABILIZERS OR INHIBITORS

(75) Inventors: Joy G. Ghosh, Seattle, WA (US); John I. Clark, Seattle, WA (US); Scott Andrew Houck, Woodinville, WA (US)

Correspondence Address: WOODCOCK WASHBURN LLP, CIRA CENTRE, 12TH FLOOR, 2929 ARCH STREET, PHILADELPHIA, PA, 19104-2891, US

(73) Assignee: University of Washington, Seattle, WA (US)

(21) Appl. No.: 11/745,360

(22) Filed: May 7, 2007

(51) Int. Cl. A61K 38/00; A61P 25/00; C12Q 1/02

(52) U.S. Cl. 514/12; 514/13; 514/15; 514/16; 514/17; 514/18; 514/19; 435/29

(57) ABSTRACT

Small molecular weight molecules are provided including, but not limited to, peptides, peptide analogs and peptide mimetics that can interact with microtubules to promote their assembly or prevent their disassembly and can interrupt mitosis, prevent cell division, and trigger apoptosis. methods for the prevention or treatment of neoplastic disease in a mammalian subject are provided utilizing peptides, peptide analogs and peptide mimetics, or utilizing nucleic acids encoding the peptides.

### STATEMENT OF GOVERNMENT SUPPORT

This invention was made by government support by Grant No. EY04542 from National Eye Institute of The National Institutes of Health. The Government has certain rights in this invention.

### FIELD

The invention is directed to small molecular weight molecules including, but not limited to, peptides, peptide analogs and peptide mimetics that can interact with microtubules to promote their assembly or prevent their disassembly and can interrupt mitosis, prevent cell division, and trigger apoptosis. The invention relates to methods for the prevention or treatment of neoplastic disease in a mammalian subject by administering peptides, peptide analogs and peptide mimetics.

### BACKGROUND

Microtubule assembly is critical for cell division, neuronal transport, and cell signaling, thus making them excellent therapeutic targets for cancer and neurodegenerative diseases. Molecular chaperones are endogenous molecules in the body that play critical roles in the normal folding, processing, organization, and degradation of cellular proteins including various filament proteins. Wang et al., Circ Res 93:998-1005, 2003; Minami et al., Acta Neuropathol (Berl) 105:549-54, 2003; Horwitz, J., Acad Sci USA 89:10449-53, 1992. Low molecular weight (<43 kDa) chaperones known as small heat shock proteins (sHSPs) are important for the normal organization and stability of cytoskeleton filament networks including microtubules. Quinlan, R., Prog Mol Subcell Biol 28:219-33, 2002; Head et al., Neuroreport 11:361-5, 2000; Djabali et al., Exp Cell Res 253: 649-62, 1999; Muchowski et al., Invest Opthalmol V is Sci 40:951-8, 1999; Liang, P. and MacRae, T. H., J Cell Sci 110 (Pt 13) 1431-40, 1997; Prescott et al., Ophthalmic Res 28: Suppl 1: 58-61, 1996; Gopalakrishnan et al., Trans Kans Acad Sci 96:7-12, 1993; Tomokane et al., Am J Pathol 138:875-85, 1991; Lieska et al., Biochim Biophys Acta 626:136-53, 1980; Bennardini et al., Circ Res 71:288-94, 1992; Wisniewski, T. and Goldman, J. E., Neurochem Res 23:385-92; Launay et al., Exp Cell Res, 2006; Maglara et al., J Physiol 548:837-46, 2003. The archetype of sHSPs, human αB crystallin, and a homologous protein sHSP27 interact directly with tubulin to promote normal assembly into microtubules and protect the structural stability of microtubules in response to stress. Liang, P. and MacRae, T. H., J Cell Sci 110 (Pt 13) 1431-40, 1997; Fujita et al., J Cell Sci 117:1719-26, 2004; Sakurai et al., Faseb J, 2005; Xi et al., Faseb J 20:846-57, 2006; Day et al, Cell Stress Chaperones 8:183-93, 2003; Atomi et al., Biol Sci Space 15:206-7, 2001; Arai, H. and Atomi, Y., Cell Struct Funct 22:539-44, 1997; Kato et al., J Biol Chem 271:26989-94, 1996; Bauer, N. G. and Richter-Landsberg, C., J Mol Neurosci 29:153-68, 2006. However, a recent report suggests that sHSPs including a crystallin inhibit microtubule assembly at high concentrations. Mitra et al., Proteins, 2007. High levels of the molecular chaperones αB crystallin, sHSP27, and other sHSPs in patients with neurodegenerative diseases (Wilhelmus et al., Neuropathl Appl Neurobiol 32:119-30, 2006; Renkawek et al., Neuroreport 10:2273-6, 1999) and the increased expression of αB crystallin and sHSP25 in transgenic mouse models for fALS, Parkinson's disease (PD), dentato-rubral pallido-luysian atrophy (DRPLA), and Huntington's disease (HD are the basis for mechanisms of sHSP protection in neurodegenerative diseases. Wang et al., Neurobiol Aging, 2007. Recently, novel protein pin arrays and mutagenesis characterized five interactive domains in human αB crystallin that mediated interactions with selected substrate proteins including lens crystallins and filament proteins. Ghosh, J. G. and Clark, J. I., Protein Sci 14:684-95, 2005; Ghosh, et al., Biochemistry 44:14854-69, 2005; Ghosh et al., Cell Stress Chaperones 11:187-97.

Peptides that interact with microtubules to prevent their disassembly can interrupt mitosis, preventing cell division, and triggering apoptosis. Modulation of microtubule assembly is of great interest in the development of new cancer treatments. Two of the most important anti-cancer drugs today, Paclitaxel and Docetaxel are examples of molecules that stabilize microtubules and prevent their disassembly. However, undesirable side effects including drug resistance limit the effectiveness of many current anti-cancer agents. A need exists in the art for anti cancer therapies that are effect and have fewer undesirable side effects or adverse reactions.

### SUMMARY

The present invention relates to methods for the prevention or treatment of neoplastic disease in a mammalian subject. Peptides, peptide analogs and peptide mimetics are provided that interact with microtubules to prevent their disassembly and can interrupt mitosis, prevent cell division, and trigger apoptosis. Modulation of microtubule assembly is a cellular mechanism which can be regulated leading to the development of treatment for cancer and neoplastic diseases. The αB crystallin peptide LTITSSLSSDGV, peptide ERTIPITRE, and peptide FISREFHR, and analogs and mimetics thereof, disrupt tubulin [symbol] microtubule dynamics and have the potential to be developed into safe new therapeutics for neoplastic diseases, cancer, solid tumors and neurodegenerative diseases. Peptides, peptide analogs, or peptide-mimetics are provided which interact with tubulin to promote microtubule assembly, inhibit microtubule disassembly, or decrease thermal aggregation of tubulin, and therein induce apoptosis in cells in a mammalian subject.

A method for treating a neoplastic disease in a mammalian subject is provided which comprises administering a polypeptide to the subject in need thereof, wherein the polypeptide is X₁-LTITSSLSSDGV-X₂, or a functional variant or mimetic thereof, wherein each X₁ and X₂ independently of one another represents any amino acid sequence of n amino acids, n varying from 0 to 50, and n being identical or different in X₁ and X₂, in an amount effective to reduce or eliminate the neoplastic disease in the subject. The functional variant or mimetic can comprise a conservative amino acid substitution or peptide mimetic substitution. The functional variant can comprise about 70% or greater amino acid sequence identity to X₁-LTITSSLSSDGV-X₂. The functional variant can have about 70% or greater amino acid sequence identity to X₁-LTITSSLS-X₂. In a detailed aspect, the functional variant is X₁-LTITSSLS-X₂. In a further detailed aspect, the functional variant is an L-enantiomer or D-enantiomer of one or more amino acids. The neoplastic disease includes, but is not limited to, a solid tumor, carcinoma, sarcoma, lymphoma, or leukemia.

A method for treating a neoplastic disease in a mammalian subject is provided which comprises administering a polypeptide to the subject in need thereof, wherein the polypeptide is X₁-ERTIPITRE-X₂, or a functional variant or mimetic thereof, wherein each X₁ and X₂ independently of one another represents any amino acid sequence of n amino acids, n varying from 0 to 50, and n being identical or different in X₁ and X₂, in an amount effective to reduce or eliminate the neoplastic disease in the subject. The functional variant or mimetic can comprise a conservative amino acid substitution or peptide mimetic substitution. The functional variant can comprise about 70% or greater amino acid sequence identity to X₁-ERTIPITRE-X₂. The functional variant can have about 70% or greater amino acid sequence identity to X₁-RTIPITRE-X₂. In a detailed aspect, the functional variant is X₁-RTIPITRE-X₂. In a further detailed aspect, the functional variant is an L-enantiomer or D-enantiomer of one or more amino acids. The neoplastic disease includes, but is not limited to, a solid tumor, carcinoma, sarcoma, lymphoma, or leukemia.

A method for treating a neoplastic disease in a mammalian subject is provided which comprises administering a polypeptide to the subject in need thereof, wherein the polypeptide is X₁-FISREFHR-X₂, or a functional variant or mimetic thereof, wherein each X₁ and X₂ independently of one another represents any amino acid sequence of n amino acids, n varying from 0 to 50, and n being identical or different in X₁ and X₂, in an amount effective to reduce or eliminate the neoplastic disease in the subject. The functional variant or mimetic can comprise a conservative amino acid substitution or peptide mimetic substitution. The functional variant can comprise about 70% or greater amino acid sequence identity to X₁-FISREFHR-X₂. The functional variant can have about 70% or greater amino acid sequence identity to X₁-SREFHRKY-X₂, X₁-HGFISREF-X₂, or X₁-HGFISREFHRKYR-X₂. In a detailed aspect, the functional variant is X₁-SREFHRKY-X₂, X₁-HGFISREF-X₂, or X₁-HGFISREFHRKYR-X₂. In a further detailed aspect, the functional variant is an L-enantiomer or D-enantiomer of one or more amino acids. The neoplastic disease includes, but is not limited to, a solid tumor, carcinoma, sarcoma, lymphoma, or leukemia.

A method for treating a neurodegenerative disease in a mammalian subject comprising administering a polypeptide to the subject in need thereof, wherein the polypeptide is X₁-LTITSSLSSDGV-X₂, or a functional variant or mimetic thereof, wherein each X₁ and X₂ independently of one another represents any amino acid sequence of n amino acids, n varying from 0 to 50, and n being identical or different in X₁ and X₂, or the polypeptide is X₃-ERTIPITRE-X₄, or a functional variant or mimetic thereof, wherein each X₃ and X₄ independently of one another represents any amino acid sequence of n amino acids, n varying from 0 to 50, and n being identical or different in X₃ and X₄, in an amount effective to reduce or eliminate the neurodegenerative disease in the subject. The functional variant or mimetic can comprise a conservative amino acid substitution or peptide mimetic substitution. The functional variant can comprise about 70% or greater amino acid sequence identity to X₁-LTITSSLSSDGV-X₂. The functional variant can have about 70% or greater amino acid sequence identity to X₁-LTITSSLS-X₂. In a detailed aspect, the functional variant is X₁-LTITSSLS-X₂. The functional variant can comprise about 70% or greater amino acid sequence identity to X₁-ERTIPITRE-X₂. The functional variant can have about 70% or greater amino acid sequence identity to X₁-RTIPITRE-X₂. In a detailed aspect, the functional variant is X₁-RTIPITRE-X₂. In a further detailed aspect, the functional variant is an L-enantiomer or D-enantiomer of one or more amino acids. The neurodegenerative disease includes, but is not limited to, taupathy, Alzheimer's disease, motor neuron disease, hypoparathyroidism-retardation-dysmorphic syndrome, Parkinson's disease, schizophrenia, or Lewy body disease.

A method for inducing apoptosis of a cell in a mammalian subject is provided which comprises administering a polypeptide to the subject in need thereof, wherein the polypeptide is X₁-LTITSSLSSDGV-X₂, or a functional variant or mimetic thereof, wherein each X₁ and X₂ independently of one another represents any amino acid sequence of n amino acids, n varying from 0 to 50, and n being identical or different in X₁ and X₂, or the polypeptide is X₃-ERTIPITRE-X₄, or a functional variant or mimetic thereof, wherein each X₃ and X₄ independently of one another represents any amino acid sequence of n amino acids, n varying from 0 to 50, and n being identical or different in X₃ and X₄, in an amount effective to induce apoptosis and to reduce or eliminate a disease in the subject. The functional variant or mimetic can comprise a conservative amino acid substitution or peptide mimetic substitution. The functional variant can comprise about 70% or greater amino acid sequence identity to X₁-LTITSSLSSDGV-X₂. The functional variant can have about 70% or greater amino acid sequence identity to X₁-LTITSSLS-X₂. In a detailed aspect, the functional variant is X₁-LTITSSLS-X₂. The functional variant can comprise about 70% or greater amino acid sequence identity to X₁-ERTIPITRE-X₂. The functional variant can have about 70% or greater amino acid sequence identity to X₁-RTIPITRE-X₂. In a detailed aspect, the functional variant is X₁-RTIPITRE-X₂. In a further detailed aspect, the functional variant is an L-enantiomer or D-enantiomer of one or more amino acids. The neoplastic disease includes, but is not limited to, a solid tumor, carcinoma, sarcoma, lymphoma, or leukemia.

Libraries of Intellipeptides, or polypeptides, peptide analogs, or peptide-mimetics are provided herein which interact with tubulin to promote microtubule assembly, inhibit microtubule disassembly, or decrease thermal aggregation of tubulin, and therein induce apoptosis in cells in a mammalian subject. Such libraries include both peptide libraries and libraries of nucleic acid constructs capable of expressing Intellipeptides. In one embodiment, a library of the present invention consists of sequences related to i) DPLTITSSLSSDGVLTVNGPRKQ; ii) LTITSSLSDGVLTVNGPRK; iii) LTITSSLSDGV; iv) GPERTIPITREEK; v) PERTIPITREEK; vi) ERTIPITRE; or functional variants or peptide mimetics thereof. In a particular embodiment, a library of the invention consists of two or more Intellipeptides or encoding sequences, including, e.g., the sequences provided in FIG. 7 (LTITSSLSDGV), FIG. 8 (ERTIPITRE), or FIG. 9. (FISREFHR).

An in vivo method of screening for a modulator of microtubule assembly or disassembly activity is provided which comprises contacting a cell with a test compound encoding a polypeptide X₁-LTITSSLSSDGV-X₂, or a functional variant or mimetic thereof, wherein each X₁ and X₂ independently of one another represents any amino acid sequence of n amino acids, n varying from 0 to 50, and n being identical or different in X₁ and X₂, or a polypeptide X₃-ERTIPITRE-X₄, or a functional variant or mimetic thereof, wherein each X₃ and X₄ independently of one another represents any amino acid sequence of n amino acids, n varying from 0 to 50, and n being identical or different in X₃ and X₄, or the polypeptide is X₅-FISREFHR-X₆, or a functional variant or mimetic thereof, wherein each X₅ and X₆ independently of one another represents any amino acid sequence of n amino acids, n varying from 0 to 50, and n being identical or different in X₁ and X₂, and detecting an interaction of the test compound with tubulin to promote microtubule assembly, inhibit microtubule disassembly, or decrease thermal aggregation of tubulin in the cell or cell line. In a further aspect, the detecting step further comprises detecting apoptosis in the cell. The functional variant or mimetic can comprise a conservative amino acid substitution or peptide mimetic substitution. The functional variant can comprise about 70% or greater amino acid sequence identity to X₁-LTITSSLSSDGV-X₂. The functional variant can have about 70% or greater amino acid sequence identity to X₁-LTITSSLS-X₂. In a detailed aspect, the functional variant is X₁-LTITSSLS-X₂. The functional variant can comprise about 70% or greater amino acid sequence identity to X₁-ERTIPITRE-X₂. The functional variant can have about 70% or greater amino acid sequence identity to X₁-RTIPITRE-X₂. In a detailed aspect, the functional variant is X₁-RTIPITRE-X₂. In a further detailed aspect, the functional variant is an L-enantiomer or D-enantiomer of one or more amino acids.

### BRIEF DESCRIPTION OF THE DRAWINGS

FIG. 1 shows an effect of αB crystallin peptides on microtubule assembly.

FIG. 2 shows surface locations of the interactive sequences in αB crystallin for subunit-subunit interactions, chaperone activity, and interactions with filaments and tubulin.

FIG. 3 shows an effect of synthetic αB crystallin peptides on microtubule assembly, disassembly, and tubulin aggregation.

FIG. 4 shows an effect of mutations in three αB crystallin interactive domains on microtubule assembly, disassembly, and tubulin aggregation.

FIG. 5 shows an effect of αB crystallin concentration on microtubule assembly.

FIG. 6 shows a model of the tubulin interactive sequences in the human αB crystallin complex and their importance in the assembly of microtubules.

FIG. 7 shows the amino acid sequence of a series of peptides derived from the sequence FISREFHR.

FIG. 8 shows the amino acid sequence of a series of peptides derived from the sequence LTITSSLSSDGV.

FIG. 9 shows the amino acid sequence of a series of peptides derived from the sequence ERTIPITRE.

### DETAILED DESCRIPTION

The present invention relates to methods for the prevention or treatment of neoplastic disease in a mammalian subject. The present invention further provides polypeptides, peptide analogs and peptide mimetics that promote or inhibit microtubule assembly, prevent protein misfolding, abnormal folding, and/or aggregation and are useful in a variety of therapeutic and manufacturing applications, including the treatment of diseases and disorders associated with microtubule assembly and the ability to induce apoptosis in cells in the mammalian subject, e.g., providing a treatment of neoplastic disease in the mammalian subject. Peptides, peptide analogs, or peptide-mimetics are provided which interact with tubulin to promote microtubule assembly, inhibit microtubule disassembly, or decrease thermal aggregation of tubulin, and therein can interrupt mitosis, preventing cell division, and triggering apoptosis in cells in a mammalian subject. Modulation of microtubule assembly is a cellular mechanism which can be regulated and lead to the development of new methods for treatment of cancer. The αB crystallin peptides LTITSSLSSDGV and ERTIPITRE disrupt tubulin [symbol] microtubule dynamics and have the potential to be developed into safe new therapeutics for cancer, Alzheimer's disease, and taupathies. In this study, the importance of five αB crystallin interactive sequences ₄₁STSLSPFYLRPPSFLRAP₅₈ (ST), ₇₃DRFSVNLDVKHFS₈₅ (DR), ₁₁₃FISREFHR₁₂₀ (FI), ₁₃₁LTITSSLSSDGV₁₄₂ (LT), and ₁₅₆ERTIPITRE₁₆₄ (ER) in the assembly/disassembly of microtubules and the thermal aggregation of tubulin was evaluated using synthetic αB crystallin peptides and αB crystallin mutants. The αB crystallin interactive sequences ₁₃₁LTITSSLSSDGV₁₄₂ and ₁₅₆ERTIPITRE₁₆₄ interact with tubulin to promote microtubule assembly and inhibit microtubule disassembly, while the interaction of the ₁₁₃FISREFHR₁₂₀ sequence with tubulin inhibited both microtubule assembly and disassembly. The remaining two peptides, ₄₁STSLSPFYLRPPSFLRAP₅₈ and ₇₃DRFSVNLDVKHFS₈₅ had little or no effect on microtubule assembly or disassembly. Microtubule assembly varied with the ratio of tubulin to αB crystallin resolving the apparent contradictions in the results of an αB crystallin effect on tubulin assembly. Xi et al., Faseb J 20:846-57, 2006; Atomi et al., Biol Sci Space 15:206-7, 2001; Mitra et al., Proteins, 2007. The observed effects of the αB crystallin synthetic peptides and the mutant αB crystallins are explained by the surface localization of the interactive sequences and the dynamic subunit model for chaperone activity. The collective response of interactive domains on the surface of αB crystallin appears to modulate the tubulin-microtubule dynamic equilibrium in a concentration dependent manner. Liu et al., Anal Biochem 350:186-95, 2006.

Polypeptides, peptide analogs and peptide mimetics of the non-native states of proteins are provided which interact with tubulin to promote microtubule assembly, inhibit microtubule disassembly, or decrease thermal aggregation of tubulin and induce apoptosis in cells in a mammalian subject. Accordingly, the present invention provides peptide-based compositions that promote microtubule assembly and are, therefore, useful in a variety of therapeutic applications, including, e.g., the treatment of diseases and disorders associated with microtubule assembly and the ability to induce apoptosis in cells in the mammalian subject, e.g., providing a treatment for neoplastic disease.

Novel functions for five interactive sequences in the small heat shock protein and molecular chaperone, human αB crystallin, were investigated in the assembly/disassembly of microtubules and aggregation of tubulin using synthetic peptides and mutant αB crystallins. The interactive sequence, ₁₁₃FISREFHR₁₂₀, exposed on the surface of αB crystallin decreased microtubule assembly by approximately 45%. In contrast, the interactive sequences, ₁₃₁LTITSSLSSDGV₁₄₂ and ₁₅₆ERTIPITRE₁₆₄, which correspond to the β8 strand and the C-terminus extension respectively and are involved in complex formation increased microtubule assembly by ˜34-45%. The αB crystallin peptides, ₁₁₃FISREFHR₁₂₀ and ₁₅₆ERTIPITRE₁₆₄ inhibited microtubule disassembly by ˜26-36%, and the peptides ₁₁₃FISREFHR₁₂₀ and ₁₃₁LTITSSLSSDGV₁₄₂ decreased the thermal aggregation of tubulin by ˜42-44%. The effects of the αB crystallin peptides on microtubule assembly/disassembly and tubulin aggregation were confirmed by mutagenesis of these interactive sequences in full-length human αB crystalline Microtubule assembly was dependent on the relative concentration of tubulin to αB crystalline. At molar ratios between 4:1 and 1:2 of tubulin to αB crystallin, microtubule assembly was promoted, while molar ratios <1:2 inhibited microtubule assembly. The dynamic subunit model for small heat shock protein function accounts for the modulation of microtubule assembly by αB crystalline.

Therapeutic applications for polypeptides, peptide analogs and peptide mimetics that interact with tubulin to promote microtubule assembly, inhibit microtubule disassembly, or decrease thermal aggregation of tubulin, and therein induce apoptosis in cells in a mammalian subject include, but are not limited to, treatment of neoplastic diseases, cancer, and solid tumors.

Protein pin arrays identified interactive polypeptide sequences for chaperone activity in human αB crystallin using natural lens proteins, β_{H} crystallin and γD crystallin, and in vitro chaperone target proteins, for example, alcohol dehydrogenase and citrate synthase. A polypeptide fragment having activity to interact with tubulin to promote microtubule assembly comprises polypeptide sequences from the α crystallin core domain or the C-terminal domain of the human αB crystallin protein.

The α crystallin core domain contained interactive protein sequences with tubulin assembly activity and chaperone activity, ₁₁₃FISREFHR₁₂₀, ₁₃₁LTITSSLS₁₃₈ (β8). The α crystallin core domain also contained interactive protein sequences with tubulin assembly activity and chaperone activity, for example, HGFISREF, EFHRKYRI, SREFHRKY. The C-terminal domain contained an interactive sequence, ₁₅₇RTIPITREL₁₆₄ that included the highly conserved I-X-I/V amino acid motif. The interactive sequence, ₁₃₁LTITSSLSDGV₁₄₁ belonging to the α crystallin core domain and ₁₅₇RTIPITRE₁₆₄ from the C-terminal domain were synthesized as peptides and assayed for tubulin assembly activity and chaperone activity in vitro. Both synthesized peptides inhibited the thermal aggregation of β_{H} crystallin, alcohol dehydrogenase and citrate synthase in vitro, The sequences, ₁₃₁LTITSSLSDGV₁₄₁ and ₁₅₇RTIPITRE₁₆₄ interacted with tubulin to promote microtubule assembly, inhibit microtubule disassembly, and/or decrease thermal aggregation of tubulin. The results suggested that interactive sequences in human αB crystallin have dual roles in tubulin interactive sequences with microtubule assembly/disassembly activity and chaperone activity.

It is to be understood that this invention is not limited to particular methods, reagents, compounds, compositions or biological systems, which can, of course, vary. It is also to be understood that the terminology used herein is for the purpose of describing particular embodiments only, and is not intended to be limiting. As used in this specification and the appended claims, the singular forms “a”, “an” and “the” include plural referents unless the content clearly dictates otherwise. Thus, for example, reference to “a cell” includes a combination of two or more cells, and the like.

“About” as used herein when referring to a measurable value such as an amount, a temporal duration, and the like, is meant to encompass variations of ±20% or +10%, more preferably ±5%, even more preferably ±1%, and still more preferably ±0.1% from the specified value, as such variations are appropriate to perform the disclosed methods.

Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, the preferred materials and methods are described herein. In describing and claiming the present invention, the following terminology will be used.

### Peptides

The peptides, peptide analogs and peptide-mimetics of the present invention, herein are collectively referred to as “Intellipeptides”, “peptides that interact with tubulin to increase microtubule assembly,” “peptides that interact with tubulin to inhibit microtubule disassembly”, or “peptides that interact with tubulin to decrease thermal aggregation of tubulin.” Intellipeptides are further referred to as “peptides that promote or inhibit microtubule assembly”, “peptides that inhibit abnormal protein folding, protein unfolding, protein misfolding, or protein aggregation.” Intellipeptides are identified using protein pin arrays, computer modeling, multiple sequence alignment analyses of structurally and functionally similar proteins, spectroscopic in vitro chaperone assays and/or in vivo cell killing assays.

Intellipeptides interact with tubulin to increase microtubule assembly or inhibit microtubule disassembly. Intellipeptides can also interact with tubulin to decrease thermal aggregation of tubulin.

In a method for treating a disease in a mammalian subject, Intellipeptides are useful to induce apoptosis in a cell in a mammalian subject by interaction of Intellipeptides with tubulin to increase microtubule assembly or inhibit microtubule disassembly or to decrease thermal aggregation of tubulin in a wide variety of disease target proteins. Disease targeting proteins include, but not limited to, neoplastic disease, cancer, and solid tumors.

“Neoplastic disease”, “cancer”, “malignancy”, “solid tumor” or “hyperproliferative disorder” are used as synonymous terms and refer to any of a number of diseases that are characterized by uncontrolled, abnormal proliferation of cells, the ability of affected cells to spread locally or through the bloodstream and lymphatic system to other parts of the body (i.e., metastasize) as well as any of a number of characteristic structural and/or molecular features. A “cancerous” or “malignant cell” or “solid tumor cell” is understood as a cell having specific structural properties, lacking differentiation and being capable of invasion and metastasis. “Neoplastic disease” or “cancer” refers to all types of cancer or neoplasm or malignant tumors found in mammals, including carcinomas, sarcomas, lymphomas and leukemias. Examples are cancers of the breast, lung, stomach, and oesophagus, brain and nervous system, head and neck, bone, liver, gall bladder, pancreas, colon, genitourinary system, urinary bladder, urinary system, kidney, testes, uterus, ovary, prostate, skin and skin appendices, melanoma, mesothelioma, endocrine system. (see DeVita, et al., (eds.), 2001, Cancer Principles and Practice of Oncology, 6th. Ed., Lippincott Williams & Wilkins, Philadelphia, Pa.; this reference is herein incorporated by reference in its entirety for all purposes).

Intellipeptides are provided which can comprise or consist of a fragment of αB crystalline In another embodiment, Intellipeptides of the present invention comprise or consist of peptides that are structurally and functionally similar to the parent set of peptide sequences identified from αB crystallin, including, but not limited to the peptides ₁₅₇RTIPITRE₁₆₄ or ₁₃₁LTITSSLSDGV₁₄₁. or functional variants or mimetics thereof. The present invention demonstrates that the parent set and peptide analogs and peptide mimetics of the parent set of these sequences interfere with or enhance the interaction with tubulin in assembly of microtubules, thus inhibiting or activating apoptosis in the cell. In addition, Intellipeptides stabilize microtubules or decrease thermal aggregation of tubulin.

Intellipeptides can include peptide analogs and peptide mimetics. Indeed, Intellipeptides include peptides having any of a variety of different modifications, including those described herein.

Intellipeptide analogs are generally designed and produced by chemical modifications of a lead peptide, including, e.g., any of the particular peptides described herein, such as any of the following sequences: i) DPLTITSSLSSDGVLTVNGPRKQ; ii) LTITSSLSDGVLTVNGPRK; iii) LTITSSLSDGV; iv) GPERTIPITREEK; v) PERTIPITREEK; vi) ERTIPITRE; or functional variants or peptide mimetics thereof. An exemplary polypeptide fragment of αB crystallin protein having tubulin binding and microtubule assembly activity and molecular chaperone activity is presented; e.g., the α crystallin core domain polypeptide fragment is ₁₁₃FISREFHR₁₂₀, ₁₃₁LTITSSLSSDGV₁₄₂ (β8), or the C-terminal domain polypeptide fragment is ₁₅₆ERTIPITRE₁₆₄, or functional variants or mimetics thereof. The present invention clearly establishes that these peptides in their entirety and derivatives created by modifying any side chains of the constituent amino acids have the ability to interact with tubulin to promote microtubule assembly, inhibit microtubule disassembly, and/or decrease thermal aggregation of tubulin. The present invention further encompasses polypeptides up to about 50 amino acids in length that include the amino acid sequences and functional variants or peptide mimetics of the sequences described herein.

Intellipeptides are provided which can include an N- and C-terminal modification. N-terminal acetylation or desamination confers protection against digestion by a number of aminopeptidases in the presence of amides or alcohols replacing the C-terminal carboxyl group prevent splitting by several carboxypeptidases, including carboxypeptidases A and B.

Intellipeptides are provided which can include a side-chain modification. The presence of non-natural amino acids usually increases peptide stability. In addition, at least one of these amino acids (α-aminoisobutyric acid or Aib) imposes significant constraints to model peptides diminishing their conformational flexibility. Therefore, the introduction of Aib is expected to enhance peptide stability and inhibitory activity at the same time.

Intellipeptides are provided which can include modifications in the α-carbon. The most commonly used alpha-carbon modification to improve peptide stability is α-methylation. In addition, replacement of the hydrogen atom linked to the α-carbon of Phe, Val or Leu favors the adoption of β-bend conformation that is unfavorable for the formation of β-pleated sheet structures. According to the present invention, methylation of those residues in the inhibitor peptides is expected to enhance stability and potency.

Intellipeptides are provided which can include a chirality change. Replacement of the natural L-residue by the D-enantiomers dramatically increases resistance to proteolytic degradation. Microtubule assembly promoters/inhibitors containing D-enantiomers are as effective in promoting or preventing microtubule assembly as the L-enantiomer forms of the promoter/inhibitor parent peptides.

Intellipeptides can be cyclic peptides. Conformationally constrained cyclic peptides represent better drug candidates than linear peptides due to their reduced conformational flexibility and improved resistance to exopeptidase cleavage. Two alternative strategies can be used to convert a linear sequence into a cyclic structure. One is the introduction of cysteine residue to achieve cyclization through the formation of a disulfide bridge and the other is the side-chain attachment strategy involving resin-bound head-to-tail cyclization. To avoid modifications of the peptide sequence the latter approach is used. Microtubule assembly promoter/inhibitor peptides contain the ideal sequences for facilitating macrocyclization because proline, due to its ability to promote turns and loops, is a constituent of many naturally occurring or artificially synthesized cyclic peptides.

Intellipeptides are provided which can be pseudopeptides. Pseudopeptides or amide bond surrogates refers to peptides containing chemical modifications of some (or all) of the peptide bonds. The introduction of amide bond surrogates not only decreases peptide degradation but also may significantly modify some of the biochemical properties of the peptides, particularly the conformational flexibility and hydrophobicity. It is likely that an increase in conformational flexibility will be beneficial for docking the inhibitor to the binding sites. On the other hand, since the interaction between the tubulin or microtubule proteins and the inhibitors seems to depend to a great extent on hydrophobic interactions, it is likely that amide bond replacement increasing hydrophobicity may enhance affinity and hence, potency of the inhibitors. In addition, increased hydrophobicity could also enhance transport of the peptide across membranes and thus, improve barrier permeability (blood-brain barrier and intestinal barrier). The amide bonds to replace are those located at the end of the peptide to prevent exoprotease degradation and after each of the prolines, since it has been described that a frequent endopeptidase cleavage site occurs after this residue by an enzyme known as prolylendopeptidase.

To improve or alter the characteristics of polypeptides of the present invention, protein engineering can be employed. Recombinant DNA technology known to those skilled in the art can be used to create novel mutant proteins or muteins including single or multiple amino acid substitutions, deletions, additions, or fusion proteins. Such modified polypeptides can show, e.g., increased/decreased biological activity or increased/decreased stability. In addition, they can be purified in higher yields and show better solubility than the corresponding natural polypeptide, at least under certain purification and storage conditions. Further, the polypeptides of the present invention can be produced as multimers including dimers, trimers and tetramers. Multimerization can be facilitated by linkers or recombinantly though heterologous polypeptides such as Fc regions.

It is known in the art that one or more amino acids can be deleted from the N-terminus or C-terminus without substantial loss of biological function. See, e.g., Ron, et al, Biol. Chem., 268: 2984-2988, 1993. Accordingly, the present invention provides polypeptides having one or more residues deleted from the amino terminus. Similarly, many examples of biologically functional C-terminal deletion mutants are known (see, e.g., Dobeli, et al., 1988). Accordingly, the present invention provides polypeptides having one or more residues deleted from the carboxy terminus. The invention also provides polypeptides having one or more amino acids deleted from both the amino and the carboxyl termini as described below.

Other mutants in addition to N- and C-terminal deletion forms of the protein discussed above are included in the present invention. Thus, the invention further includes variations of the polypeptides which show substantial chaperone polypeptide activity. Such mutants include deletions, insertions, inversions, repeats, and substitutions selected according to general rules known in the art so as to have little effect on activity.

There are two main approaches for studying the tolerance of an amino acid sequence to change, see, Bowie, et al., Science, 247: 1306-1310, 1994. The first method relies on the process of evolution, in which mutations are either accepted or rejected by natural selection. The second approach uses genetic engineering to introduce amino acid changes at specific positions of a cloned gene and selections or screens to identify sequences that maintain functionality. These studies have revealed that proteins are surprisingly tolerant of amino acid substitutions.

Typically seen as conservative substitutions are the replacements, one for another, among the aliphatic amino acids Ala, Val, Leu and Phe; interchange of the hydroxyl residues Ser and Thr, exchange of the acidic residues Asp and Glu, substitution between the amide residues Asn and Gln, exchange of the basic residues Lys and Arg and replacements among the aromatic residues Phe, Tyr. Thus, the polypeptide of the present invention can be, for example: (i) one in which one or more of the amino acid residues are substituted with a conserved or non-conserved amino acid residue (preferably a conserved amino acid residue) and such substituted amino acid residue can or cannot be one encoded by the genetic code; or (ii) one in which one or more of the amino acid residues includes a substituent group; or (iii) one in which the PEDF-R polypeptide is fused with another compound, such as a compound to increase the half-life of the polypeptide (for example, polyethylene glycol); or (iv) one in which the additional amino acids are fused to the above form of the polypeptide, such as an IgG Fc fusion region peptide or leader or secretory sequence or a sequence which is employed for purification of the above form of the polypeptide or a pro-protein sequence.

Thus, the polypeptides of the present invention can include one or more amino acid substitutions, deletions, or additions, either from natural mutations or human manipulation. As indicated, changes are preferably of a minor nature, such as conservative amino acid substitutions that do not significantly affect the folding or activity of the protein. The following groups of amino acids represent equivalent changes: (1) Ala, Pro, Gly, Glu, Asp, Gln, Asn, Ser, Thr; (2) Cys, Ser, Tyr, Thr; (3) Val, Ile, Leu, Met, Ala, Phe; (4) Lys, Arg, His; (5) Phe, Tyr, Trp, His.

Furthermore, polypeptides of the present invention can include one or more amino acid substitutions that mimic modified amino acids. An example of this type of substitution includes replacing amino acids that are capable of being phosphorylated (e.g., serine, threonine, or tyrosine) with a negatively charged amino acid that resembles the negative charge of the phosphorylated amino acid (e.g., aspartic acid or glutamic acid). Also included is substitution of amino acids that are capable of being modified by hydrophobic groups (e.g., arginine) with amino acids carrying bulky hydrophobic side chains, such as tryptophan or phenylalanine. Therefore, a specific embodiment of the invention includes chaperone polypeptides that include one or more amino acid substitutions that mimic modified amino acids at positions where amino acids that are capable of being modified are normally positioned. Further included are chaperone polypeptides where any subset of modifiable amino acids is substituted. For example, a chaperone polypeptide that includes three serine residues can be substituted at any one, any two, or all three of said serines. Furthermore, any chaperone polypeptide amino acid capable of being modified can be excluded from substitution with a modification-mimicking amino acid.

The present invention is further directed to fragments of the polypeptides of the present invention. More specifically, the present invention embodies purified, isolated, and recombinant polypeptides comprising at least any one integer between 6 and 504 (or the length of the polypeptides amino acid residues minus 1 if the length is less than 1000) of consecutive amino acid residues. Preferably, the fragments are at least 6, preferably at least 8 to 10, more preferably 12, 15, 20, 25, 30, 35, 40, 50, 60, 75, 100, 125, 150, 175, 200, 225, 250, 275, 300, 325, 350, 360, or more consecutive amino acids of a polypeptide of the present invention.

The present invention also provides for the exclusion of any species of polypeptide fragments of the present invention specified by 5′ and 3′ positions or sub-genuses of polypeptides specified by size in amino acids as described above. Any number of fragments specified by 5′ and 3′ positions or by size in amino acids, as described above, can be excluded.

In addition, it should be understood that in certain embodiments, Intellipeptides of the present invention include two or more modifications, including, but not limited to those described herein. By taking into the account the features of the peptide drugs on the market or under current development, it is clear that most of the peptides successfully stabilized against proteolysis consist of a mixture of several types of the above described modifications. This conclusion is understood in the light of the knowledge that many different enzymes are implicated in peptide degradation.

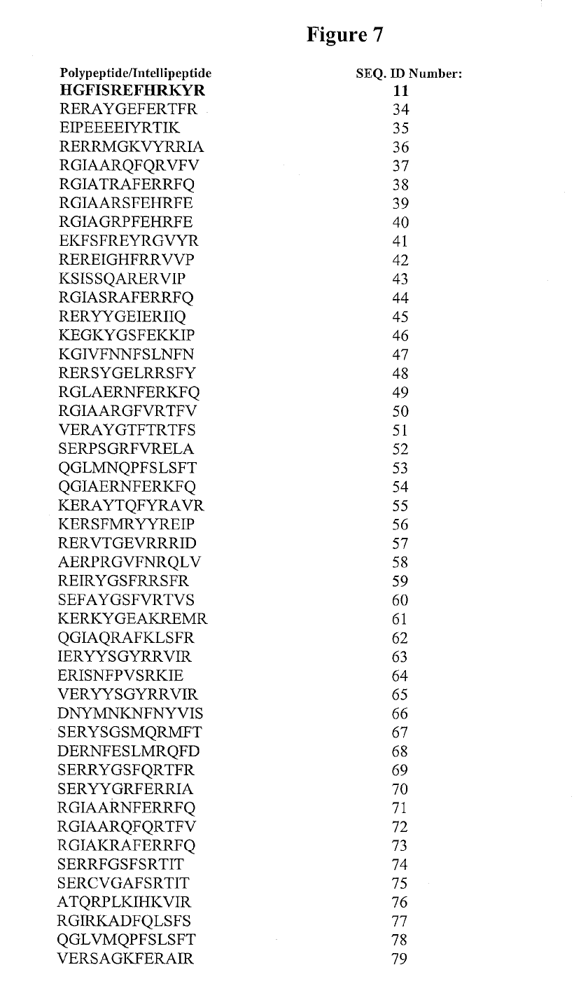

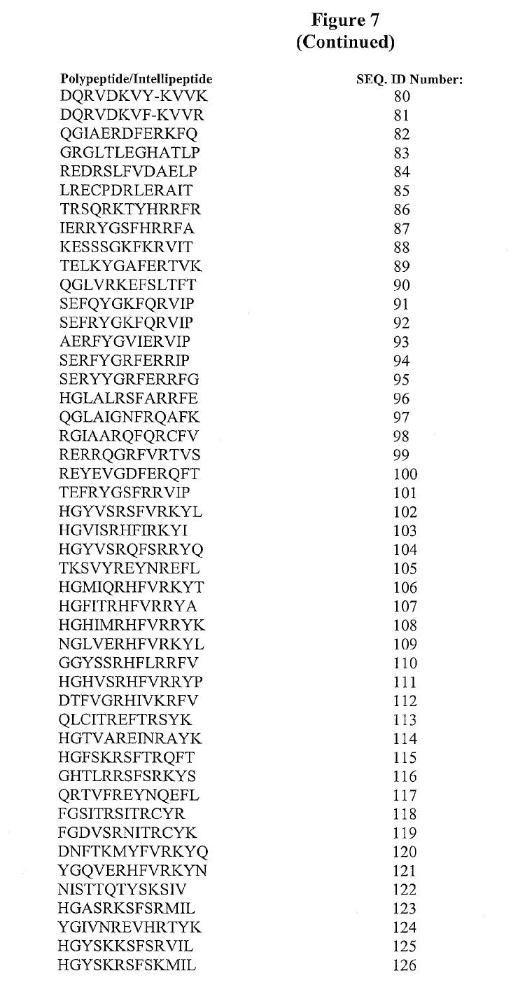

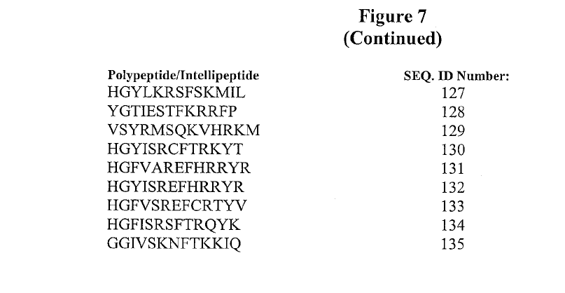

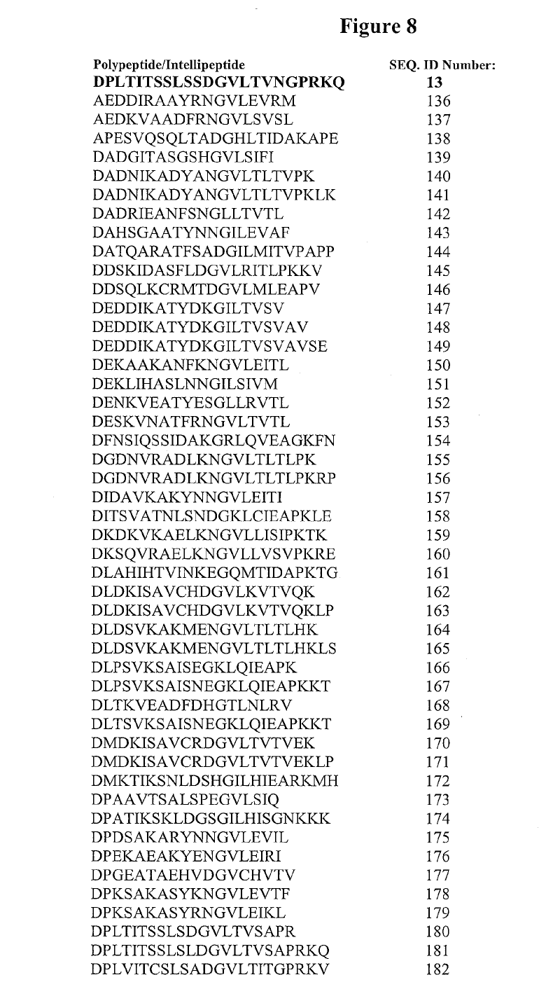

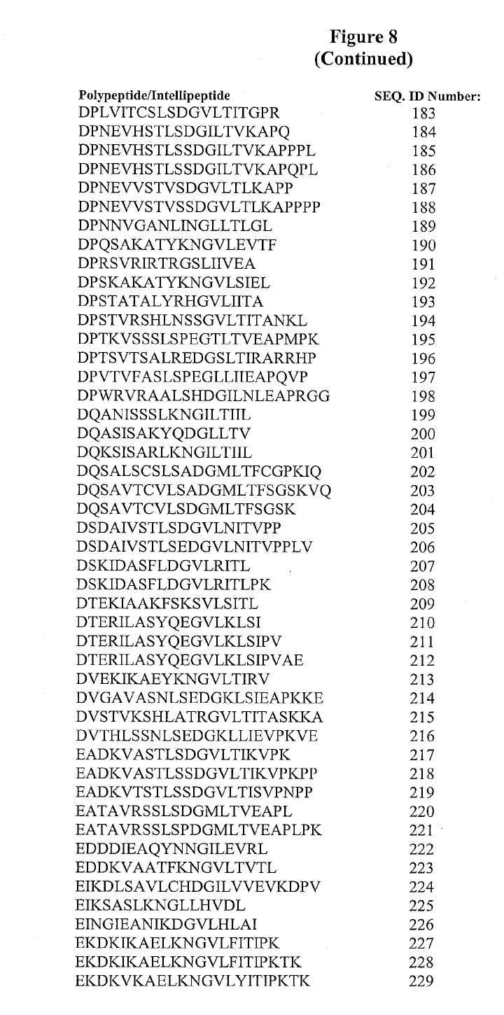

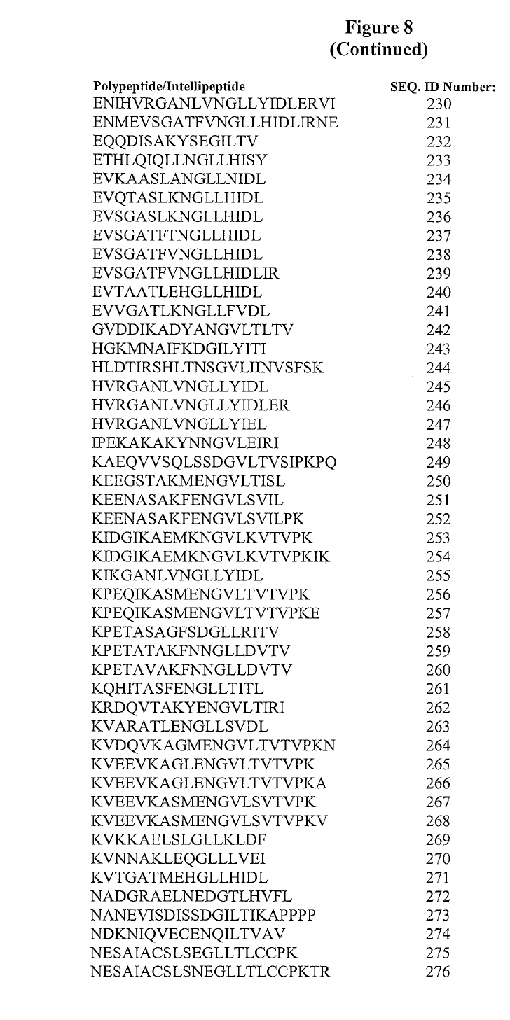

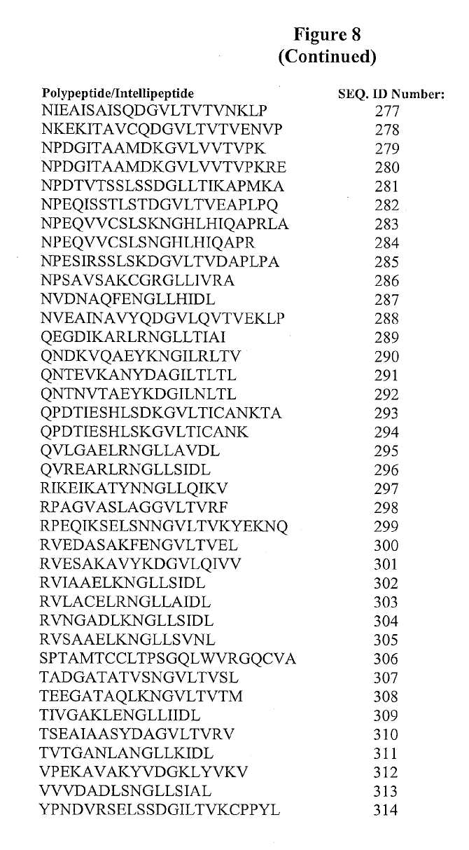

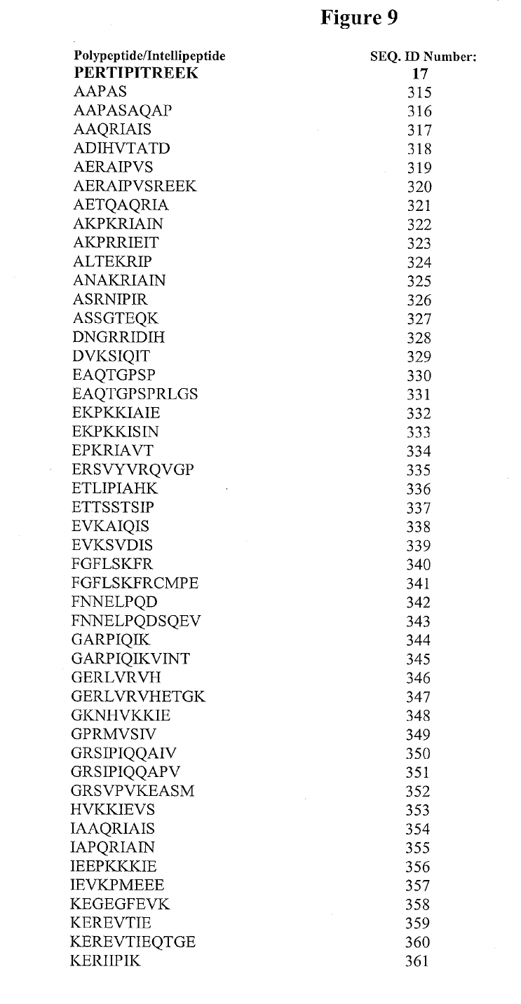

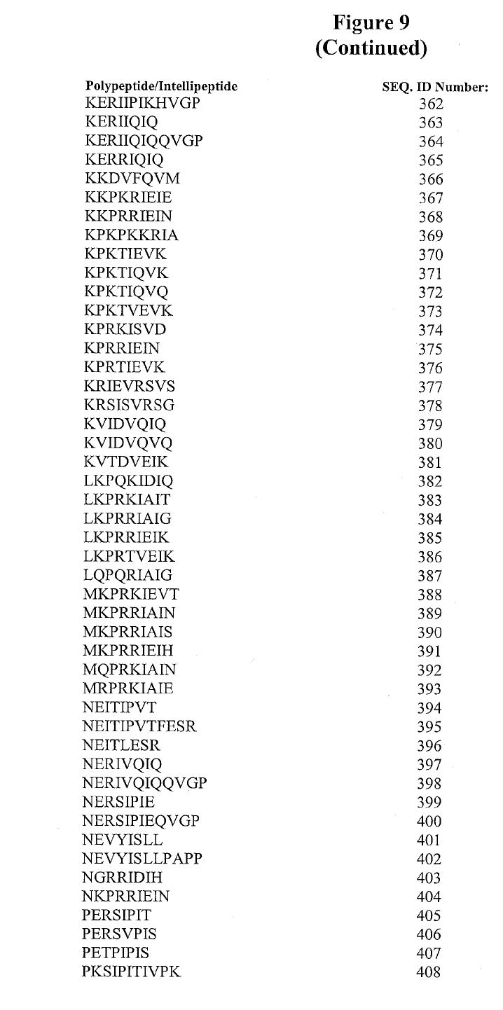

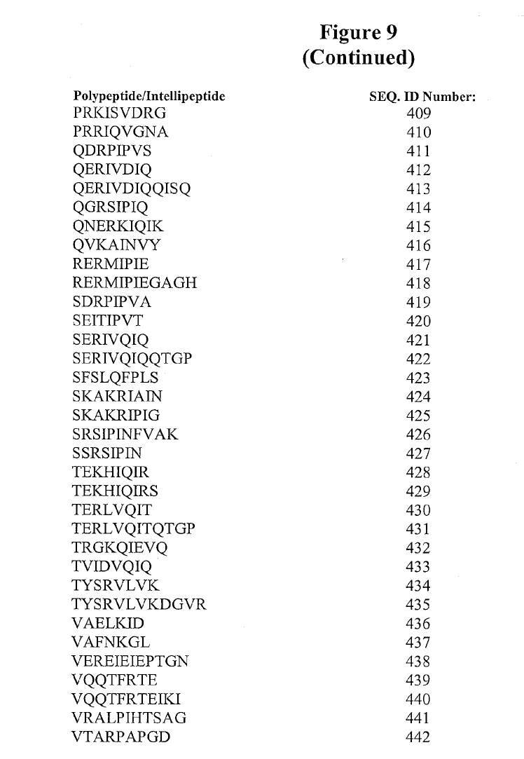

Libraries of Intellipeptides, or polypeptides, peptide analogs, or peptide-mimetics are provided herein which interact with tubulin to promote microtubule assembly, inhibit microtubule disassembly, or decrease thermal aggregation of tubulin, and therein induce apoptosis in cells in a mammalian subject. Such libraries include both peptide libraries and libraries of nucleic acid constructs capable of expressing Intellipeptides. In one embodiment, a library of the present invention consists of sequences related to i) DPLTITSSLSSDGVLTVNGPRKQ; ii) LTITSSLSDGVLTVNGPRK; iii) LTITSSLSDGV; iv) GPERTIPITREEK; v) PERTIPITREEK; vi) ERTIPITRE; or functional variants or peptide mimetics thereof. In a particular embodiment, a library of the invention consists of two or more Intellipeptides or encoding sequences, including, e.g., the sequences provided in FIG. 7 (LTITSSLSDGV), FIG. 8 (ERTIPITRE), or FIG. 9. (FISREFHR).

### Cancer Treatment

Neoplastic disease”, “cancer”, “malignancy”, “solid tumor” or “hyperproliferative disorder” are used as synonymous terms and refer to any of a number of diseases that are characterized by uncontrolled, abnormal proliferation of cells, the ability of affected cells to spread locally or through the bloodstream and lymphatic system to other parts of the body (i.e., metastasize) as well as any of a number of characteristic structural and/or molecular features. “Metastatic” refers to tumor cells as defined above which spread to other organs or to distant sites of the same organ.

“Cancer-associated” refers to the relationship of a nucleic acid and its expression, or lack thereof, or a protein and its level or activity, or lack thereof, to the onset of malignancy in a subject cell. For example, cancer can be associated with expression of a particular gene that is not expressed, or is expressed at a lower level, in a normal healthy cell. Conversely, a cancer-associated gene can be one that is not expressed in a malignant cell (or in a cell undergoing transformation), or is expressed at a lower level in the malignant cell than it is expressed in a normal healthy cell.

In the context of the cancer, the term “transformation” refers to the change that a normal cell undergoes as it becomes malignant. In eukaryotes, the term “transformation” can be used to describe the conversion of normal cells to malignant cells in cell culture.

“Proliferating cells” are those which are actively undergoing cell division and growing exponentially. “Loss of cell proliferation control” refers to the property of cells that have lost the cell cycle controls that normally ensure appropriate restriction of cell division. Cells that have lost such controls proliferate at a faster than normal rate, without stimulatory signals, and do not respond to inhibitory signals.

“Advanced cancer” means cancer that is no longer localized to the primary tumor site, or a cancer that is Stage III or IV according to the American Joint Committee on Cancer (AJCC).

“Well tolerated” refers to the absence of adverse changes in health status that occur as a result of the treatment and would affect treatment decisions.

“Metastatic” refers to tumor cells, e.g., human solid tumor or genitourinary malignancy, that are able to establish secondary tumor lesions in the lungs, liver, bone or brain of immune deficient mice upon injection into the mammary fat pad and/or the circulation of the immune deficient mouse.

Intellepeptides, e.g., ₁₁₃FISREFHR₁₂₀, ₁₃₁LTITSSLSSDGV₁₄₂ (β8), or ₁₅₆ERTIPITRE₁₆₄, or functional variants or mimetics thereof are useful in a method of treating disease, for example, neoplastic disease. A “solid tumor” includes, but is not limited to, sarcoma, melanoma, carcinoma, or other solid tumor cancer.

“Sarcoma” refers to a tumor which is made up of a substance like the embryonic connective tissue and is generally composed of closely packed cells embedded in a fibrillar or homogeneous substance. Sarcomas include, but are not limited to, chondrosarcoma, fibrosarcoma, lymphosarcoma, melanosarcoma, myxosarcoma, osteosarcoma, Abemethy's sarcoma, adipose sarcoma, liposarcoma, alveolar soft part sarcoma, ameloblastic sarcoma, botryoid sarcoma, chloroma sarcoma, chorio carcinoma, embryonal sarcoma, Wilms' tumor sarcoma, endometrial sarcoma, stromal sarcoma, Ewing's sarcoma, fascial sarcoma, fibroblastic sarcoma, giant cell sarcoma, granulocytic sarcoma, Hodgkin's sarcoma, idiopathic multiple pigmented hemorrhagic sarcoma, immunoblastic sarcoma of B cells, lymphoma, immunoblastic sarcoma of T-cells, Jensen's sarcoma, Kaposi's sarcoma, Kupffer cell sarcoma, angiosarcoma, leukosarcoma, malignant mesenchymoma sarcoma, parosteal sarcoma, reticulocytic sarcoma, Rous sarcoma, serocystic sarcoma, synovial sarcoma, and telangiectatic sarcoma.

“Melanoma” refers to a tumor arising from the melanocytic system of the skin and other organs. Melanomas include, for example, acral-lentiginous melanoma, amelanotic melanoma, benign juvenile melanoma, Cloudman's melanoma, S91 melanoma, Harding-Passey melanoma, juvenile melanoma, lentigo maligna melanoma, malignant melanoma, nodular melanoma, subungal melanoma, and superficial spreading melanoma.

“Carcinoma” refers to a malignant new growth made up of epithelial cells tending to infiltrate the surrounding tissues and give rise to metastases. Exemplary carcinomas include, for example, acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basaloid carcinoma, basosquamous cell carcinoma, bronchioalveolar carcinoma, bronchiolar carcinoma, bronchogenic carcinoma, cerebriform carcinoma, cholangiocellular carcinoma, chorionic carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epiermoid carcinoma, carcinoma epitheliale adenoides, exophytic carcinoma, carcinoma ex ulcere, carcinoma fibrosum, gelatiniform carcinoma, gelatinous carcinoma, giant cell carcinoma, carcinoma gigantocellulare, glandular carcinoma, granulosa cell carcinoma, hair-matrix carcinoma, hematoid carcinoma, hepatocellular carcinoma, Hurthle cell carcinoma, hyaline carcinoma, hypemephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidernoid carcinoma, carcinoma mucosum, mucous carcinoma, carcinoma myxomatodes, naspharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, schneiderian carcinoma, scirrhous carcinoma, carcinoma scroti, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tuberous carcinoma, verrucous carcinoma, and carcinoma viflosum.

“Leukemia” refers to progressive, malignant diseases of the blood-forming organs and is generally characterized by a distorted proliferation and development of leukocytes and their precursors in the blood and bone marrow. Leukemia is generally clinically classified on the basis of (1) the duration and character of the disease—acute or chronic; (2) the type of cell involved; myeloid (myelogenous), lymphoid (lymphogenous), or monocytic; and (3) the increase or non-increase in the number of abnormal cells in the blood—leukemic or aleukemic (subleukemic). Leukemia includes, for example, acute nonlymphocytic leukemia, chronic lymphocytic leukemia, acute granulocytic leukemia, chronic granulocytic leukemia, acute promyelocytic leukemia, adult T-cell leukemia, aleukemic leukemia, a leukocythemic leukemia, basophylic leukemia, blast cell leukemia, bovine leukemia, chronic myelocytic leukemia, leukemia cutis, embryonal leukemia, eosinophilic leukemia, Gross' leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocytic leukemia, micromyeloblastic leukemia, monocytic leukemia, myeloblastic leukemia, myelocytic leukemia, myeloid granulocytic leukemia, myelomonocytic leukemia, Naegeli leukemia, plasma cell leukemia, plasmacytic leukemia, promyelocytic leukemia, Rieder cell leukemia, Schilling's leukemia, stem cell leukemia, subleukemic leukemia, and undifferentiated cell leukemia.

Additional cancers include, for example, Hodgkin's Disease, Non-Hodgkin's Lymphoma, multiple myeloma, neuroblastoma, breast cancer, ovarian cancer, lung cancer, rhabdomyosarcoma, primary thrombocytosis, primary macroglobulinemia, small-cell lung tumors, primary brain tumors, stomach cancer, colon cancer, malignant pancreatic insulanoma, malignant carcinoid, urinary bladder cancer, premalignant skin lesions, testicular cancer, lymphomas, thyroid cancer, neuroblastoma, esophageal cancer, genitourinary tract cancer, malignant hypercalcemia, cervical cancer, endometrial cancer, adrenal cortical cancer, and prostate cancer.

“Treating” refers to any indicia of success in the treatment or amelioration or prevention of an cancer, including any objective or subjective parameter such as abatement; remission; diminishing of symptoms or making the disease condition more tolerable to the patient; slowing in the rate of degeneration or decline; or making the final point of degeneration less debilitating. The treatment or amelioration of symptoms can be based on objective or subjective parameters; including the results of an examination by a physician. Accordingly, the term “treating” includes the administration of the compounds or agents of the present invention to prevent or delay, to alleviate, or to arrest or inhibit development of the symptoms or conditions associated with disease, e.g., neoplastic disease. The term “therapeutic effect” refers to the reduction, elimination, or prevention of the disease, symptoms of the disease, or side effects of the disease in the subject.

“In combination with”, “combination therapy” and “combination products” refer, in certain embodiments, to the concurrent administration to a patient of a first therapeutic and the compounds as used herein. When administered in combination, each component can be administered at the same time or sequentially in any order at different points in time. Thus, each component can be administered separately but sufficiently closely in time so as to provide the desired therapeutic effect. “Concomitant administration” of a known cancer therapeutic drug with a pharmaceutical composition of the present invention means administration of the drug and the peptide, peptide analog or peptide mimetic composition at such time that both the known drug and the composition of the present invention will have a therapeutic effect. Such concomitant administration can involve concurrent (i.e. at the same time), prior, or subsequent administration of the cancer therapeutic drug with respect to the administration of a compound of the present invention. A person of ordinary skill in the art, would have no difficulty determining the appropriate timing, sequence and dosages of administration for particular drugs and compositions of the present invention.

“Dosage unit” refers to physically discrete units suited as unitary dosages for the particular individual to be treated. Each unit can contain a predetermined quantity of active compound(s) calculated to produce the desired therapeutic effect(s) in association with the required pharmaceutical carrier. The specification for the dosage unit forms can be dictated by (a) the unique characteristics of the active compound(s) and the particular therapeutic effect(s) to be achieved, and (b) the limitations inherent in the art of compounding such active compound(s).

### Neurodegenerative Diseases

Many neurodegenerative diseases run their course with the presence of abnormal intra- and extracellular protein aggregates and can benefit from treatment with peptides that interact with tubulin to promote microtubule assembly, inhibit microtubule disassembly, or decrease thermal aggregation of tubulin. Such diseases include, but are not limited to, taupathies, Alzheimer's disease, motor neuron disease, hypoparathyroidism-retardation-dysmorphic syndrome, Parkinson's disease, schizophrenia, or Lewy body diseases. Among the proteins that are frequently found to be aggregated in such pathologies, there are two that are very interesting: α-synuclein and tau protein, which give rise to α-synucleinopathies and taupathies, respectively. The α-synucleinopathies include Parkinson's disease, dementia with Lewy bodies, and multiple system atrophy. The most extensively studied taupathies include progressive supranuclear palsy, Pick's disease, corticobasal degeneration and frontotemporal dementias. Alzheimer's disease shows features common to both groups of pathologies. In many of these pathologies, alterations of neurotransmitters and their cell signalling pathways have been reported, mainly including the cholinergic pathways, although little is known with respect to the glutamatergic and adenosinergic pathways. Glutamate, which is involved in learning and memory processes, is the main excitatory neurotransmitter of the central nervous system, acting through ionotropic and metabotropic receptors. However, glutamate acts at high concentrations as a neurotoxin, causing degeneration and cell death. The release of this neurotransmitter is regulated by the adenosine nucleoside which, acting through type A1 receptors, produces an inhibition of glutamate release and thus plays a neuroprotective role. The ionotropic receptors are involved in the excitotoxicity of glutamate; however, a neuroprotective role has been attributed to the metabotropic receptors. The aim of this project is to study the modulation of the metabotropic receptors of glutamate and adenosine A1 in the post mortem human brain of individuals with various neurodegenerative pathologies, including Alzheimer's or Parkinson's disease, dementia with Lewy bodies (common and pure forms), progressive supranuclear palsy, Pick's disease, argyrophilic grain disease, corticobasal degeneration, etc., comparing it to the brain of healthy individuals used as controls. The study will be carried out on all the various components of the glutamatergic and adenosinergic pathways, including the receptors themselves, G proteins, adenylate cyclase and phospholipase C, and a study will be made of various molecular aggregates including the proteins involved in the cell signalling mediated by these receptors and abnormal proteins present in these pathologies, such as α-synuclein and tau deposits. The purpose of this is to verify the differences between these neurodegenerative pathologies in order to open new possibilities for the design of therapeutic targets.

### Peptides, Peptide Variants, and Peptide Mimetics

The invention provides isolated or recombinant polypeptides comprising an amino acid sequence having at least 95%, 96%, 97%, 98%, 99% or more sequence identity to a polypeptide fragment of an N-terminal domain, an α crystallin core domain, or a C-terminal domain of the αB crystallin protein over a region of at least about 10, 50, 100, 150, or 200, or more residues, or, a polypeptide encoded by a nucleic acid of the invention. In one aspect, the polypeptide comprises a sequence as set forth in a polypeptide fragment of an N-terminal domain, an α crystallin core domain, or a C-terminal domain of the αB crystallin protein. The invention provides methods for treating neoplastic disease in a mammalian subject by administering a polypeptide fragment of αB crystallin protein, e.g., a polypeptide of the invention. The invention also provides methods for screening for compositions that have chaperone activity or that interact with tubulin to promote microtubule assembly, inhibit microtubule disassembly, or decrease thermal aggregation of tubulin, and therein induce apoptosis in cells in a mammalian subject by screening polypeptide fragments of αB crystallin protein, e.g., a polypeptide of the invention.

In one aspect, the invention provides a polypeptide fragment of αB crystallin protein (and the nucleic acids encoding them) where one, some or all of the amino acids in the polypeptide fragment of αB crystallin protein comprise replacements with substituted amino acids. In one aspect, the invention provides methods to enhance the interaction of a polypeptide fragment of αB crystallin protein having molecular chaperone activity with unfolded proteins, denatured proteins, or native conformation proteins.

The peptides and polypeptides of the invention can be expressed recombinantly in vivo after administration of nucleic acids, as described above, or, they can be administered directly, e.g., as a pharmaceutical composition. They can be expressed in vitro or in vivo to screen for polypeptide fragments of αB crystallin protein having molecular chaperone activity activity and for agents that can ameliorate disease, for example, the treatment of diseases and disorders associated with microtubule assembly and the ability to induce apoptosis in cells in the mammalian subject, e.g., treatment of neoplastic diseases, cancer, and solid tumors.

Polypeptides and peptides are provided which can be isolated from natural sources, be synthetic, or be recombinantly generated polypeptides. Peptides and proteins can be recombinantly expressed in vitro or in vivo. The peptides and polypeptides of the invention can be made and isolated using any method known in the art. Polypeptide and peptides of the invention can also be synthesized, whole or in part, using chemical methods well known in the art. See e.g., Caruthers, Nucleic Acids Res. Symp. Ser. 215-223, 1980; Horn, Nucleic Acids Res. Symp. Ser. 225-232, 1980; Banga, Therapeutic Peptides and Proteins, Formulation, Processing and Delivery Systems Technomic Publishing Co., Lancaster, Pa., 1995. For example, peptide synthesis can be performed using various solid-phase techniques (see e.g., Roberge, Science 269: 202, 1995; Merrifield, Methods Enzymol. 289: 3-13, 1997) and automated synthesis can be achieved, e.g., using the ABI 431A Peptide Synthesizer (Perkin Elmer) in accordance with the instructions provided by the manufacturer.

The peptides and polypeptides, as defined above, include all “mimetic” and “peptidomimetic” forms. The terms “mimetic” and “peptidomimetic” refer to a synthetic chemical compound which has substantially the same structural and/or functional characteristics of the polypeptides of the invention. The mimetic can be either entirely composed of synthetic, non-natural analogues of amino acids, or, is a chimeric molecule of partly natural peptide amino acids and partly non-natural analogs of amino acids. The mimetic can also incorporate any amount of natural amino acid conservative substitutions as long as such substitutions also do not substantially alter the mimetic's structure and/or activity. As with polypeptides of the invention which are conservative variants, routine experimentation will determine whether a mimetic is within the scope of the invention, i.e., that its structure and/or function is not substantially altered. Thus, a mimetic composition is within the scope of the invention if, when administered to or expressed in a cell, e.g., a polypeptide fragment of αB crystallin protein having molecular chaperone activity. A mimetic composition can also be within the scope of the invention if it stimulates a molecular chaperone activity in a cell or mammalian subject with diseases and disorders associated with microtubule assembly and the ability to induce apoptosis in cells in the mammalian subject, including, but not limited to, treatment of neoplastic diseases, cancer, and solid tumors.

A method is provided for designing a polypeptide mimetic using a molecular model of an electrostatic surface to design a synthetic molecule with characteristics of a polypeptide. Using molecular modeling one can construct an amino acid map of the peptide of interest. From the amino acid map, one can compute an electrostatic surface around the peptide. By removing the amino acid map from the electrostatic surface map, one can use the electrostatic surface to design a synthetic molecule with the same shape, size and charge characteristics as a polypeptide.

Intellipeptides or peptides that inhibit abnormal protein folding, protein unfolding, protein misfolding, or microtubule assembly promoter/inhibitor include, but are not limited to, Intellipeptides i) DPLTITSSLSSDGVLTVNGPRKQ; ii) LTITSSLSDGVLTVNGPRK; iii) LTITSSLSDGV; iv) GPERTIPITREEK; v) PERTIPITREEK; vi) ERTIPITRE; or functional variants or peptide mimetics thereof.

Polypeptide mimetic compositions can contain any combination of non-natural structural components, which are typically from three structural groups: a) residue linkage groups other than the natural amide bond (“peptide bond”) linkages; b) non-natural residues in place of naturally occurring amino acid residues; or c) residues which induce secondary structural mimicry, i.e., to induce or stabilize a secondary structure, e.g., a beta turn, gamma turn, beta sheet, alpha helix conformation, and the like. For example, a polypeptide can be characterized as a mimetic when all or some of its residues are joined by chemical means other than natural peptide bonds. Individual peptidomimetic residues can be joined by peptide bonds, other chemical bonds or coupling means, such as, e.g., glutaraldehyde, N-hydroxysuccinimide esters, bifunctional maleimides, N,N′-dicyclohexylcarbodiimide (DCC) or N,N′-diisopropylcarbodiimide (DIC). Linking groups that can be an alternative to the traditional amide bond (“peptide bond”) linkages include, e.g., ketomethylene (e.g., —C(═O)—CH₂— for —C(═O)—NH—), aminomethylene (CH₂—NH), ethylene, olefin (CH═CH), ether (CH₂—O), thioether (CH₂—S), tetrazole (CN₄—), thiazole, retroamide, thioamide, or ester (see, e.g., Spatola (1983) in Chemistry and Biochemistry of Amino Acids, Peptides and Proteins, Vol. 7, pp 267-357, “Peptide Backbone Modifications,” Marcell Dekker, NY).

A polypeptide can also be characterized as a mimetic by containing all or some non-natural residues in place of naturally occurring amino acid residues. Non-natural residues are well described in the scientific and patent literature; a few exemplary non-natural compositions useful as mimetics of natural amino acid residues and guidelines are described below. Mimetics of aromatic amino acids can be generated by replacing by, e.g., D- or L-naphylalanine; D- or L-phenylglycine; D- or L-2 thieneylalanine; D- or L-1, -2,3-, or 4-pyreneylalanine; D- or L-3 thieneylalanine; D- or L-(2-pyridinyl)-alanine; D- or L-(3-pyridinyl)-alanine; D- or L-(2-pyrazinyl)-alanine; D- or L-(4-isopropyl)-phenylglycine; D-(trifluoromethyl)-phenylglycine; D-(trifluoromethyl)-phenylalanine; D-p-fluoro-phenylalanine; D- or L-p-biphenylphenylalanine; K- or L-p-methoxy-biphenylphenylalanine; D- or L-2-indole(alkyl)alanines; and, D- or L-alkylainines, where alkyl can be substituted or unsubstituted methyl, ethyl, propyl, hexyl, butyl, pentyl, isopropyl, iso-butyl, sec-isotyl, iso-pentyl, or a non-acidic amino acids. Aromatic rings of a non-natural amino acid include, e.g., thiazolyl, thiophenyl, pyrazolyl, benzimidazolyl, naphthyl, furanyl, pyrrolyl, and pyridyl aromatic rings.

Mimetics of acidic amino acids can be generated by substitution by, e.g., non-carboxylate amino acids while maintaining a negative charge; (phosphono)alanine; sulfated threonine. Carboxyl side groups (e.g., aspartyl or glutamyl) can also be selectively modified by reaction with carbodiimides (R′—N—C—N—R′) such as, e.g., 1-cyclohexyl-3(2-morpholin-yl-(4-ethyl) carbodiimide or 1-ethyl-3(4-azonia-4,4-dimetholpentyl) carbodiimide. Aspartyl or glutamyl can also be converted to asparaginyl and glutaminyl residues by reaction with ammonium ions.

Mimetics of basic amino acids can be generated by substitution with, e.g., (in addition to lysine and arginine) the amino acids ornithine, citrulline, or (prolyl)-acetic acid, or (prolyl)alkyl-acetic acid, where alkyl is defined above. Nitrile derivative (e.g., containing the CN-moiety in place of COOH) can be substituted for proline or glutamine. Asparaginyl and glutaminyl residues can be deaminated to the corresponding aspartyl or glutamyl residues.

Arginine residue mimetics can be generated by reacting arginyl with, e.g., one or more conventional reagents, including, e.g., phenylglyoxal, 2,3-butanedione, 1,2-cyclohexanedione, or ninhydrin, preferably under alkaline conditions. Tyrosine residue mimetics can be generated by reacting tyrosyl with, e.g., aromatic diazonium compounds or tetranitromethane. N-acetylimidizol and tetranitromethane can be used to form O-acetyl tyrosyl species and 3-nitro derivatives, respectively. Cysteine residue mimetics can be generated by reacting cysteinyl residues with, e.g., alpha-haloacetates such as 2-chloroacetic acid or chloroacetamide and corresponding amines; to give carboxymethyl or carboxyamidomethyl derivatives. Cysteine residue mimetics can also be generated by reacting cysteinyl residues with, e.g., bromo-trifluoroacetone, alpha-bromo-beta-(5-imidozoyl) propionic acid; chloroacetyl phosphate, N-alkylmaleimides, 3-nitro-2-pyridyl disulfide; methyl 2-pyridyl disulfide; p-chloromercuribenzoate; 2-chloromercuri-4 nitrophenol; or, chloro-7-nitrobenzo-oxa-1,3-diazole. Lysine mimetics can be generated (and amino terminal residues can be altered) by reacting lysinyl with, e.g., succinic or other carboxylic acid anhydrides. Lysine and other alpha-amino-containing residue mimetics can also be generated by reaction with imidoesters, such as methyl picolinimidate, pyridoxal phosphate, pyridoxal, chloroborohydride, trinitrobenzenesulfonic acid, O-methylisourea, 2,4, pentanedione, and transamidase-catalyzed reactions with glyoxylate. Mimetics of methionine can be generated by reaction with, e.g., methionine sulfoxide. Mimetics of proline include, e.g., pipecolic acid, thiazolidine carboxylic acid, 3- or 4-hydroxy proline, dehydroproline, 3- or 4-methylproline, or 3,3,-dimethylproline. Histidine residue mimetics can be generated by reacting histidyl with, e.g., diethylprocarbonate or para-bromophenacyl bromide. Other mimetics include, e.g., those generated by hydroxylation of proline and lysine; phosphorylation of the hydroxyl groups of seryl or threonyl residues; methylation of the alpha-amino groups of lysine, arginine and histidine; acetylation of the N-terminal amine; methylation of main chain amide residues or substitution with N-methyl amino acids; or amidation of C-terminal carboxyl groups.

A component of a polypeptide of the invention can also be replaced by an amino acid (or peptidomimetic residue) of the opposite chirality. Thus, any amino acid naturally occurring in the L-configuration (which can also be referred to as the R or S, depending upon the structure of the chemical entity) can be replaced with the amino acid of the same chemical structural type or a peptidomimetic, but of the opposite chirality, referred to as the D-amino acid, but which can additionally be referred to as the R- or S-form

The invention also provides polypeptides that are “substantially identical” to an exemplary polypeptide of the invention. A “substantially identical” amino acid sequence is a sequence that differs from a reference sequence by one or more conservative or non-conservative amino acid substitutions, deletions, or insertions, particularly when such a substitution occurs at a site that is not the active site of the molecule, and provided that the polypeptide essentially retains its functional properties. A conservative amino acid substitution, for example, substitutes one amino acid for another of the same class (e.g., substitution of one hydrophobic amino acid, such as isoleucine, valine, leucine, or methionine, for another, or substitution of one polar amino acid for another, such as substitution of arginine for lysine, glutamic acid for aspartic acid or glutamine for □adioimmuno). One or more amino acids can be deleted, for example, from an αB crystallin polypeptide having molecular chaperone activity of the invention, resulting in modification of the structure of the polypeptide, without significantly altering its biological activity. For example, amino- or carboxyl-terminal, or internal, amino acids which are not required for molecular chaperone activity of αB crystallin protein can be removed.

The skilled artisan will recognize that individual synthetic residues and polypeptides incorporating these mimetics can be synthesized using a variety of procedures and methodologies, which are well described in the scientific and patent literature, e.g., Organic Syntheses Collective Volumes, Gilman, et al. (Eds) John Wiley & Sons, Inc., NY. Peptides and peptide mimetics of the invention can also be synthesized using combinatorial methodologies. Various techniques for generation of peptide and peptidomimetic libraries are well known, and include, e.g., multipin, tea bag, and split-couple-mix techniques; see, e.g., al-Obeidi, Mol. Biotechnol. 9: 205-223, 1998; Hruby, Curr. Opin. Chem. Biol. 1: 114-119, 1997; Ostergaard, Mol. Divers. 3: 17-27, 1997; Ostresh, Methods Enzymol. 267: 220-234, 1996. Modified peptides of the invention can be further produced by chemical modification methods, see, e.g., Belousov, Nucleic Acids Res. 25: 3440-3444, 1997; Frenkel, Free Radic. Biol. Med. 19: 373-380, 1995; Blommers, Biochemistry 33: 7886-7896, 1994.

Peptides and polypeptides of the invention can also be synthesized and expressed as fusion proteins with one or more additional domains linked thereto for, e.g., producing a more immunogenic peptide, to more readily isolate a recombinantly synthesized peptide, to identify and isolate antibodies and antibody-expressing B cells, and the like. Detection and purification facilitating domains include, e.g., metal chelating peptides such as polyhistidine tracts and histidine-tryptophan modules that allow purification on immobilized metals, protein A domains that allow purification on immobilized immunoglobulin, and the domain utilized in the FLAGS extension/affinity purification system (Amgen Corp, Seattle, Wash.). The inclusion of a cleavable linker sequences such as Factor Xa or enterokinase (Invitrogen, San Diego, Calif.) between a purification domain and the motif-comprising peptide or polypeptide to facilitate purification. For example, an expression vector can include an epitope-encoding nucleic acid sequence linked to six histidine residues followed by a thioredoxin and an enterokinase cleavage site (see e.g., Williams, Biochemistry 34: 1787-1797, 1995; Dobeli, Protein Expr. Purif. 12: 404-14, 1998). The histidine residues facilitate detection and purification while the enterokinase cleavage site provides a means for purifying the epitope from the remainder of the fusion protein. Technology pertaining to vectors encoding fusion proteins and application of fusion proteins are well described in the scientific and patent literature, see e.g., Kroll, DNA Cell. Biol., 12: 441-53, 1993.

As used herein, the term “isolated” means that the material is removed from its original environment (e.g., the natural environment if it is naturally occurring). For example, a naturally occurring polynucleotide or polypeptide present in a living animal is not isolated, but the same polynucleotide or polypeptide, separated from some or all of the coexisting materials in the natural system, is isolated. Such polynucleotides could be part of a vector and/or such polynucleotides or polypeptides could be part of a composition, and still be isolated in that such vector or composition is not part of its natural environment. As used herein, an isolated material or composition can also be a “purified” composition, i.e., it does not require absolute purity; rather, it is intended as a relative definition. Individual nucleic acids obtained from a library can be conventionally purified to electrophoretic homogeneity. In alternative aspects, the invention provides nucleic acids which have been purified from genomic DNA or from other sequences in a library or other environment by at least one, two, three, four, five or more orders of magnitude.

Intellipeptide analogs, polypeptide fragment of αB crystallin protein having molecular chaperone activity, are generally designed and produced by chemical modifications of a lead peptide, including, e.g., any of the particular peptides described herein, such as any of the following sequences: i) DPLTITSSLSSDGVLTVNGPRKQ; ii) LTITSSLSDGVLTVNGPRK; iii) LTITSSLSDGV; iv) GPERTIPITREEK; v) PERTIPITREEK; yl) ERTIPITRE; or functional variants or peptide mimetics thereof. An exemplary polypeptide fragment of αB crystallin protein having molecular chaperone activity is presented; e.g., the α crystallin core domain polypeptide fragment is ₁₁₃FISREFHR₁₂₀, or ₁₃₁LTITSSLS₁₃₈ (β8), or the C-terminal domain polypeptide fragment is ₁₅₇RTIPITRE₁₆₄, or functional variants thereof.

The terms “identical” or percent “identity”, in the context of two or more nucleic acids or polypeptide sequences, refers to two or more sequences or subsequences that are the same or have a specified percentage of amino acid residues or nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region (e.g., nucleotide sequence encoding an peptide, peptide analog or peptide mimetic described herein or amino acid sequence described herein), when compared and aligned for maximum correspondence over a comparison window or designated region) as measured using a BLAST or BLAST 2.0 sequence comparison algorithms with default parameters described below, or by manual alignment and visual inspection. Such sequences are then said to be “substantially identical.” This term also refers to, or can be applied to, the compliment of a test sequence. The term also includes sequences that have deletions and/or additions, as well as those that have substitutions. As described below, the preferred algorithms can account for gaps and the like. Preferably, identity exists over a region that is at least about 25 amino acids or nucleotides in length, or more preferably over a region that is 50-100 amino acids or nucleotides in length.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Preferably, default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of any one of the number of contiguous positions selected from the group consisting of from 20 to 600, usually about 50 to about 200, more usually about 100 to about 150 in which a sequence can be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith & Waterman, Adv. Appl. Math. 2: 482, 1981, by the homology alignment algorithm of Needleman & Wunsch, J. Mol. Biol. 48: 443, 1970, by the search for similarity method of Pearson & Lipman, Proc. Nat'l. Acad. Sci. USA 85: 2444, 1988, by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Current Protocols in Molecular Biology, Ausubel et al., eds. 1995 supplement)).

Programs for searching for alignments are well known in the art, e.g., BLAST and the like. For example, if the target species is human, a source of such amino acid sequences or gene sequences can be found in any suitable reference database such as Genbank, the NCBI protein databank (http://ncbi.nlm.nih.gov/BLAST/), VBASE, a database of human antibody genes (http://www.mrc-cpe.cam.ac.uk/imt-doc), and the Kabat database of immunoglobulins (http://www.immuno.bme.nwu.edu) or translated products thereof. If the alignments are done based on the nucleotide sequences, then the selected genes should be analyzed to determine which genes of that subset have the closest amino acid homology to the originating species antibody. It is contemplated that amino acid sequences or gene sequences which approach a higher degree homology as compared to other sequences in the database can be utilized and manipulated in accordance with the procedures described herein. Moreover, amino acid sequences or genes which have lesser homology can be utilized when they encode products which, when manipulated and selected in accordance with the procedures described herein, exhibit specificity for the predetermined target antigen. In certain embodiments, an acceptable range of homology is greater than about 50%. It should be understood that target species can be other than human.

A preferred example of algorithm that is suitable for determining percent sequence identity and sequence similarity are the BLAST and BLAST 2.0 algorithms, which are described in Altschul et al., Nuc. Acids Res. 25: 3389-3402, 1977 and Altschul et al., J. Mol. Biol. 215: 403-410, 1990, respectively. BLAST and BLAST 2.0 are used, with the parameters described herein, to determine percent sequence identity for the nucleic acids and proteins of the invention. Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold. These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4 and a comparison of both strands. For amino acid sequences, the BLASTP program uses as defaults a wordlength of 3, and expectation (E) of 10, and the BLOSUM62 scoring matrix (see Henikoff & Henikoff, Proc. Natl. Acad. Sci. USA 89: 10915, 1989) alignments (B) of 50, expectation (E) of 10, M=5, N=−4, and a comparison of both strands.

“Polypeptide”, “peptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer. “Polypeptide” and “protein” further refer to amino acids joined to each other by peptide bonds or modified peptide bonds, i.e., peptide isosteres, and can contain modified amino acids other than the 20 gene-encoded amino acids. The term “polypeptide” also includes peptides and polypeptide fragments, motifs and the like. The term also includes glycosylated polypeptides. The peptides and polypeptides of the invention also include all “mimetic” and “peptidomimetic” forms, as described in further detail, below.

“Amino acid” or “functional variant or mimetic” of a polypeptide refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an α carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid.

Amino acids can be referred to herein by either their commonly known three letter symbols or by the one-letter symbols recommended by the IUPAC-IUB Biochemical Nomenclature Commission. Nucleotides, likewise, can be referred to by their commonly accepted single-letter codes.

“Conservatively modified variants” or “variants” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations”, which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence with respect to the expression product, but not with respect to actual probe sequences.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention.

The following eight groups each contain amino acids that are conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine (M) (see, e.g., Creighton, Proteins, 1984).

Macromolecular structures such as polypeptide structures can be described in terms of various levels of organization. For a general discussion of this organization, see, e.g., Alberts et al, Molecular Biology of the Cell (3rd ed., 1994) and Cantor and Schimmel, Biophysical Chemistry Part I: The Conformation of Biological Macromolecules, 1980. “Primary structure” refers to the amino acid sequence of a particular peptide. “Secondary structure” refers to locally ordered, three dimensional structures within a polypeptide. These structures are commonly known as domains, e.g., enzymatic domains, extracellular domains, transmembrane domains, pore domains, and cytoplasmic tail domains. Domains are portions of a polypeptide that form a compact unit of the polypeptide and are typically 15 to 350 amino acids long. Exemplary domains include domains with enzymatic activity, e.g., a kinase domain. Typical domains are made up of sections of lesser organization such as stretches of β-sheet and α-helices. “Tertiary structure” refers to the complete three dimensional structure of a polypeptide monomer. “Quaternary structure” refers to the three dimensional structure formed by the noncovalent association of independent tertiary units. Anisotropic terms are also known as energy terms.

A particular nucleic acid sequence also implicitly encompasses “splice variants.” Similarly, a particular protein encoded by a nucleic acid implicitly encompasses any protein encoded by a splice variant of that nucleic acid. “Splice variants”, as the name suggests, are products of alternative splicing of a gene. After transcription, an initial nucleic acid transcript can be spliced such that different (alternate) nucleic acid splice products encode different polypeptides. Mechanisms for the production of splice variants vary, but include alternate splicing of exons. Alternate polypeptides derived from the same nucleic acid by read-through transcription are also encompassed by this definition. Any products of a splicing reaction, including recombinant forms of the splice products, are included in this definition.

Functional variants of polypeptides of these genes and gene products are useful in the invention. “Functional variant” refers to a nucleic acid or protein having a nucleotide sequence or amino acid sequence, respectively, that is “identical,” “essentially identical,” “substantially identical,” “homologous” or “similar” (as described below) to a reference sequence which may, by way of non-limiting example, be the sequence of an isolated nucleic acid or protein, or a consensus sequence derived by comparison of two or more related nucleic acids or proteins, or a group of isoforms of a given nucleic acid or protein. Non-limiting examples of types of isoforms include isoforms of differing molecular weight that result from, e.g., alternate RNA splicing or proteolytic cleavage; and isoforms having different post-translational modifications, such as glycosylation; and the like.

Two sequences are said to be “identical” if the two sequences, when aligned with each other, are exactly the same with no gaps, substitutions, insertions or deletions.

Two sequences are said to be “essentially identical” if the following criteria are met. Two amino acid sequences are “essentially identical” if the two sequences, when aligned with each other, are exactly the same with no gaps, insertions or deletions, and the sequences have only conservative amino acid substitutions. Conservative amino acid substitutions are as described in Table 3.

TABLE 3
CONSERVATIVE AMINO ACID SUBSTITUTIONS

| Type of Amino | Groups of Amino Acids that Are Conservative |
|---|---|
| Acid Side Chain | Substitutions Relative to Each Other |
| Short side chain | Glycine, Alanine, Serine, Threonine and Methionine |
| Hydrophobic | Leucine, Isoleucine and Valine |
| Polar | Glutamine and Asparagine |
| Acidic | Glutamic Acid and Aspartic Acid |
| Basic | Arginine, Lysine and Histidine |
| Aromatic | Phenylalanine, Tryptophan and Tyrosine |

Two nucleotide sequences are “essentially identical” if they encode the identical or essentially identical amino acid sequence. As is known in the art, due to the nature of the genetic code, some amino acids are encoded by several different three base codons, and these codons may thus be substituted for each other without altering the amino acid at that position in an amino acid sequence. In the genetic code, TTA, TTG, CTT, CTC, CTA and CTG encode Leu; AGA, AGG, CGT, CGC, CGA and CGG encode Arg; GCT, GCC, GCA and GCG encode Ala; GGT, GGC, GGA and GGG encode Gly; ACT, ACC, ACA and ACG encode Thr; GTT, GTC, GTA and GTG encode Val; TCT, TCC, TCA and TCG encode Ser; CCT, CCC, CCA and CCG encode Pro; ATA, ATC and ATA encode Ile; GAA and GAG encode Glu; CAA and CAG encode Gln; GAT and GAC encode Asp; AAT and AAC encode Asn; AGT and AGC encode Ser; TAT and TAC encode Tyr; TGT and TGC encode Cys; AAA and AAG encode Lys; CAT and CAC encode His; TTT and TTC encode Phe, TGG encodes Trp; ATG encodes Met; and TGA, TAA and TAG are translation stop codons.

Two amino acid sequences are “substantially identical” if, when aligned, the two sequences are, (i) less than 30%, preferably <20%, more preferably <15%, most preferably <10%, of the identities of the amino acid residues vary between the two sequences; (ii) the number of gaps between or insertions in, deletions of and/or substitutions of, is <10%, more preferably <5%, more preferably <3%, most preferably <1%, of the number of amino acid residues that occur over the length of the shortest of two aligned sequences.

Two sequences are said to be “homologous” if any of the following criteria are met. The term “homolog” includes without limitation orthologs (homologs having genetic similarity as the result of sharing a common ancestor and encoding proteins that have the same function in different species) and paralog (similar to orthologs, yet gene and protein similarity is the result of a gene duplication).

One indication that nucleotide sequences are homologous is if two nucleic acid molecules hybridize to each other under stringent conditions. Stringent conditions are sequence dependent and will be different in different circumstances. Generally, stringent conditions are selected to be about 5° C. lower than the thermal melting point (Tₘ) for the specific sequence at a defined ionic strength and pH. The Tₘ is the temperature (under defined ionic strength and pH) at which 50% of the target sequence hybridizes to a perfectly matched probe. Typically, stringent conditions will be those in which the salt concentration is about 0.02 M at pH 7 and the temperature is at least about 60° C.

Another way by which it can be determined if two sequences are homologous is by using an appropriate algorithm to determine if the above-described criteria for substantially identical sequences are met. Sequence comparisons between two (or more) polynucleotides or polypeptides are typically performed by algorithms such as, for example, the local homology algorithm of Smith and Waterman, Adv. Appl. Math. 2: 482, 1981; by the homology alignment algorithm of Needleman and Wunsch, J. Mol. Biol. 48: 443, 1970; by the search for similarity method of Pearson and Lipman, Proc. Natl. Acad. Sci. U.S.A. 85: 2444, 1988; and by computerized implementations of these algorithms (GAP, BESTFIT, FASTA, and TFASTA in the Wisconsin Genetics Software Package, version 10.2 Genetics Computer Group (GCG), 575 Science Dr., Madison, Wis.); BLASTP, BLASTN, and FASTA (Altschul et al., J. Mol. Biol. 215: 403-410, 1990); or by visual inspection.

Optimal alignments are found by inserting gaps to maximize the number of matches using the local homology algorithm of Smith and Waterman, Adv. Appl. Math. 2: 482-489, 1981. “Gap” uses the algorithm of Needleman and Wunsch, J Mol. Biol. 48: 443-453, 1970) to find the alignment of two complete sequences that maximizes the number of matches and minimizes the number of gaps. In such algorithms, a “penalty” of about 3.0 to about 20 for each gap, and no penalty for end gaps, is used.

Homologous proteins also include members of clusters of orthologous groups of proteins (COGs), which are generated by phylogenetic classification of proteins encoded in complete genomes. To date, COGs have been delineated by comparing protein sequences encoded in 43 complete genomes, representing 30 major phylogenetic lineages. Each COG consists of individual proteins or groups of paralogs from at least 3 lineages and thus corresponds to an ancient conserved domain (see Tatusov et al., Science, 278: 631-637, 1997; Tatusov et al., Nucleic Acids Res. 29: 22-28, 2001; Chervitz et al., Science 282: 2022-2028, 1998; and http://www.ncbi.nlm.nih.gov/COG/).

The entirety of two sequences may be identical, essentially identical, substantially identical, or homologous to one another, or portions of such sequences may be identical or substantially identical with sequences of similar length in other sequences. In either case, such sequences are similar to each other. Typically, stretches of identical or essentially within similar sequences have a length of >12, preferably >24, more preferably >48, and most preferably >96 residues.

“Recombinant” when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, for example, recombinant cells express genes that are not found within the native (non-recombinant) form of the cell or express native genes that are otherwise abnormally expressed, under-expressed or not expressed at all.

The phrase “stringent hybridization conditions” refers to conditions under which a probe will hybridize to its target subsequence, typically in a complex mixture of nucleic acids, but to no other sequences. Stringent conditions are sequence-dependent and will be different in different circumstances. Longer sequences hybridize specifically at higher temperatures. An extensive guide to the hybridization of nucleic acids is found in Tijssen, “Techniques in Biochemistry and Molecular Biology—Hybridization with Nucleic Probes,” Overview of principles of hybridization and the strategy of nucleic acid assays, 1993. Generally, stringent conditions are selected to be about 5-10° C. lower than the thermal melting point (Tₘ) for the specific sequence at a defined ionic strength pH. The Tₘ is the temperature (under defined ionic strength, pH, and nucleic concentration) at which 50% of the probes complementary to the target hybridize to the target sequence at equilibrium (as the target sequences are present in excess, at Tₘ, 50% of the probes are occupied at equilibrium). Stringent conditions can also be achieved with the addition of destabilizing agents such as formamide. For selective or specific hybridization, a positive signal is at least two times background, preferably 10 times background hybridization. Exemplary stringent hybridization conditions can be as following: 50% formamide, 5×SSC, and 1% SDS, incubating at 42° C., or, 5×SSC, 1% SDS, incubating at 65° C., with wash in 0.2×SSC, and 0.1% SDS at 65° C.

Nucleic acids that do not hybridize to each other under stringent conditions are still substantially identical if the polypeptides which they encode are substantially identical. This occurs, for example, when a copy of a nucleic acid is created using the maximum codon degeneracy permitted by the genetic code. In such cases, the nucleic acids typically hybridize under moderately stringent hybridization conditions. Exemplary “moderately stringent hybridization conditions” include a hybridization in a buffer of 40% formamide, 1 M NaCl, 1% SDS at 37° C., and a wash in 1×SSC at 45° C. A positive hybridization is at least twice background. Those of ordinary skill will readily recognize that alternative hybridization and wash conditions can be utilized to provide conditions of similar stringency. Additional guidelines for determining hybridization parameters are provided in numerous reference, e.g., Ausubel et al, supra.

For PCR, a temperature of about 36° C. is typical for low stringency amplification, although annealing temperatures can vary between about 32° C. and 48° C. depending on primer length. For high stringency PCR amplification, a temperature of about 62° C. is typical, although high stringency annealing temperatures can range from about 50° C. to about 65° C., depending on the primer length and specificity. Typical cycle conditions for both high and low stringency amplifications include a denaturation phase of 90° C.-95° C. for 30 sec-2 min., an annealing phase lasting 30 sec.-2 min., and an extension phase of about 72° C. for 1-2 min. Protocols and guidelines for low and high stringency amplification reactions are provided, e.g., in Innis et al., PCR Protocols, A Guide to Methods and Applications, Academic Press, Inc. N.Y., 1990.

### Polypeptides and Functional Variants Thereof

“Polypeptide” includes proteins, fusion proteins, oligopeptides and polypeptide derivatives, with the exception that peptidomimetics are considered to be small molecules herein. Although they are polypeptides, antibodies and their derivatives are described in a separate section. Antibodies and antibody derivatives are described in a separate section, but antibodies and antibody derivatives are, for purposes of the invention, treated as a subclass of the polypeptides and derivatives. Polypeptides include Intellipeptides that interact with tubulin to increase microtubule assembly or inhibit microtubule disassembly. Intellipeptides can also interact with tubulin to decrease thermal aggregation of tubulin, e.g., i) DPLTITSSLSSDGVLTVNGPRKQ; ii) LTITSSLSDGVLTVNGPRK; iii) LTITSSLSDGV; iv) GPERTIPITREEK; v) PERTIPITREEK; vi) ERTIPITRE; or functional variants or peptide mimetics thereof.

A “protein” is a molecule having a sequence of amino acids that are linked to each other in a linear molecule by peptide bonds. The term protein refers to a polypeptide that is isolated from a natural source, or produced from an isolated cDNA using recombinant DNA technology; and has a sequence of amino acids having a length of at least about 200 amino acids.

A “fusion protein” is a type of recombinant protein that has an amino acid sequence that results from the linkage of the amino acid sequences of two or more normally separate polypeptides.

A “protein fragment” is a proteolytic fragment of a larger polypeptide, which may be a protein or a fusion protein. A proteolytic fragment may be prepared by in vivo or in vitro proteolytic cleavage of a larger polypeptide, and is generally too large to be prepared by chemical synthesis. Proteolytic fragments have amino acid sequences having a length from about 200 to about 1,000 amino acids.

An “oligopeptide” is a polypeptide having a short amino acid sequence (i.e., 2 to about 200 amino acids). An oligopeptide is generally prepared by chemical synthesis.

Although oligopeptides and protein fragments may be otherwise prepared, it is possible to use recombinant DNA technology and/or in vitro biochemical manipulations. For example, a nucleic acid encoding an amino acid sequence may be prepared and used as a template for in vitro transcription/translation reactions. In such reactions, an exogenous nucleic acid encoding a preselected polypeptide is introduced into a mixture that is essentially depleted of exogenous nucleic acids that contains all of the cellular components required for transcription and translation. One or more radiolabeled amino acids are added before or with the exogenous DNA, and transcription and translation are allowed to proceed. Because the only nucleic acid present in the reaction mix is the exogenous nucleic acid added to the reaction, only polypeptides encoded thereby are produced, and incorporate the radiolabelled amino acid(s). In this manner, polypeptides encoded by a preselected exogenous nucleic acid are radiolabeled. Although other proteins are present in the reaction mix, the preselected polypeptide is the only one that is produced in the presence of the radiolabeled amino acids and is thus uniquely labeled.

As is explained in detail below, “polypeptide derivatives” include without limitation mutant polypeptides, chemically modified polypeptides, and peptidomimetics.

The polypeptides of this invention, including the analogs and other modified variants, may generally be prepared following known techniques. Preferably, synthetic production of the polypeptide of the invention may be according to the solid phase synthetic method. For example, the solid phase synthesis is well understood and is a common method for preparation of polypeptides, as are a variety of modifications of that technique. Merrifield, J. Am. Chem. Soc., 85: 2149, 1964; Stewart and Young, Solid Phase polypeptide Synthesis, Pierce Chemical Company, Rockford, Ill., 1984; Bodansky and Bodanszky, The Practice of polypeptide Synthesis, Springer-Verlag, New York, 1984; Atherton and Sheppard, Solid Phase polypeptide Synthesis: A Practical Approach, IRL Press, New York, 1989]. See, also, the specific method described in Example 1 below.

Alternatively, polypeptides of this invention may be prepared in recombinant systems using polynucleotide sequences encoding the polypeptides. For example, fusion proteins are typically prepared using recombinant DNA technology.

Functional Polypeptide Variant. A “variant” or “functional variant” of a polypeptide is a compound that is not, by definition, a polypeptide, i.e., it contains at least one chemical linkage that is not a peptide bond. Thus, polypeptide derivatives include without limitation proteins that naturally undergo post-translational modifications such as, e.g., glycosylation. It is understood that a polypeptide of the invention may contain more than one of the following modifications within the same polypeptide. Preferred polypeptide derivatives retain a desirable attribute, which may be biological activity; more preferably, a polypeptide derivative is enhanced with regard to one or more desirable attributes, or has one or more desirable attributes not found in the parent polypeptide. Although they are described in this section, peptidomimetics are taken as small molecules in the present disclosure.

A polypeptide having an amino acid sequence identical to that found in a protein prepared from a natural source is a “wildtype” polypeptide. Functional variants of polypeptides can be prepared by chemical synthesis, including without limitation combinatorial synthesis.

Functional variants of polypeptides larger than oligopeptides can be prepared using recombinant DNA technology by altering the nucleotide sequence of a nucleic acid encoding a polypeptide. Although some alterations in the nucleotide sequence will not alter the amino acid sequence of the polypeptide encoded thereby (“silent” mutations), many will result in a polypeptide having an altered amino acid sequence that is altered relative to the parent sequence. Such altered amino acid sequences may comprise substitutions, deletions and additions of amino acids, with the proviso that such amino acids are naturally occurring amino acids.

Thus, subjecting a nucleic acid that encodes a polypeptide to mutagenesis is one technique that can be used to prepare Functional variants of polypeptides, particularly ones having substitutions of amino acids but no deletions or insertions thereof. A variety of mutagenic techniques are known that can be used in vitro or in vivo including without limitation chemical mutagenesis and PCR-mediated mutagenesis. Such mutagenesis may be randomly targeted (i.e., mutations may occur anywhere within the nucleic acid) or directed to a section of the nucleic acid that encodes a stretch of amino acids of particular interest. Using such techniques, it is possible to prepare randomized, combinatorial or focused compound libraries, pools and mixtures.

Polypeptides having deletions or insertions of naturally occurring amino acids may be synthetic oligopeptides that result from the chemical synthesis of amino acid sequences that are based on the amino acid sequence of a parent polypeptide but which have one or more amino acids inserted or deleted relative to the sequence of the parent polypeptide. Insertions and deletions of amino acid residues in polypeptides having longer amino acid sequences may be prepared by directed mutagenesis.

Chemically Modified Polypeptides. As contemplated by this invention, “polypeptide” includes those having one or more chemical modification relative to another polypeptide, i.e., chemically modified polypeptides. The polypeptide from which a chemically modified polypeptide is derived may be a wildtype protein, a functional variant protein or a functional variant polypeptide, or polypeptide fragments thereof; an antibody or other polypeptide ligand according to the invention including without limitation single-chain antibodies, crystalline proteins and polypeptide derivatives thereof; or polypeptide ligands prepared according to the disclosure. Preferably, the chemical modification(s) confer(s) or improve(s) desirable attributes of the polypeptide but does not substantially alter or compromise the biological activity thereof. Desirable attributes include but are limited to increased shelf-life; enhanced serum or other in vivo stability; resistance to proteases; and the like. Such modifications include by way of non-limiting example N-terminal acetylation, glycosylation, and biotinylation.

Polypeptides with N-Terminal or C-Terminal Chemical Groups. An effective approach to confer resistance to peptidases acting on the N-terminal or C-terminal residues of a polypeptide is to add chemical groups at the polypeptide termini, such that the modified polypeptide is no longer a substrate for the peptidase. One such chemical modification is glycosylation of the polypeptides at either or both termini. Certain chemical modifications, in particular N-terminal glycosylation, have been shown to increase the stability of polypeptides in human serum (Powell et al., Pharma. Res. 10: 1268-1273, 1993). Other chemical modifications which enhance serum stability include, but are not limited to, the addition of an N-terminal alkyl group, consisting of a lower alkyl of from 1 to 20 carbons, such as an acetyl group, and/or the addition of a C-terminal amide or substituted amide group.

Polypeptides with a Terminal D-Amino Acid. The presence of an N-terminal D-amino acid increases the serum stability of a polypeptide that otherwise contains L-amino acids, because exopeptidases acting on the N-terminal residue cannot utilize a D-amino acid as a substrate. Similarly, the presence of a C-terminal D-amino acid also stabilizes a polypeptide, because serum exopeptidases acting on the C-terminal residue cannot utilize a D-amino acid as a substrate. With the exception of these terminal modifications, the amino acid sequences of polypeptides with N-terminal and/or C-terminal D-amino acids are usually identical to the sequences of the parent L-amino acid polypeptide.

Polypeptides with Substitution of Natural Amino Acids by Unnatural Amino Acids. Substitution of unnatural amino acids for natural amino acids in a subsequence of a polypeptide can confer or enhance desirable attributes including biological activity. Such a substitution can, for example, confer resistance to proteolysis by exopeptidases acting on the N-terminus. The synthesis of polypeptides with unnatural amino acids is routine and known in the art (see, for example, Coller, et al. 1993, cited above).

Post-Translational Chemical Modifications. Different host cells will contain different post-translational modification mechanisms that may provide particular types of post-translational modification of a fusion protein if the amino acid sequences required for such modifications is present in the fusion protein. A large number (.about. 100) of post-translational modifications have been described, a few of which are discussed herein. One skilled in the art will be able to choose appropriate host cells, and design chimeric genes that encode protein members comprising the amino acid sequence needed for a particular type of modification.

Glycosylation is one type of post-translational chemical modification that occurs in many eukaryotic systems, and may influence the activity, stability, pharmacogenetics, immunogenicity and/or antigenicity of proteins. However, specific amino acids must be present at such sites to recruit the appropriate glycosylation machinery, and not all host cells have the appropriate molecular machinery. Saccharomyces cerevisieae and Pichia pastoris provide for the production of glycosylated proteins, as do expression systems that utilize insect cells, although the pattern of glyscoylation may vary depending on which host cells are used to produce the fusion protein.

Another type of post-translation modification is the phosphorylation of a free hydroxyl group of the side chain of one or more Ser, Thr or Tyr residues, Protein kinases catalyze such reactions. Phosphorylation is often reversible due to the action of a protein phosphatase, an enzyme that catalyzes the dephosphorylation of amino acid residues.

Differences in the chemical structure of amino terminal residues result from different host cells, each of which may have a different chemical version of the methionine residue encoded by a start codon, and these will result in amino termini with different chemical modifications.

For example, many or most bacterial proteins are synthesized with an amino terminal amino acid that is a modified form of methionine, i.e, N-formyl-methionine (fMet). Although the statement is often made that all bacterial proteins are synthesized with an fMet initiator amino acid; although this may be true for E. coli, recent studies have shown that it is not true in the case of other bacteria such as Pseudomonas aeruginosa (Newton et al., J. Biol. Chem. 274: 22143-22146, 1999). In any event, in E. coli, the formyl group of fMet is usually enzymatically removed after translation to yield an amino terminal methionine residue, although the entire fMet residue is sometimes removed (see Hershey, Chapter 40, “Protein Synthesis” in: Escherichia coli and Salmonella typhimurium: Cellular and Molecular Biology, Neidhardt, Frederick C., Editor in Chief, American Society for Microbiology, Washington, D.C., 1987, Volume 1, pages 613-647, and references cited therein.) E. coli mutants that lack the enzymes (such as, e.g., formylase) that catalyze such post-translational modifications will produce proteins having an amino terminal fMet residue (Guillon et al., J. Bacteriol. 174: 4294-4301, 1992).

In eukaryotes, acetylation of the initiator methionine residue, or the penultimate residue if the initiator methionine has been removed, typically occurs co- or post-translationally. The acetylation reactions are catalyzed by N-terminal acetyltransferases (NATs, a.k.a. N-alpha-acetyltransferases), whereas removal of the initiator methionine residue is catalyzed by methionine aminopeptidases (for reviews, see Bradshaw et al., Trends Biochem. Sci. 23: 263-267, 1998; and Driessen et al., CRC Crit. Rev. Biochem. 18: 281-325, 1985). Amino terminally acetylated proteins are said to be “N-acetylated,” “N alpha acetylated” or simply “acetylated.”

Another post-translational process that occurs in eukaryotes is the alpha-amidation of the carboxy terminus. For reviews, see Eipper et al. Annu. Rev. Physiol. 50: 333-344, 1988, and Bradbury et al. Lung Cancer 14: 239-251, 1996. About 50% of known endocrine and neuroendocrine peptide hormones are alpha-amidated (Treston et al., Cell Growth Differ. 4: 911-920, 1993). In most cases, carboxy alpha-amidation is required to activate these peptide hormones.

### Polypeptide Mimetic

In general, a polypeptide mimetic (“peptidomimetic”) is a molecule that mimics the biological activity of a polypeptide but is no longer peptidic in chemical nature. By strict definition, a peptidomimetic is a molecule that contains no peptide bonds (that is, amide bonds between amino acids). However, the term peptidomimetic is sometimes used to describe molecules that are no longer completely peptidic in nature, such as pseudo-peptides, semi-peptides and peptoids. Examples of some peptidomimetics by the broader definition (where part of a polypeptide is replaced by a structure lacking peptide bonds) are described below. Whether completely or partially non-peptide, peptidomimetics according to this invention provide a spatial arrangement of reactive chemical moieties that closely resembles the three-dimensional arrangement of active groups in the polypeptide on which the peptidomimetic is based. As a result of this similar active-site geometry, the peptidomimetic has effects on biological systems that are similar to the biological activity of the polypeptide. Polypeptide mimetics include Intellipeptides that interact with tubulin to increase microtubule assembly or inhibit microtubule disassembly. Intellipeptides can also interact with tubulin to decrease thermal aggregation of tubulin.

There are several potential advantages for using a mimetic of a given polypeptide rather than the polypeptide itself. For example, polypeptides may exhibit two undesirable attributes, i.e., poor bioavailability and short duration of action. Peptidomimetics are often small enough to be both orally active and to have a long duration of action. There are also problems associated with stability, storage and immunoreactivity for polypeptides that are not experienced with peptidomimetics.

Candidate, lead and other polypeptides having a desired biological activity can be used in the development of peptidomimetics with similar biological activities. Techniques of developing peptidomimetics from polypeptides are known. Peptide bonds can be replaced by non-peptide bonds that allow the peptidomimetic to adopt a similar structure, and therefore biological activity, to the original polypeptide. Further modifications can also be made by replacing chemical groups of the amino acids with other chemical groups of similar structure. The development of peptidomimetics can be aided by determining the tertiary structure of the original polypeptide, either free or bound to a ligand, by NMR spectroscopy, crystallography and/or computer-aided molecular modeling. These techniques aid in the development of novel compositions of higher potency and/or greater bioavailability and/or greater stability than the original polypeptide (Dean, BioEssays, 16: 683-687, 1994; Cohen and Shatzmiller, J. Mol. Graph., 11: 166-173, 1993; Wiley and Rich, Med. Res. Rev., 13: 327-384, 1993; Moore, Trends Pharmacol. Sci., 15: 124-129, 1994; Hruby, Biopolymers, 33: 1073-1082, 1993; Bugg et al., Sci. Am., 269: 92-98, 1993, all incorporated herein by reference].

Thus, through use of the methods described above, the present invention provides compounds exhibiting enhanced therapeutic activity in comparison to the polypeptides in methods for the treatment of diseases and disorders associated with microtubule assembly and the ability to induce apoptosis in cells in the mammalian subject, e.g., neoplastic diseases, cancer, or solid tumors. The peptidomimetic compounds obtained by the above methods, having the biological activity of the above named polypeptides and similar three-dimensional structure, are encompassed by this invention. It will be readily apparent to one skilled in the art that a peptidomimetic can be generated from any of the modified polypeptides described in the previous section or from a polypeptide bearing more than one of the modifications described from the previous section. It will furthermore be apparent that the peptidomimetics of this invention can be further used for the development of even more potent non-peptidic compounds, in addition to their utility as therapeutic compounds.

Specific examples of peptidomimetics derived from the polypeptides described in the previous section are presented below. These examples are illustrative and not limiting in terms of the other or additional modifications.

Peptides with a Reduced Isostere Pseudopeptide Bond. Proteases act on peptide bonds. It therefore follows that substitution of peptide bonds by pseudopeptide bonds confers resistance to proteolysis. A number of pseudopeptide bonds have been described that in general do not affect polypeptide structure and biological activity. The reduced isostere pseudopeptide bond is a suitable pseudopeptide bond that is known to enhance stability to enzymatic cleavage with no or little loss of biological activity (Couder, et al., Int. J. Polypeptide Protein Res. 41: 181-184, 1993, incorporated herein by reference). Thus, the amino acid sequences of these compounds may be identical to the sequences of their parent L-amino acid polypeptides, except that one or more of the peptide bonds are replaced by an isostere pseudopeptide bond. Preferably the most N-terminal peptide bond is substituted, since such a substitution would confer resistance to proteolysis by exopeptidases acting on the N-terminus.

Peptides with a Retro-Inverso Pseudopeptide Bond. To confer resistance to proteolysis, peptide bonds may also be substituted by retro-inverso pseudopeptide bonds (Dalpozzo, et al., Int. J. Polypeptide Protein Res. 41: 561-566, incorporated herein by reference). According to this modification, the amino acid sequences of the compounds may be identical to the sequences of their L-amino acid parent polypeptides, except that one or more of the peptide bonds are replaced by a retro-inverso pseudopeptide bond. Preferably the most N-terminal peptide bond is substituted, since such a substitution will confer resistance to proteolysis by exopeptidases acting on the N-terminus.

Peptoid Derivatives. Peptoid derivatives of polypeptides represent another form of modified polypeptides that retain the important structural determinants for biological activity, yet eliminate the peptide bonds, thereby conferring resistance to proteolysis (Simon, et al., Proc. Natl. Acad. Sci. USA, 89: 9367-9371, 1992, and incorporated herein by reference). Peptoids are oligomers of N-substituted glycines. A number of N-alkyl groups have been described, each corresponding to the side chain of a natural amino acid.

### Combinatorial Protein Design

The variants typically exhibit the same qualitative biological activity, however the chaperone activity may be altered from that of the original candidate variant protein, as needed. The ability of the variant polypeptide may be altered in its interaction with tubulin to promote microtubule assembly, inhibit microtubule disassembly, or decrease thermal aggregation of tubulin, and therein induce apoptosis in cells in a mammalian subject include, but are not limited to, treatment of neoplastic diseases, cancer, and solid tumors. Alternatively, the variant may be designed such that the biological activity of the candidate variant protein is altered. For example, glycosylation sites may be altered or removed. Similarly, the biological function may be altered.

In addition, in some embodiments, it is desirable to have candidate variant proteins with altered chaperone activity or interaction with tubulin that will bind to the target protein. Preferably, it would be desirable have proteins that exhibit oxidative stability, alkaline stability, and thermal stability.

A change in oxidative stability is evidenced by at least about 20%, more preferably at least about 50% increase of activity of a variant protein when exposed to various oxidizing conditions as compared to that of wild-type protein. Oxidative stability is measured by known procedures.

A change in alkaline stability is evidenced by at least about a 5% or greater increase or decrease (preferably increase) in the half life of the activity of a variant protein when exposed to increasing or decreasing pH conditions as compared to that of wild-type protein. Generally, alkaline stability is measured by known procedures.

A change in thermal stability is evidenced by at least about a 5% or greater increase or decrease (preferably increase) in the half-life of the activity of a variant protein when exposed to a relatively high temperature and neutral pH as compared to that of wild-type protein. Generally, thermal stability is measured by known procedures.

The candidate variant proteins and nucleic acids of the invention can be made in a number of ways. Individual nucleic acids and proteins can be made as known in the art and outlined below. Alternatively, libraries of candidate variant proteins can be made for testing.

In a preferred embodiment, the library of candidate variant proteins is generated from a probability distribution table. As outlined herein, there are a variety of methods of generating a probability distribution table, including using PDA™ technology, sequence alignments, forcefield calculations such as self-consistent meant field (SCMF) calculations. In addition, the probability distribution can be used to generate information entropy scores for each position, as a measure of the mutational frequency observed in the library.

In this embodiment, the frequency of each amino acid residue at each variable position in the list is identified. Frequencies can be thresholded, wherein any variant frequency lower than a cutoff is set to zero. This cutoff is preferably about 1%, 2%, 5%, 10% or 20%, with about 10% being particularly preferred. These frequencies are then built into the library of candidate variant proteins. That is, as above, these variable positions are collected and all possible combinations are generated, but the amino acid residues that “fill” the library of candidate variant proteins are utilized on a frequency basis. Thus, in a non-frequency based library of candidate variant proteins, a variable position that has 5 possible residues will have about 20% of the proteins comprising that variable position with the first possible residue, 20% with the second, etc. However, in a frequency based library of candidate variant proteins, a variable position that has 5 possible residues with frequencies of about 10%, 15%, 25%, 30% and 20%, respectively, will have 10% of the proteins comprising that variable position with the first possible residue, 15% of the proteins with the second residue, 25% with the third, etc. As will be appreciated by those in the art, the actual frequency may depend on the method used to actually generate the proteins; for example, exact frequencies may be possible when the proteins are synthesized. However, when the frequency-based primer system outlined below is used, the actual frequencies at each position will vary, as outlined below.

As will be appreciated by those in the art and outlined herein, probability distribution tables can be generated in a variety of ways. In addition to the methods outlined herein, self-consistent mean field (SCMF) methods can be used in the direct generation of probability tables. SCMF is a deterministic computational method that uses a mean field description of rotamer interactions to calculate energies. A probability table generated in this way can be used to create libraries of candidate variant proteins as described herein. SCMF can be used in three ways: the frequencies of amino acids and rotamers for each amino acid are listed at each position; the probabilities are determined directly from SCMF (see Delarue et al. Pac. Symp. Biocomput. 109-21, 1997, expressly incorporated by reference). In addition, highly variable positions and non-variable positions can be identified. Alternatively, another method is used to determine what sequence is jumped to during a search of sequence space; SCMF is used to obtain an accurate energy for that sequence; this energy is then used to rank it and create a rank-ordered list of sequences (similar to a Monte Carlo sequence list). A probability table showing the frequencies of amino acids at each position can then be calculated from this list. Koehl et al., J. Mol. Biol. 239: 249, 1994; Koehl et al., Nat. Struc. Biol. 2: 163, 1995; Koehl et al., Curr. Opin. Struct. Biol. 6: 222, 1996; Koehl et al., J. Mol. Bio. 293: 1183, 1999; Koehl et al., J. Mol. Biol. 293: 1161, 1999; Lee, J. Mol. Biol. 236: 918, 1994; and Vasquez Biopolymers 36: 53-70, 1995; all of which are expressly incorporated by reference. Similar methods include, but are not limited to, OPLS-AA (Jorgensen, et al., J. Am. Chem. Soc., 118: 11225-11236, 1996; Jorgensen, W. L.; BOSS, Version 4.1; Yale University: New Haven, Conn., 1999); OPLS (Jorgensen, et al., J. Am. Chem. Soc., 110: 1657ff, 1988; Jorgensen, et al., J. Am. Chem. Soc. 112: 4768ff, 1990); UNRES (United Residue Forcefield; Liwo, et al., Protein Science, 2: 1697-1714, 1993; Liwo, et al., Protein Science, 2: 1715-1731, 1993; Liwo, et al., J. Comp. Chem. 18: 849-873, 1997; Liwo, et al., J. Comp. Chem., 18: 874-884, 1997; Liwo, et al., J. Comp. Chem. 19: 259-276, 1998; Forcefield for Protein Structure Prediction (Liwo, et al., Proc. Natl. Acad. Sci. USA, 96: 5482-5485, 1999); ECEPP/3 (Liwo et al., J Protein Chem 13(4): 375-80, 1994); AMBER 1.1 force field (Weiner, et al., J. Am. Chem. Soc. 106: 765-784, 1994); AMBER 3.0 force field (U. C. Singh et al., Proc. Natl. Acad. Sci. USA. 82: 755-759, 1994); CHARMM and CHARMM22 (Brooks, et al., J. Comp. Chem. 4: 187-217); cvff3.0 (Dauber-Osguthorpe, et al., Proteins: Structure, Function and Genetics, 4: 31-47, 1988); cff91 (Maple, et al., J. Comp. Chem. 15: 162-182, 1988); also, the DISCOVER (cvff and cff91) and AMBER forcefields are used in the INSIGHT molecular modeling package (Biosym/MSI, San Diego Calif.) and HARMM is used in the QUANTA molecular modeling package (Biosym/MSI, San Diego Calif.); all references hereby expressly incorporated by reference in their entirety.

In addition, a method of generating a probability distribution table is through the use of sequence alignment programs. In addition, the probability table can be obtained by a combination of sequence alignments and computational approaches. For example, one can add amino acids found in the alignment of homologous sequences to the result of the computation. Preferable one can add the wild type amino acid identity to the probability table if it is not found in the computation.

As will be appreciated, a library of candidate variant proteins created by recombining variable positions and/or residues at the variable position may not be in a rank-ordered list. In some embodiments, the entire list may just be made and tested. Alternatively, in a preferred embodiment, the secondary library is also in the form of a rank ordered list. This may be done for several reasons, including the size of the secondary library is still too big to generate experimentally, or for predictive purposes. This may be done in several ways. In one embodiment, the secondary library is ranked or filtered using the scoring functions of PDA™ to rank or filter the library members. Alternatively, statistical methods could be used. For example, the secondary library may be ranked or filtered by frequency score; that is, proteins containing the most of high frequency residues could be ranked higher, etc. This may be done by adding or multiplying the frequency at each variable position to generate a numerical score. Similarly, the secondary library different positions could be weighted and then the proteins scored; for example, those containing certain residues could be arbitrarily ranked or filtered.

In a one embodiment, the different protein members of the candidate variant library may be chemically synthesized. This is particularly useful when the designed proteins are short, preferably less than 150 amino acids in length, with less than 100 amino acids being preferred, and less than 50 amino acids being particularly preferred, although as is known in the art, longer proteins can be made chemically or enzymatically. See for example Wilken et al., Curr. Opin. Biotechnol. 9: 412-26, 1998, hereby expressly incorporated by reference.

In another embodiment, particularly for longer proteins or proteins for which large samples are desired, the candidate variant sequences are used to create nucleic acids such as DNA which encode the member sequences and which can then be cloned into host cells, expressed and assayed, if desired. Thus, nucleic acids, and particularly DNA, can be made which encodes each member protein sequence. This is done using well known procedures. The choice of codons, suitable expression vectors and suitable host cells will vary depending on a number of factors, and can be easily optimized as needed.

In a further embodiment, multiple PCR reactions with pooled oligonucleotides is done. In this embodiment, overlapping oligonucleotides are synthesized which correspond to the full length gene. Again, these oligonucleotides may represent all of the different amino acids at each variant position or subsets. These oligonucleotides can be pooled in equal proportions and multiple PCR reactions are performed to create full length sequences containing the combinations of mutations defined by the secondary library. In addition, this may be done using error-prone PCR methods. The different oligonucleotides can be added in relative amounts corresponding to the probability distribution table. The multiple PCR reactions thus result in full length sequences with the desired combinations of mutation in the desired proportions.

The total number of oligonucleotides needed is a function of the number of positions being mutated and the number of mutations being considered at these positions: (number of oligos for constant positions)+M₁+M₂+M₃+ . . . Mₙ=(total number of oligos required), where Mₙ, is the number of mutations considered at position n in the sequence.

In a further aspect, each overlapping oligonucleotide comprises only one position to be varied; in alternate embodiments, the variant positions are too close together to allow this and multiple variants per oligonucleotide are used to allow complete recombination of all the possibilities. That is, each oligo can contain the codon for a single position being mutated, or for more than one position being mutated. The multiple positions being mutated must be close in sequence to prevent the oligo length from being impractical. For multiple mutating positions on an oligonucleotide, particular combinations of mutations can be included or excluded in the library by including or excluding the oligonucleotide encoding that combination. For example, as discussed herein, there may be correlations between variable regions; that is, when position X is a certain residue, position Y must (or must not) be a particular residue. These sets of variable positions are sometimes referred to herein as a “cluster”. When the clusters are comprised of residues close together, and thus can reside on one oligonucleotide primer, the clusters can be set to the “good” correlations, and eliminate the bad combinations that may decrease the effectiveness of the library. However, if the residues of the cluster are far apart in sequence, and thus will reside on different oligonucleotides for synthesis, it may be desirable to either set the residues to the “good” correlation, or eliminate them as variable residues entirely. In an alternative embodiment, the library may be generated in several steps, so that the cluster mutations only appear together. This procedure, i.e., the procedure of identifying mutation clusters and either placing them on the same oligonucleotides or eliminating them from the library or library generation in several steps preserving clusters, can considerably enrich the experimental library with properly folded protein. Identification of clusters can be carried out by a number of ways, e.g. by using known pattern recognition methods, comparisons of frequencies of occurrence of mutations or by using energy analysis of the sequences to be experimentally generated (for example, if the energy of interaction is high, the positions are correlated). These correlations may be positional correlations (e.g. variable positions 1 and 2 always change together or never change together) or sequence correlations (e.g. if there is a residue A at position 1, there is always residue B at position 2). See: Pattern discovery in Biomolecular Data: Tools, Techniques, and Applications; edited by Jason T. L. Wang, Bruce A. Shapiro, Dennis Shasha. New York: Oxford University, 1999; Andrews, Harry C. Introduction to mathematical techniques in patter recognition; New York, Wiley-Interscience, 1972; Applications of Pattern Recognition; Editor, K. S. Fu. Boca Raton, Fla. CRC Press, 1982; Genetic Algorithms for Pattern Recognition; edited by Sankar K. Pal, Paul P. Wang. Boca Raton: CRC Press, c1996; Pandya, Abhijit S., Pattern recognition with Neural networks in C++/Abhijit S. Pandya, Robert B. Macy. Boca Raton, Fla.: CRC Press, 1996; Handbook of pattern recognition and computer vision/edited by C. H. Chen, L. F. Pau, P. S. P. Wang. 2nd ed. Signapore; River Edge, N.J.: World Scientific, c1999; Friedman, Introduction to Pattern Recognition: Statistical, Structural, Neural, and Fuzzy Logic Approaches; River Edge, N.J.: World Scientific, c1999, Series title: Serien a machine perception and artificial intelligence; vol. 32; all of which are expressly incorporated by reference. In addition programs used to search for consensus motifs can be used as well.

In addition, correlations and shuffling can be fixed or optimized by altering the design of the oligonucleotides; that is, by deciding where the oligonucleotides (primers) start and stop (e.g. where the sequences are “cut”). The start and stop sites of oligos can be set to maximize the number of clusters that appear in single oligonucleotides, thereby enriching the library with higher scoring sequences. Different oligonucleotides start and stop site options can be computationally modeled and ranked or filtered according to number of clusters that are represented on single oligos, or the percentage of the resulting sequences consistent with the predicted library of sequences.

The total number of oligonucleotides required increases when multiple mutable positions are encoded by a single oligonucleotide. The annealed regions are the ones that remain constant, i.e. have the sequence of the reference sequence.

Oligonucleotides with insertions or deletions of codons can be used to create a library expressing different length proteins. In particular computational sequence screening for insertions or deletions can result in secondary libraries defining different length proteins, which can be expressed by a library of pooled oligonucleotide of different lengths.

In a further aspect, the secondary library is done by shuffling the family (e.g. a set of variants); that is, some set of the top sequences (if a rank-ordered list is used) can be shuffled, either with or without error-prone PCR. “Shuffling” in this context means a recombination of related sequences, generally in a random way. It can include “shuffling” as defined and exemplified in U.S. Pat. Nos. 5,830,721; 5,811,238; 5,605,793; 5,837,458 and PCT US/19256, all of which are expressly incorporated by reference in their entirety. This set of sequences can also be an artificial set; for example, from a probability table (for example generated using SCMF) or a Monte Carlo set. Similarly, the “family” can be the top 10 and the bottom 10 sequences, the top 100 sequences, etc. This may also be done using error-prone PCR.

Thus, in a further aspect, in silico shuffling is done using the computational methods described therein. That is, starting with either two libraries or two sequences, random recombinations of the sequences can be generated and evaluated.

Error-prone PCR can be done to generate the secondary library. See U.S. Pat. Nos. 5,605,793, 5,811,238, and 5,830,721, all of which are hereby incorporated by reference. This can be done on the optimal sequence or on top members of the library, or some other artificial set or family. In this embodiment, the gene for the optimal sequence found in the computational screen of the primary library can be synthesized. Error prone PCR is then performed on the optimal sequence gene in the presence of oligonucleotides that code for the mutations at the variant positions of the secondary library (bias oligonucleotides). The addition of the oligonucleotides will create a bias favoring the incorporation of the mutations in the secondary library. Alternatively, only oligonucleotides for certain mutations may be used to bias the library.

Gene shuffling with error prone PCR can be performed on the gene for the optimal sequence, in the presence of bias oligonucleotides, to create a DNA sequence library that reflects the proportion of the mutations found in the secondary library. The choice of the bias oligonucleotides can be done in a variety of ways; they can chosen on the basis of their frequency, i.e. oligonucleotides encoding high mutational frequency positions can be used; alternatively, oligonucleotides containing the most variable positions can be used, such that the diversity is increased; if the secondary library is ranked or filtered, some number of top scoring positions can be used to generate bias oligonucleotides; random positions may be chosen; a few top scoring and a few low scoring ones may be chosen; etc. What is important is to generate new sequences based on preferred variable positions and sequences.

PCR using a wild type gene or polypeptide sequence can be used. In this embodiment, a starting gene is used; generally, although this is not required, the gene is the wild type gene. In some cases it may be the gene encoding the global optimized sequence, or any other sequence of the list. In this embodiment, oligonucleotides are used that correspond to the variant positions and contain the different amino acids of the secondary library. PCR is done using PCR primers at the termini, as is known in the art. This provides two benefits; the first is that this generally requires fewer oligonucleotides and can result in fewer errors. In addition, it has experimental advantages in that if the wild type gene is used, it need not be synthesized. Ligation of PCR products can be done.

A variety of additional steps may be done to one or more candidate variant secondary libraries; for example, further computational processing can occur, candidate variant secondary libraries can be recombined, or cutoffs from different candidate variant secondary libraries can be combined. In a preferred embodiment, a candidate variant secondary library may be computationally remanipulated to form an additional secondary library (sometimes referred to herein as “tertiary libraries”). For example, any of the candidate variant secondary library sequences may be chosen for a second round of PDA™, by freezing or fixing some or all of the changed positions in the first secondary library. Alternatively, only changes seen in the last probability distribution table are allowed. Alternatively, the stringency of the probability table may be altered, either by increasing or decreasing the cutoff for inclusion. Similarly, the candidate variant secondary library may be recombined experimentally after the first round; for example, the best gene/genes from the first screen may be taken and gene assembly redone (for example, using techniques outlined below, multiple PCR, error prone PCR, or shuffling). Alternatively, the fragments from one or more good gene(s) to change probabilities at some positions. This biases the search to an area of sequence space found in the first round of computational and experimental screening.

### Apoptosis Cell Assay

Measurement of cancer cell inhibition may be done, for example, by means of an apoptosis assay, where an increase in the level of apoptosis indicates that the molecule introduced into the cell system inhibits the cancer cells. Measurement of inhibition may also be done by means of an assay that measures cell proliferation, where a decrease in the rate of cell division indicates that the molecule inhibits the cancer cells. In addition, measurement of cancer cell inhibition may also be done by assessing reduction in the growth of cancer cells in liquid media (anchorage dependent growth) and/or in soft agar (anchorage independent growth), where a measurable decrease in the rate of growth indicates that the molecule inhibits the cancer cells.

An example of an apoptosis assay is the Annexin-V binding assay. This assay is based on the relocation of phosphatidylserine to the outer cell membrane. Viable cells maintain an asymmetric distribution of different phospholipids between the inner and outer leaflets of the plasma membrane. Choline-containing phospholipids such as phosphatidylcholine and sphingomyelin are primarily located on the outer leaflet of viable cells and aminophospholipids such as phosphatidylethanolamine and phosphatidylserine (PS) are found at the cytoplasmic (inner) face of viable cells. The distribution of phospholipids in the plasma membrane changes during apoptosis. In particular, PS relocates from the cytoplasmic face to the outer leaflet so called PS exposure. The extent of PS exposure can distinguish apoptotic cells from the non-apoptotic cells.

Annexin-V is a 35-36 kDa calcium-dependent phospholipid binding protein with high affinity for PS (kDa.about.5.times.10-10 M). When labeled with a fluorescent dye, Annexin-V can be used as a sensitive probe for PS exposure on the outer leaflet of the cell membrane. The binding of Annexin-V conjugates such as Annexin-V FITC to cells permits differentiation of apoptotic cells (Annexin-V positive) from non-apoptotic cells (Annexin-V negative). Annexin-V binding is observed under two conditions. The first condition is observed in cells midway through the apoptosis pathway. Phosphatidylserine translocates to the outer leaflet of the cell membrane. The second condition is observed in very late apoptosis or when the cells become necrotic and membrane permeabilization occurs. This membrane permeabilization allows Annexin-V to enter cells and bind to phosphatidylserine on the cytoplasmic face of the membrane. Since other causes besides apoptosis can result in necrosis, it is important to distinguish between necrotic and apoptotic cells. Membrane permeabilization also permits entry of other materials to the interior of the cell, including the fluorescent DNA-binding dye propidium iodide. Utilizing dual staining methodology, apoptotic populations can be distinguished from necrotic populations. For example, using the Annexin V-propidium iodide (PI) double staining regime, three populations of cells are distinguishable in two color flow cytometry. See Boersma, et al., Cytometry, 24: 123-130, 1996; Martin, et al., J. Exp. Med., 182: 1545-1556, 1995.

Another example of an apoptosis assay is the caspase 3/7 assay. Briefly, caspases are synthesized as inactive pro-enzymes or pro-caspases. In apoptosis, the pro-caspases are processed by proteolytic cleavage to form active enzymes. For example, caspase-3 exists in cells as an inactive 32 kDa proenzyme, called pro-caspase-3. Pro-caspase-3 is cleaved into active 17 and 12 kDa subunits by upstream proteases to become active caspase-3. Caspases-2, -8, -9 and -10 are classified as signaling or “upstream” in the apoptosis pathway because long prodomains allow association with cell surface receptors such as FAS (CD95), TNFR-1 (CD120a), DR-3 or CARD domains. This observation suggests a proteolytic cascade as a mechanism for signaling. A proteolytic cascade exists that would activate the terminal event required for apoptosis in a way similar to that of the coagulation cascade seen with the closely related family of serine proteases. For example, caspase-4 activates pro-caspase-1; caspase-9 activates pro-caspase-3; and caspase-3 cleaves pro-caspase-6 and pro-caspase-7. Caspases play a critical role in the execution phase of apoptosis. Important targets of caspases include cytoplasmic and nuclear proteins such as keratin 18, poly ADP ribose polymerase (PARP) and lamins. Overexpression of caspase-3 induces apoptosis. Through the use of synthetic peptides, caspases have been divided into three groups based on the four amino acids amino-terminal to their cleavage site. Caspases-1, -4 and -5 prefer substrates containing the sequence WEXD (where X is variable). Caspases-2, -3 and -7 prefer the sequence DEXD. Caspases 6, 8 and 9 are the least demanding but have demonstrated a preference for cleaving of substrates containing either LEXD or VEXD. Because these sequences correspond to known cleavage sites of caspase targets, systems to study caspase cleavage activity have been developed. The measurement of caspase enzyme activity with fluorometric and colorimetric peptide substrates and the detection of caspase cleavage using antibodies to caspases allows the study of the apoptosis processes or screening of therapeutic agents which promote or prevent apoptosis. A typical assay would involve the cleavage of a fluorescent substrate peptide to quantitate activity. The substrate, DEVD-AFC, is composed of the fluorophore, AFC (7-amino-4-trifluoromethyl coumarin), and a synthetic tetrapeptide, DEVD (Asp-Glu-Val-Asp), which is the upstream amino acid sequence of the Caspase-3 cleavage site in PARP. DEVD-AFC emits blue light (max=400 nm). Upon cleavage of the substrate by Caspase-3 or related caspases, with the excitation wavelength set to 400 nm, free AFC emits a yellow-green fluorescence (max=505 nm) which can be quantified using a spectrofluorometer or a fluorescence microtiter plate reader. Comparison of the fluorescence of AFC from apoptotic samples with an uninduced control allows determination of the increase in caspase-3 activity. See Jaeschke, et al. J. Immunol., 160: 3480-3486 (1998); Talanian, et al., J. Biol. Chem., 272: 9677-9682, 1997.

Yet another example of an apoptosis assay is the TUNEL assay. Briefly, cell death by apoptosis is characterized by DNA fragmentation in 200-250 and/or 30-50 kilobases. Further internucleosomal DNA fragmentation in 180-200 base pairs may also occur. Such characteristics have been used to distinguish apoptotic cells from normal or necrotic cells. To detect apoptotic cells, whatever the pattern of DNA fragmentation, the TUNEL (Terminal deoxynucleotidyl transferase (TdT) mediated dUTP Nick End Labeling) method is commonly utilized. One of the most easily measured features of apoptotic cells is the break-up of the genomic DNA by cellular nucleases. These DNA fragments can be extracted from apoptotic cells and result in the appearance of “DNA laddering” when the DNA is analyzed by agarose gel electrophoresis. The DNA of non-apoptotic cells, which remains largely intact, does not display this “laddering” on agarose gels during electrophoresis. The large number of DNA fragments appearing in apoptotic cells results in a multitude of 3′-hydroxyl ends in the DNA. This property can be used to identify apoptotic cells by labeling the 3′-hydroxyl ends with bromolated deoxyuridine triphosphate nucleotides (Br-dUTP). The enzyme terminal deoxynucleotidyl transferase (TdT) catalyzes a template independent addition of deoxyribonucleoside triphosphates to the 3′-hydroxyl ends of double- or single-stranded DNA with either blunt, recessed or overhanging ends. See Li and Darzynkiewicz, Cell Prolif., 28: 572-579, 1995.

In another apoptosis assay, the cell death ELISA detects the same endpoint as the TUNEL assay, DNA fragmentation. However, in the cell death ELISA assay, the histone complexed DNA fragments are measured directly by antibodies in an ELISA assay. See Piro, et al., Metabolism, 51:1340-7 (2002); Facchiano et al., Exp. Cell Res., 271: 118-129, 2001; Horigome et al., Immunopharmacology, 37: 87-94, 1997.

### Small Molecule Chemical Composition

“Small molecule” includes any chemical or other moiety that can act to affect biological processes. Small molecules can include any number of therapeutic agents presently known and used, or can be small molecules synthesized in a library of such molecules for the purpose of screening for biological function(s). Small molecules are distinguished from macromolecules by size. The small molecules of this invention usually have molecular weight less than about 5,000 daltons (Da), preferably less than about 2,500 Da, more preferably less than 1,000 Da, most preferably less than about 500 Da.

Small molecules include without limitation organic compounds, peptidomimetics and conjugates thereof. As used herein, the term “organic compound” refers to any carbon-based compound other than macromolecules such nucleic acids and polypeptides. In addition to carbon, organic compounds may contain calcium, chlorine, fluorine, copper, hydrogen, iron, potassium, nitrogen, oxygen, sulfur and other elements. An organic compound may be in an aromatic or aliphatic form. Non-limiting examples of organic compounds include acetones, alcohols, anilines, carbohydrates, monosaccharides, oligosaccharides, polysaccharides, amino acids, nucleosides, nucleotides, lipids, retinoids, steroids, proteoglycans, ketones, aldehydes, saturated, unsaturated and polyunsaturated fats, oils and waxes, alkenes, esters, ethers, thiols, sulfides, cyclic compounds, heterocylcic compounds, imidizoles and phenols. An organic compound as used herein also includes nitrated organic compounds and halogenated (e.g., chlorinated) organic compounds. Methods for preparing peptidomimetics are described below. Collections of small molecules, and small molecules identified according to the invention are characterized by techniques such as accelerator mass spectrometry (AMS; see Turteltaub et al., Curr Pharm Des 6(10): 991-1007, 2000, Bioanalytical applications of accelerator mass spectrometry for pharmaceutical research; and Enjalbal et al, Mass Spectrom Rev 19(3): 139-61, 2000, Mass spectrometry in combinatorial chemistry.)

Preferred small molecules are relatively easier and less expensively manufactured, formulated or otherwise prepared. Preferred small molecules are stable under a variety of storage conditions. Preferred small molecules may be placed in tight association with macromolecules to form molecules that are biologically active and that have improved pharmaceutical properties. Improved pharmaceutical properties include changes in circulation time, distribution, metabolism, modification, excretion, secretion, elimination, and stability that are favorable to the desired biological activity. Improved pharmaceutical properties include changes in the toxicological and efficacy characteristics of the chemical entity.

Intellipeptides interact with tubulin to increase microtubule assembly or inhibit microtubule disassembly. Intellipeptides can also interact with tubulin to decrease thermal aggregation of tubulin.

### Methods of Treatment

Intellipeptides are provided which can be useful in a variety of applications, including, but not limited to, therapeutic uses, e.g., to treat diseases and disorders associated with microtubule assembly and the ability to induce apoptosis in cells in the mammalian subject, including, but not limited to, treatment of neoplastic diseases, cancer, and solid tumors, as well as in the manufacture and purification of polypeptides, including recombinantly-produced polypeptides. It is believed that the ability of a candidate therapeutic compound to increase apoptosis in a cell related to the compound interacting with tubulin to increase microtubule assembly or inhibit microtubule disassembly, or decrease thermal aggregation of tubulin in vitro may be correlated with the ability of the compound to increase apoptosis in vivo. In addition, it is believed that the ability of a candidate therapeutic compound to stabilize the functional structure of a protein in vitro may be correlated with the ability of the compound to assist that protein in performing its function in vivo.

“Pharmaceutically acceptable”, “physiologically tolerable” and grammatical variations thereof, as they refer to compositions, carriers, diluents and reagents, are used interchangeably and represent that the materials are capable of administration to or upon a human without the production of undesirable physiological effects to a degree that would prohibit administration of the composition.

A “therapeutically effective amount” means the amount that, when administered to a subject for treating a disease, is sufficient to effect treatment for that disease.

“Pharmaceutically acceptable excipient” means an excipient that is useful in preparing a pharmaceutical composition that is generally safe, non-toxic, and desirable, and includes excipients that are acceptable for veterinary use as well as for human pharmaceutical use. Such excipients can be solid, liquid, semisolid, or, in the case of an aerosol composition, gaseous.

“Pharmaceutically acceptable salts and esters” means salts and esters that are pharmaceutically acceptable and have the desired pharmacological properties. Such salts include salts that can be formed where acidic protons present in the compounds are capable of reacting with inorganic or organic bases. Suitable inorganic salts include those formed with the alkali metals, e.g. sodium and potassium, magnesium, calcium, and aluminum. Suitable organic salts include those formed with organic bases such as the amine bases, e.g. ethanolamine, diethanolamine, triethanolamine, tromethamine, N methylglucamine, and the like. Such salts also include acid addition salts formed with inorganic acids (e.g., hydrochloric and hydrobromic acids) and organic acids (e.g., acetic acid, citric acid, maleic acid, and the alkane- and arene-sulfonic acids such as methanesulfonic acid and benzenesulfonic acid). Pharmaceutically acceptable esters include esters formed from carboxy, sulfonyloxy, and phosphonoxy groups present in the compounds, e.g. C₁₋₆ alkyl esters. When there are two acidic groups present, a pharmaceutically acceptable salt or ester can be a mono-acid-mono-salt or ester or a di-salt or ester; and similarly where there are more than two acidic groups present, some or all of such groups can be salified or esterified. Compounds named in this invention can be present in unsalified or unesterified form, or in salified and/or esterified form, and the naming of such compounds is intended to include both the original (unsalified and unesterified) compound and its pharmaceutically acceptable salts and esters. Also, certain compounds named in this invention can be present in more than one stereoisomeric form, and the naming of such compounds is intended to include all single stereoisomers and all mixtures (whether racemic or otherwise) of such stereoisomers.

Except when noted, “subject” or “patient” are used interchangeably and refer to mammals such as human patients and non-human primates, as well as experimental animals such as rabbits, rats, and mice, and other animals. Accordingly, the term “subject” or “patient” as used herein means any mammalian patient or subject to which the compositions can be administered. In some embodiments of the present invention, the patient will be suffering from neoplastic disease. In an exemplary embodiment of the present invention, to identify subject patients for treatment with a pharmaceutical composition comprising peptides, peptide analogs or peptide mimetics according to the methods, accepted screening methods are employed to determine the status of an existing disease or condition in a subject or risk factors associated with a targeted or suspected disease or condition. These screening methods include, for example, examinations to determine whether a subject is suffering from an disease. These and other routine methods allow the clinician to select subjects in need of therapy.

The peptides presented here provide a versatile set of drug molecules that can be customized for use as therapeutic peptides to treat or prevent neoplastic diseases, cancer, and solid tumors. Examples of diseases related to neoplastic disease include, but are not limited to, any of a number of diseases that are characterized by uncontrolled, abnormal proliferation of cells, the ability of affected cells to spread locally or through the bloodstream and lymphatic system to other parts of the body (i.e., metastasize) as well as any of a number of characteristic structural and/or molecular features. A “cancerous” or “malignant cell” or “solid tumor cell” is understood as a cell having specific structural properties, lacking differentiation and being capable of invasion and metastasis. “Neoplastic disease” or “cancer” refers to all types of cancer or neoplasm or malignant tumors found in mammals, including carcinomas, sarcomas, lymphomas and leukemias. Examples are cancers of the breast, lung, stomach, and oesophagus, brain and nervous system, head and neck, bone, liver, gall bladder, pancreas, colon, genitourinary system, urinary bladder, urinary system, kidney, testes, uterus, ovary, prostate, skin and skin appendices, melanoma, mesothelioma, endocrine system.

Accordingly, the present invention describes a variety of methods related to the use of Intellipeptides. In one embodiment, the present invention provides a method of inducing apoptosis in a cell wherein the Intellipeptides interacts with tubulin to promote microtubule assembly, inhibit microtubule disassembly, or decrease thermal aggregation of tubulin, by providing an Intellipeptide to a cell or solution comprising said protein. In a related embodiment, the present invention includes a method for treating neoplastic diseases, cancer, and solid tumors, by providing an Intellipeptide to a cell or solution comprising said protein.

Intellipeptides may be provided to a cell or solution by a variety of means available in the art. For example, synthesized Intellipeptides may be directly provided to a solution or into a cell. In addition, Intellipeptides may be provided to a cell or solution by introducing an expression vector comprising a polynucleotide sequence encoding an Intellipeptide with regulatory elements that drive expression of said Intellipeptide in a cell. The polynucleotide sequence may further comprise additional coding regions, including, e.g., a secretion signal such that the Intellipeptide will be secreted from the cell and/or additional elements regulating expression of the encoded Intellipeptide, of which a large variety are known and available in the art, including those used for inducible expression of peptides and polypeptides. Thus, the present invention further includes polynucleotide sequences encoding Intellipeptides and expression vectors comprising the same, including, e.g., viral vectors.

Intellipeptides can be used as therapeutics for, but not limited to, the treatment of diseases and disorders associated with microtubule assembly and the ability to induce apoptosis in cells in the mammalian subject, e.g., in methods for treatment of neoplastic diseases, cancer, and solid tumors in a mammalian subject. Intellipeptides are a versatile set of molecules that interact with tubulin to promote microtubule assembly, inhibit microtubule disassembly, or decrease thermal aggregation of tubulin, and therein induce apoptosis in cells in a mammalian subject include, but are not limited to, treatment of neoplastic diseases, cancer, and solid tumors.

### EXEMPLARY EMBODIMENTS

### Example 1

### Effect of Synthetic Peptides on Microtubule Assembly and Disassembly and Tubulin Aggregation

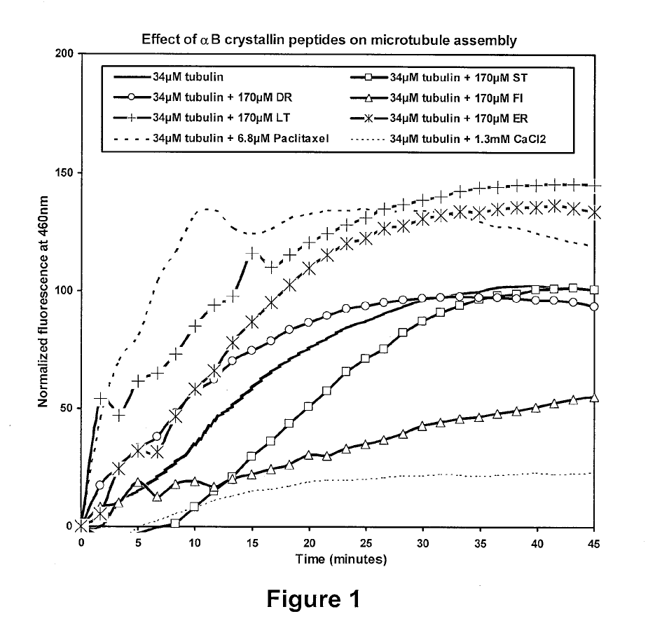

The effect of synthetic peptides corresponding to five interactive sequences of αB crystallin on microtubule assembly and disassembly and tubulin aggregation was investigated (FIG. 1). When 34 μM tubulin alone was incubated at 37° C., a rapid increase in DAPI fluorescence was observed due to the preferential binding of DAPI to assembled microtubules and maximum fluorescence was observed in approximately 45 minutes. The ST peptide slowed the rate of microtubule assembly by increasing the lag phase preceding the start of microtubule assembly but had no effect on the amount of microtubules formed in 45 minutes. The DR peptide accelerated microtubule assembly but had no effect on the total amount of microtubules formed in 45 minutes. In contrast, the FI peptide slowed microtubule assembly and decreased the amount of microtubules formed in 45 minutes. The LT and ER peptides increased both the rate of microtubule assembly and the amount of microtubules formed in 45 minutes. The effect of the LT and ER peptides was similar to Paclitaxel, a known promoter of microtubule assembly, while the effect of the FI peptide was similar but weaker than the effect of CaCl₂, a known inhibitor of microtubule assembly.

FIG. 1 shows an effect of αB crystallin peptides on microtubule assembly. The effects of five synthetic αB crystallin peptides ₄₁STSLSPFYLRPPSFLRAP₅₈ (ST), ₇₃DRFSVNLDVKHFS₈₅ (DR), ₁₁₃FISREFHR₁₂₀ (FI), ₁₃₁LTITSSLSSDGV₁₄₂ (LT), and ₁₅₆ERTIPITRE₁₆₄ (ER) on microtubule assembly were studied using a fluorescence assay. Bonne et al., J Biol Chem 260:2819-25, 1985. Samples containing tubulin and αB crystallin peptides or control molecules were excited at λ=355 nm and the fluorescence emission of DAPI bound to assembled microtubules was measured at λ=460 nm. The fluorescence of the sample containing tubulin alone increased rapidly to a maximum value at 45 minutes of incubation at 37° C. In the presence of the ST peptide, the initiation of microtubule assembly began approximately 5 minutes later than tubulin alone and at 45 minutes, no change in total microtubule assembly was observed. In the presence of the DR peptide, the rate of assembly increased without an effect on total microtubule assembly after 45 minutes. In the presence of the FI peptide, microtubule assembly was inhibited. In the presence of the LT and ER peptides, both the rate of assembly and total microtubule assembly increased. The positive control, Paclitaxel accelerated microtubule assembly, while the negative control CaCl₂ inhibited microtubule assembly which was consistent with previous reports. Thompson et al., Cell Motil 1:445-54, 1981; Berkowitz, S. A. and Wolff, J., J Biol Chem 256:11216-23, 1981. The FI peptide inhibited microtubule assembly while the LT and ER peptides promoted microtubule assembly.

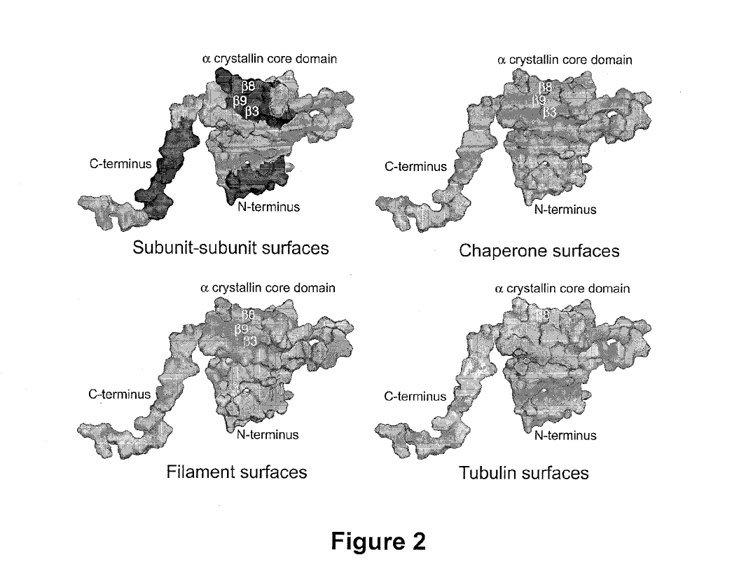

αB crystallin sequences that altered microtubule assembly overlapped with sequences previously identified as interactive sequences for subunit-subunit interactions chaperone activity, and filament interactions in αB crystallin, (Ghosh, J. G. and Clark, J. I., Protein Sci 14:684-95, 2005; Ghosh, et al., Biochemistry 44:14854-69, 2005) (FIG. 2). The FI peptide overlapped with sequences for chaperone activity and filament interactions. while the LT and ER peptides overlapped with sequences for subunit-subunit interactions, chaperone activity, and filament interactions. The overlap between the αB crystallin sequences that altered microtubule assembly and the αB crystallin sequences used for chaperone activity suggested a functional role for αB crystallin in tubulin/microtubule stabilization.

FIG. 2 shows surface locations of the interactive sequences in αB crystallin for subunit-subunit interactions, chaperone activity, and interactions with filaments and tubulin. Interactive sequences for subunit-subunit interactions, chaperone activity and interactions with filaments and tubulin identified by in vitro assays, mutagenesis, and pin array analysis were mapped to the β3-β8-β9 interface region and the N- and C-terminal extensions in the 3D homology model for human αB crystallin described previously. Ghosh, J. G. and Clark, J. I., Protein Sci 14:684-95, 2005; Ghosh, et al., Biochemistry 44:14854-69, 2005; Ghosh et al., Cell Stress Chaperones 11:187-97; Ghosh et al., Biochemistry 45:13847-13854, 2006. Surfaces formed by the LT (β8) and ER (C-terminus extension containing the Ile-X-Ile motif) sequences mediated subunit-subunit interactions as well as interactions with unfolded substrate proteins, filaments, and tubulin.

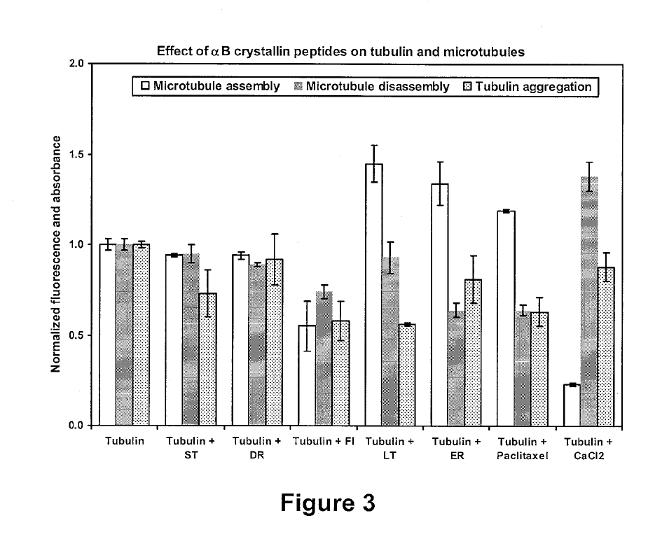

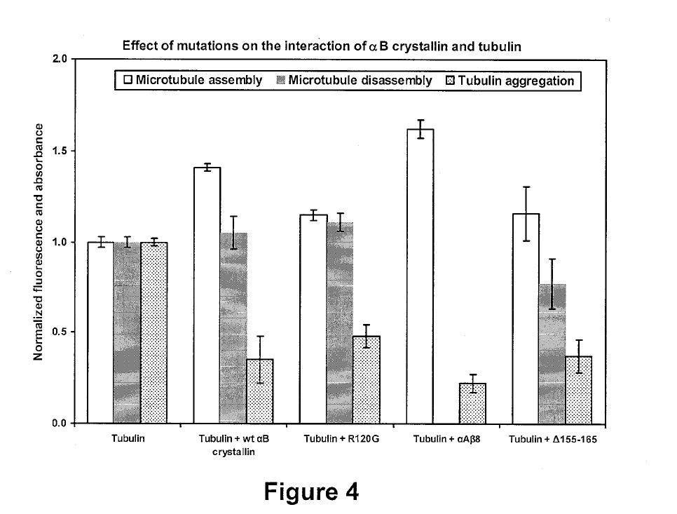

αB crystallin interactive sequences that protected microtubules against destabilization and disassembly were identified by measuring the effect of the five αB crystallin interactive sequences on the disassembly of microtubules (FIG. 3). Pre-formed microtubules (34 μM) were incubated in the absence and presence of αB crystallin peptides and controls at 23° C. to induce disassembly of microtubules. In the absence of αB crystallin peptides and controls, microtubules alone disassembled rapidly and minimum fluorescence was recorded in approximately 20 minutes. The FI and ER peptides inhibited microtubule disassembly by ˜24% and 36% respectively similar to the microtubule-stabilizing molecule Paclitaxel, while the remaining peptides conferred little to no protection against the disassembly of microtubules.

The interactive sequences in αB crystallin that protected tubulin from unfolding and aggregation were identified by measuring the effect of the αB crystallin peptides on the thermal aggregation of tubulin (FIG. 3). The ability of the αB crystallin peptides to protect against the thermal aggregation of tubulin was determined by measuring the optical density (OD₃₄₀) of 34 μM tubulin at 52° C. for sixty minutes in the absence or presence of peptides and control molecules. In the absence of αB crystallin peptides and controls, tubulin aggregated rapidly and a maximum optical density was recorded in approximately 60 minutes. The α crystallin core domain peptides FI and LT had the strongest protective effects and decreased the aggregation of tubulin by ˜42-44%. In contrast, the N-terminal peptide ST, the core domain peptide DR, and the C-terminal peptide, ER, had weak protective effects and the aggregation of tubulin incubated with these peptides decreased by only 8-27% relative to the control. Microtubule assembly/disassembly and thermal aggregation assays identified the ST, FI, LT, and ER peptides as interactive sequences in αB crystallin that were important for the dynamic assembly of microtubules.

FIG. 3 shows an effect of synthetic αB crystallin peptides on microtubule assembly, disassembly, and tubulin aggregation. The DAPI fluorescence of assembled microtubules, disassembled tubulin, and tubulin aggregates in the absence of αB crystallin peptides and control additives were normalized to 1.0. In the presence of the ST peptide, no effect on microtubule assembly and disassembly was observed, and a small protective effect against tubulin aggregation was observed. In the presence of the DR peptide, no effect on microtubule assembly, disassembly, and tubulin aggregation was observed. In the presence of the FI peptide, microtubule assembly, disassembly, and tubulin aggregation decreased. In contrast, in the presence of the LT peptide, microtubule assembly increased, tubulin aggregation decreased, and no effect on microtubule disassembly was observed. In the presence of the ER peptide, microtubule assembly increased, and microtubule disassembly and tubulin aggregation decreased. The FI, LT, and ER peptides had the strongest effect on microtubule assembly/disassembly and tubulin aggregation, while ST and DR peptides had little to no effect microtubule assembly/disassembly and tubulin aggregation.

Microtubule assembly and disassembly, and tubulin aggregation assays were conducted with αB crystallin mutants R120G, αAβ8, and Δ155-165, which contained mutations at sites corresponding to the FI, LT, and ER peptides respectively to confirm the results obtained with the synthetic peptides (FIG. 4). Wt αB crystallin increased microtubule assembly by ˜41%, had no effect on the microtubule disassembly, and decreased the thermal aggregation of tubulin by 65%. With the αB crystallin mutant R120G, which contains a single point mutation in the ₁₁₃FISREFHR₁₂₀ sequence, microtubule assembly and disassembly were unchanged while tubulin aggregation decreased. The αB crystallin mutant αAβ8, which contains multiple mutations at residues corresponding to the ₁₃₁LTITSSLS₁₃₈ sequence increased microtubule assembly, completely inhibited microtubule disassembly, and decreased tubulin aggregation. The Δ155-165 mutant, which lacks residues 155-165 corresponding to the ER peptide, increased microtubule assembly, and decreased both microtubule disassembly and tubulin aggregation. The results confirmed the importance of the αB crystallin sequences ₁₁₃FISREFHR₁₂₀, ₁₃₁LTITSSLSSDGV₁₄₂, and ₁₅₆ERTIPITRE₁₆₄ in microtubule assembly, disassembly and aggregation.

FIG. 4 shows an effect of mutations in three αB crystallin interactive domains on microtubule assembly, disassembly, and tubulin aggregation. The DAPI fluorescence of assembled microtubules, disassembled tubulin, and tubulin aggregates in the absence of αB crystallin mutants was normalized to 1.0. In the presence of wt αB crystallin, microtubule assembly increased, microtubule disassembly was unchanged, and tubulin aggregation decreased. In the presence of the R120G mutant, which contains a mutation of the Arg-120 residue in the ₁₁₃FISREFHR₁₂₀ interactive sequence of αB crystallin, microtubule assembly was lower and microtubule disassembly and tubulin aggregation were similar to wt αB crystalline. In the presences of the αAβ8 mutant, in which the β8 sequence ₁₃₁LTITSSLS₁₃₈ of αB crystallin was replaced with the β8 sequence of αA crystallin ₁₂₇SALSCLSS₁₃₄, microtubule assembly increased, microtubule disassembly decreased, and tubulin aggregation was unchanged relative to wt αB crystalline. In the presence of the C-terminal deletion mutant Δ155-165, microtubule assembly and disassembly were lower and tubulin aggregation was unchanged relative to wt αB crystalline Mutagenesis of sequences in αB crystallin corresponding to the αB crystallin peptides that altered tubulin-microtubule dynamics confirmed the effects of the αB crystallin peptides on microtubule assembly/disassembly and tubulin aggregation.

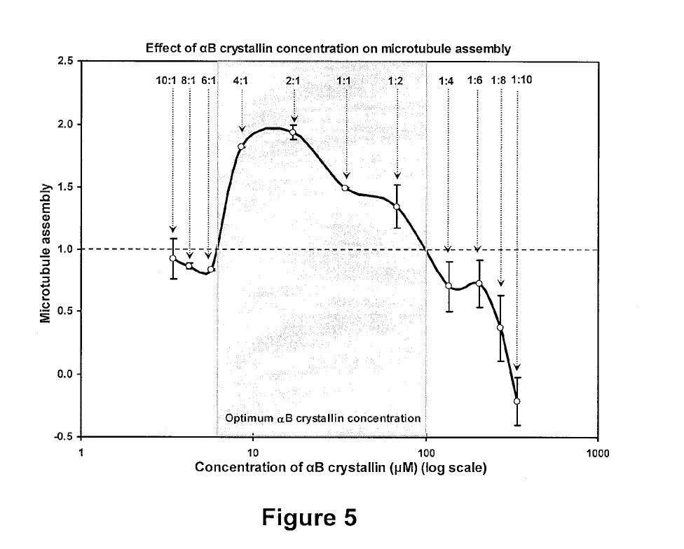

To evaluate the concentration dependence of αB crystallin on the assembly and disassembly of microtubules, a fixed amount (34 μM) of tubulin was incubated with increasing concentrations of wt αB crystallin (FIG. 5). At low concentrations of wt αB crystallin, no measurable effect on microtubule assembly was observed. With increasing concentrations of αB crystallin, microtubule assembly increased to a maximum and then declined at high concentrations where microtubule assembly was inhibited. With respect to the ratio of tubulin to αB crystallin, the effect on assembly of microtubules was minimal when the ratio of tubulin to αB crystallin was >4:1. When the ratio of tubulin to αB crystallin was between 4:1 and 1:2, the amount of microtubules formed was 35-94% higher than tubulin alone and microtubule assembly was optimal for a ratio of tubulin to αB crystallin of 2:1. When the ratio of tubulin to αB crystallin was <1:2 the amount of microtubules formed was 30-63% less than for tubulin alone and no microtubules were formed when the ratio of tubulin to αB crystallin was 1:10. Wt αB crystallin stabilized microtubules in a concentration dependent manner and was most effective within a narrow concentration range.

FIG. 5 shows an effect of αB crystallin concentration on microtubule assembly. Microtubule assembly (Y-axis) was sensitive to the concentration of wt αB crystallin (X-axis). Microtubule assembly in the absence of αB crystallin was normalized to 1.0. The ratio of tubulin to αB crystallin for each concentration of αB crystallin is listed at the top of the plot. For tubulin to αB crystallin ratios >4:1, microtubule assembly was unchanged at 1.0. For ratios between 4:1 and 1.2, microtubule assembly was >1.0 with maximum assembly observed at a tubulin to αB crystallin ratio of 2:1. For ratios <1.4, microtubule assembly was <1. For a tubulin to αB crystallin ratio of 1:10, microtubule assembly was undetectable. The variation of microtubule assembly with the concentration of human αB crystallin may be explained on the basis of the dynamic quaternary structure of αB crystalline.

### Example 2

### αB Crystallin Sequences Interact with Tubulin to Promote Microtubule Assembly

Five interactive sequences in the sHSP and molecular chaperone, human αB crystallin participated in the assembly of tubulin to microtubules. Synthetic αB crystallin peptides and mutants either promoted or inhibited microtubule assembly and disassembly. Synthetic peptides corresponding to the αB crystallin sequences ₁₃₁LTITSSLSSDGV₁₄₂ and ₁₅₅ERTIPITRE₁₆₅ interacted with tubulin to promote microtubule assembly. In contrast, the synthetic peptide corresponding to the ₁₁₃FISREFHR₁₂₀ sequence inhibited microtubule assembly. The remaining αB crystallin sequences ₄₁STSLSPFYLRPPSFLRAP₅₈ and ₇₃DRFSVNLDVKHFS₈₅ had no effect on microtubule assembly. The results were consistent with previous reports in which full-length αB crystallin interacted with tubulin and modulated the assembly of tubulin into microtubules. Fujita et al., J Cell Sci 117:1719-26, 2004; Atomi et al., Biol Sci Space 15:206-7, 2001. In thermal aggregation assays, the interactive sequences ₁₁₃FISREFHR₁₂₀ and ₁₃₁LTITSSLSSDGV₁₄₂ protected disassembled tubulin from unfolding and aggregation. The results were consistent with previous studies reporting that αB crystallin protects tubulin from unfolding and aggregation under stress. Sakurai et al., Faseb J, 2005; Xi et al., Faseb J 20:846-57, 2006; Day et al, Cell Stress Chaperones 8:183-93, 2003; Arai, H. and Atomi, Y., Cell Struct Funct 22:539-44, 1997. Individual synthetic αB crystallin peptides provided modest protection against tubulin aggregation (<44% protection) and full-length αB crystallin had a strong protective effect (˜65% protection) on the thermal aggregation of tubulin, suggesting that the 3D organization of αB crystallin was important for the collective activity of the αB crystallin interactive sequences.

In previous studies, we applied protein pin array technology, site-directed mutagenesis, and size exclusion chromatography to demonstrate the importance of the N-terminal sequence ₄₁STSLSPFYLRPPSFLRAP₅₈, the α crystallin core domain sequences, ₇₃DRFSVNLDVKHFS₈₅, ₁₁₃FISREFHR₁₂₀, and ₁₃₁LTITSSLSSDGV₁₄₂, and a sequence in the C-terminal extension, ₁₅₆ERTIPITRE₁₆₄, in subunit-subunit interactions, chaperone activity, and filament interactions. Ghosh, J. G. and Clark, J. I., Protein Sci 14:684-95, 2005; Ghosh, et al., Biochemistry 44:14854-69,200; Ghosh et al., Cell Stress Chaperones 11:187-97; Ghosh et al., Biochemistry 45:13847-13854, 2006. 3D computer modeling mapped these sequences to interactive surfaces in the N-terminus, a crystallin core domain, and the C-terminal extension of the human αB crystallin monomer (Ghosh, J. G. and Clark, J. I., Protein Sci 14:684-95, 2005; Ghosh, et al., Biochemistry 44:14854-69, 200; Ghosh et al., Cell Stress Chaperones 11:187-97; Ghosh et al., Biochemistry 45:13847-13854, 2006) (FIG. 2). The presence of overlapping interactive sequences that mediated interactions with multiple proteins suggested that the chaperone activity of αB crystallin was independent of the size of the αB crystallin complex but required surface exposure of interactive sequences for interactions with unfolding target proteins 29; 30; 31; 32; 33. Ghosh, J. G. and Clark, J. I., Protein Sci 14:684-95, 2005; Ghosh, et al., Biochemistry 44:14854-69, 200; Ghosh et al., Cell Stress Chaperones 11: 187-97; Liu et al., Anal Biochem 350:186-95, 2006; Ghosh et al., Biochemistry 45:13847-13854, 2006. The model is consistent with a dynamic subunit model for αB crystallin function in cells in which the dissociation of αB crystallin subunits from a crystallin complexes and/or filament networks regulates association with unfolded substrate proteins, and re-association into a crystallin-substrate complexes. Liu et al., Anal Biochem 350:186-95, 2006. The relative affinity of αB crystallin for itself and its selected substrates may explain the significance of the dynamic subunit model for sHSP complex assembly in regulation of sHSP structure and function. Liu et al., Anal Biochem 350:186-95, 2006.

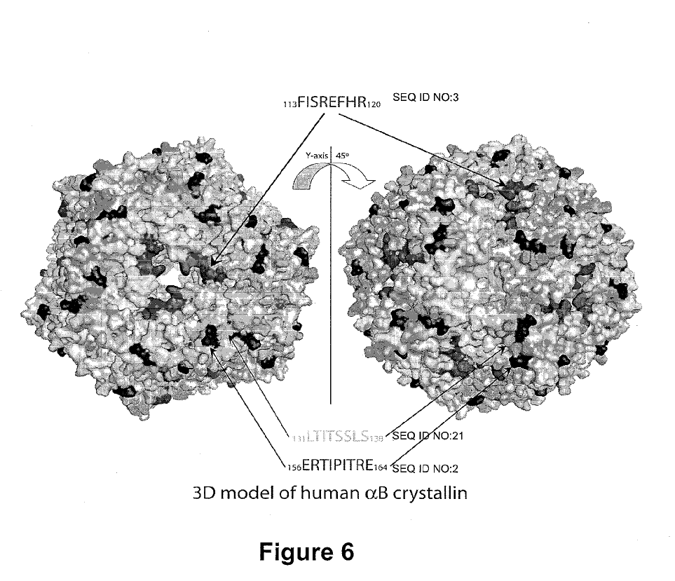

The relationship between microtubule assembly and the concentration of αB crystallin is consistent with the dynamic model for the assembly of αB crystallin complexes 32. The LT and ER sequences that promote microtubule assembly were previously demonstrated to be critical for the normal dynamic assembly and disassembly of multimeric αB crystallin complexes (Ghosh, J. G. and Clark, J. I., Protein Sci 14:684-95, 2005; Liu et al., Anal Biochem 350:186-95, 2006; Kim et al., Nature 394:595-9, 1998; van Montfort et al., Adv Protein Chem 59:105-56, 2001; vanMontfort et al., Nat Struc Biol 8:1025-30, 2001; Pasta et al., Mol V is 10:655-62, 2004; Liang, J. J. and Liu, B. F., Protein Sci 15:1619-27, 2006; Studer, S. and Narberhaus, F., J Biol Chem 275:37212-8, 2000) (FIGS. 2 and 6). At high αB crystallin concentrations and small tubulin:αB crystallin ratios (<1:4), where it is expected that the assembled complex was the predominant form of αB crystallin, microtubule assembly was inhibited. Under these conditions, the LT and ER sequences in apposed αB crystallin subunits interacted with each other and were unavailable to interact with tubulin/microtubules because they were partially buried in the complex (FIG. 6). In contrast, the FI sequence remained accessible on the surface of the complex, interacted with tubulin and inhibited microtubule assembly (FIG. 6). At low αB crystallin concentrations and large tubulin:αB crystallin ratios (>4:1), the amount of αB crystallin present is insufficient to modulate microtubule assembly and there was little or no effect on microtubule assembly. At intermediate concentrations of αB crystallin and tubulin:αB crystallin ratios between 4:1 and 1:2, αB crystallin stabilized microtubules and favored the assembly of additional microtubules. Under these conditions, the LT and ER sequences were exposed on the surface of dissociated αB crystallin subunits, interacted with tubulin, and promoted microtubule assembly. The results demonstrated that interactive sequences on the surface of αB crystallin may interact co-operatively and competitively with tubulin to stabilize microtubules and modulate the dynamic assembly of microtubules. The overlap between interactive sites for assembly, chaperone activity, and filament interactions and their 3D organization on the surface of αB crystallin subunits (FIG. 2) supports the dynamic subunit model for the physiological function of αB crystallin, which involves the dynamic association, dissociation, and re-association of αB crystallin with itself and its substrates. If this interpretation is correct, measurement of the relative affinities between αB crystallin subunits will confirm the hypothesis that dynamic subunit assembly is responsible for the observed relationship between microtubule assembly and αB crystallin concentration. Quantitative studies are being conducted using surface plasmon resonance (SPR) to test this hypothesis.

FIG. 6 shows a model of the tubulin interactive sequences in the human αB crystallin complex and their importance in the assembly of microtubules. In the model, twenty-four subunits (grey) of αB crystallin form a complex which is a hollow sphere containing eight windows entering the central cavity. Kim et al., Nature 394:595-9, 1998; van Montfort et al., Adv Protein Chem 59:105-56, 2001; vanMontfort et al., Nat Struc Biol 8:1025-30, 2001; Haley et al., J Mol Biol 277:27-35, 1998. The αB crystallin sequences that modulate tubulin-microtubule dynamics are in red (₁₁₃FISREFHR₁₂₀), green (₁₃₁LTITSSLS₁₃₈), and blue (₁₅₆ERTIPITRE₁₆₄). The FISREFHR sequence, which inhibits microtubule assembly, is exposed on the surface of the hollow αB crystallin complex. FISREFHR sequences from three separate αB crystallin subunits surround each of the eight windows that lead into the hollow core of the complex. In contrast, the ₁₃₁LTITSSLS₁₃₈ and ₁₅₆ERTIPITRE₁₆₄ sequences, which promote microtubule assembly, are sites of subunit-subunit interactions in αB crystallin with limited exposure on the surface of the complex. For these sequences to interact with tubulin and promote microtubule assembly, dissociation of the subunits from the complex is required. In contrast, tubulin binding to the inhibitory FISREFHR sequences can occur on the surface of the complex. The computed model for the human αB crystallin complex was based on the Methanococcus jannaschii sHSP16.5 24-subunit crystal structure described previously. Muchowski et al., J Mol Bio 1289:397-411, 1999.

The results confirm the importance of sHSPs in the assembly of microtubules and their possible role in amyloid cascade pathway (formation of amyloid fibrils→hyperphosphorylation of tau→disruption of tau-tubulin interactions→formation of neurofibrillary tangles (NFTs)→neurodegeneration). Although various studies support the amyloid cascade hypothesis, the constitutive expression of sHSPs in the brain is low and sHSPs including αB crystallin are major constituents of amyloid plaques in Alzheimer's disease patients. Wilhelmus et al., Neuropathl Appl Neurobiol 32:119-30, 2006; Renkawek et al., Acta Neuropathol (Berl) 87:155-60, 1994; Shinohara et al., J Neurol Sci 119:203-8, 1993. High concentrations of the sHSP and molecular chaperone αB crystallin promote microtubule disassembly, which suggests that the in vivo over-expression and extracellular secretion of αB crystallin in response to amyloid-β could trigger the destabilization of tau-tubulin bundles and lead to the formation of neurofibrillary tangles. This hypothesis is consistent with the observation that αB crystallin associates with extracellular NFTs (Mao et al., Neuropathol Appl Neurobiol 27:180-8, 20010 but not intracellular NFTs. Wilhelmus et al., Neuropathl Appl Neurobiol 32:119-30, 2006. Microtubule stabilizers may have therapeutic value in neurodegenerative diseases such as Alzheimer's disease where hyper-phosphorylation of the microtubule associated protein tau results in the disintegration of microtubules and the formation of NFTs. Attard et al., Pathol Biol (Paris) 54:72-84, 2006; Jordan et al., Med Res Rev 18:259-96, 1998. The expression of sHSPs including αB crystallin in the progression of Alzheimer's disease needs further study to determine the protective function of sHSPs in neurodegeneration.

The results in this study may have therapeutic significance in the identification of novel sequences in sHSPs as anti-cancer agents. Gruvberger-Saal, S. K. and Parsons, R. J Clin Invest 116:30-2, 2006; Laudanski, K. and Wyczechowska, D., Arch Immunol Ther Exp (Warz), 2006. Peptides that interact with microtubules to prevent their disassembly can interrupt mitosis, preventing cell division, and triggering apoptosis. Modulation of microtubule assembly is of great interest in the development of new cancer treatments. Attard et al., Pathol Biol (Paris) 54:72-84, 2006; Schiff, P. B. and Horwitz, S. B., Proc Natl Acad Sci USA 77:1561-5, 1980; Montero et al., Lancet Oncol 6:229-39, 2005; Clavarezza et al., Ann Oncol 17 Suppl 7:vii22-vii26, 2006; Simmons et al., Mol Cancer Ther 4:333-42, 2005; Bai et al., Biochem Pharmacol 39:1941-9, 1990. Two of the most important anti-cancer drugs today, Paclitaxel and Docetaxel are examples of molecules that stabilize microtubules and prevent their disassembly. However, undesirable side effects including drug resistance limit the effectiveness of many current anti-cancer agents. The αB crystallin peptides LTITSSLSSDGV and ERTIPITRE that disrupt tubulin [symbol] microtubule dynamics can be developed into safe new therapeutics for cancer, Alzheimer's disease, and taupathies.

In summary, interactive sequences on the surface of αB crystallin selectively recognize tubulin/microtubules to regulate assembly and stabilization of microtubules and/or protect against the destabilization and disassembly of microtubules depending on the relative concentration of tubulin to αB crystallin subunits.

### Example 3

### Materials and Methods

Materials. Synthetic αB crystallin peptides DRFSVNLDVKHFS (DR), STSLSPFYLRPPSFLRAP (ST), FISREFHR (FT), LTITSSLSSDGV (LT), and ERTIPITRE (ER) were procured from Advanced ChemTech (Louisville, Ky.) and Genscript Corporation (Piscataway, N.J.).

Construction, expression, and purification of wt and mutant αB crystallins. The αB crystallin mutants were constructed using the Quick-Change site-directed mutagenesis kit as described previously. Ghosh et al., Cell Stress Chaperones 11:187-97; Ghosh et al., Biochemistry 45:13847-13854, 2006; Perng et al., J Biol Chem 274:33235-43, 1999; Ghosh et al., Biochemistry 45:9878-86, 2006. The R120G mutant is a single point mutant of the ₁₁₃FISREFHR₁₂₀ sequence of human αB crystallin, constructed by replacing Arg-120 with a glycine residue. The αAβ8 mutant was constructed by replacing the α crystallin core domain β8 sequence ₁₃₁LTITSSLS₁₃₈ of human αB crystallin with the homologous β8 sequence ₁₂₇SALSCSLS₁₃₄ of human αA crystalline. The Δ155-165 mutant was constructed by deleting residues ₁₅₅ERTIPITRE₁₆₅ from the C-terminus extension of human αB crystalline Wt αB crystallin, R120G, αAβ8, and Δ155-165 were expressed and purified as described previously. Ghosh et al., Biochemistry 45:13847-13854, 2006; Ghosh et al., Biochemistry 45:9878-86, 2006.

Microtubule assembly assays. The effect of selected αB crystallin peptides on the in vitro assembly of tubulin into microtubules was evaluated using the Microtubule Stabilization/Destabilization Assay kit (Cytoskeleton; Denver, Colo.) as described previously. Bonne et al., J Biol Chem 260:2819-25, 1985. Bovine brain tubulin was dissolved to 200 μM in 80 mM PIPES, 2 mM MgCl₂, 0.5 mM EGTA, 10 μM DAPI, 1 mM GTP pH 6.9. 8.5 μl of the tubulin was mixed with 40 μl of 80 mM PIPES, 2 mM MgCl₂, 0.5 mM EGTA, 7.4 μM DAPI, 16% Glycerol, 1.1 mM GTP pH 6.9 and 4.3 μl of 2 mM peptide in 2.5% DMSO, 2 mM Paclitaxel (polymerization promoter) in 100% DMSO, 15 mM CaCl₂ (polymerization inhibitor) in water, or 2.5% DMSO only. Microtubule assembly was monitored by measuring the fluorescence of DAPI, a molecule whose emission fluorescence at λ=460 is enhanced 8-fold when it is incorporated into assembled microtubules. Bonne et al., J Biol Chem 260:2819-25, 1985. Fluorescence of samples were continuously read on a Perkin Elmer Victor³ V fluorescence plate reader (Excitation λ=355 nm, Emission λ=460 nm) at 37° C. for 45 minutes.

The effect of wt and three mutant αB crystallins, Δ41-58, αAβ8, and Δ155-165 on the in vitro assembly of tubulin into microtubules was evaluated using the Microtubule Stabilization/Destabilization Assay kit described above (Cytoskeleton; Denver, Colo.). Bovine brain tubulin was dissolved to 200 μM in 80 mM PIPES, 2 mM MgCl₂, 0.5 mM EGTA, 10 μM DAPI, 1 mM GTP pH 6.9. 8.5 μl of the tubulin was mixed with 40 μl of 80 mM PIPES, 2 mM MgCl₂, 0.5 mM EGTA, 7.4 μM DAPI, 16% Glycerol, 1.1 mM GTP pH 6.9 and 4.3 μl of 80 μM protein in 20 mM Tris-Cl, pH8.0 or Tris-Cl buffer only. Fluorescence of samples were continuously read on a Perkin Elmer Victor³ V fluorescence plate reader (Excitation λ=355 nm, Emission λ=460 nm) at 37° C. for 45 minutes.

Microtubule disassembly assays. The effect of αB crystallin peptides and mutants on the in vitro disassembly of microtubules into soluble tubulin was evaluated using the Microtubule Stabilization/Destabilization Assay kit described above (Cytoskeleton; Denver, Colo.) as described previously³⁶. 34 μM pre-formed microtubules were incubated with the αB crystallin peptides (170 μM) or wt and mutant αB crystallins (6.8 μM and 34 μM) at 23° C. for 20 minutes. Incubation of microtubules at 23° C. results in the spontaneous disassembly of microtubules. The decrease in DAPI fluorescence at λ=460 nm was measured continuously for 20 minutes by exciting the samples at λ=355 nm using a Perkin Elmer Victor³ V fluorescence plate reader.

Tubulin aggregation assays. The effect of αB crystallin peptides and mutants on the thermal aggregation of tubulin was evaluated using ultra-violet spectroscopy. Bovine brain tubulin was dissolved to 200 μM in 80 mM PIPES, 2 mM MgCl₂, 0.5 mM EGTA, pH 6.9. 4.25 μl of 0.08, 0.4, or 2 mM test peptide or protein was diluted into 40 μl of 80 mM PIPES, 2 mM MgCl₂, 0.5 mM EGTA, pH 6.9. 8.5 μl of the 200 μM tubulin was added to each sample. Samples were heated at 52° C. and the absorbance at λ=340 nm was measured continuously for 60 minutes using a Pharmacia Biotech Ultrospec 3000. GTP and glycerol were not present in the samples because they induce the assembly of microtubules.

This description of the invention, will enable those skilled in the art to perform within a wide range of equivalent parameters, concentrations, and conditions without departing from the spirit and scope of the invention and without due experimentation results that are presented here.

While this invention has been described in connection with specific embodiments thereof, it will be understood that it is capable of further modifications. This application is intended to cover any variations, uses, or adaptations of the inventions following, in general, the principles of the invention and including such departures from the present disclosure as come within known or customary practice within the art to which the invention pertains and as may be applied to the essential features hereinbefore set forth as follows in the scope of the appended claims.

All references cited herein, including journal articles or abstracts, published or corresponding U.S. or foreign patent applications, issued U.S. or foreign patents, or any other references, are entirely incorporated by reference herein, including all data, tables, figures, and text presented in the cited references. Additionally, the entire contents of the references cited within the references cited herein are also entirely incorporated by reference.

1. A method for treating a neoplastic disease in a mammalian subject comprising administering a polypeptide to the subject in need thereof, wherein the polypeptide is X₁-LTITSSLSSDGV-X₂ (SEQ ID NO:4), or a functional variant or mimetic thereof, wherein each X₁ and X₂ independently of one another represents any amino acid sequence of n amino acids, n varying from 0 to 50, and n being identical or different in X₁ and X₂, in an amount effective to reduce or eliminate the neoplastic disease in the subject.

2. The method of claim 1 wherein the functional variant or mimetic comprises a conservative amino acid substitution or peptide mimetic substitution.

3. The method of claim 1 wherein the functional variant comprises about 70% or greater amino acid sequence identity to X₁-LTITSSLSSDGV-X₂ (SEQ ID NO:4.

4. The method of claim 1 wherein the functional variant has about 70% or greater amino acid sequence identity to X₁-LTITSSLS-X₂ (SEQ ID NO:5).

5. The method of claim 4 wherein the functional variant is X₁-LTITSSLS-X₂ (SEQ ID NO:5).

6. The method of claim 1 wherein the neoplastic disease is a solid tumor, carcinoma, sarcoma, lymphoma, or leukemia.

7. The method of claim 1 wherein the functional variant is a D-enantiomer of one or more amino acids.

8. The method of claim 1 wherein the functional variant is an L-enantiomer of one or more amino acids.

9. A method for treating a neoplastic disease in a mammalian subject comprising administering a polypeptide to the subject in need thereof, wherein the polypeptide is X₁-ERTIPITRE-X₂ (SEQ ID NO:6), or a functional variant or mimetic thereof, wherein each X₁ and X₂ independently of one another represents any amino acid sequence of n amino acids, n varying from 0 to 50, and n being identical or different in X₁ and X₂, in an amount effective to reduce or eliminate the neoplastic disease in the subject.

10. The method of claim 9 wherein the functional variant or mimetic comprises a conservative amino acid substitution or peptide mimetic substitution.

11. The method of claim 9 wherein the functional variant comprises about 70% or greater amino acid sequence identity to X₁-ERTIPITRE-X₂ (SEQ ID NO:6).

12. The method of claim 9 wherein the functional variant has about 70% or greater amino acid sequence identity to X₁-RTIPITRE-X₂ (SEQ ID NO:7).

13. The method of claim 12 wherein the functional variant is X₁-RTIPITRE-X₂ (SEQ ID NO:7).

14. The method of claim 9 wherein the neoplastic disease is a solid tumor, carcinoma, sarcoma, lymphoma, or leukemia.

15. The method of claim 9 wherein the functional variant is a D-enantiomer of one or more amino acids.

16. The method of claim 9 wherein the functional variant is an L-enantiomer of one or more amino acids.

17. A method for treating a neoplastic disease in a mammalian subject comprising administering a polypeptide to the subject in need thereof, wherein the polypeptide is X₁-FISREFHR-X₂ (SEQ ID NO:8), or a functional variant or mimetic thereof, wherein each X₁ and X₂ independently of one another represents any amino acid sequence of n amino acids, n varying from 0 to 50, and n being identical or different in X₁ and X₂, in an amount effective to reduce or eliminate the neoplastic disease in the subject.

18. The method of claim 17 wherein the functional variant or mimetic comprises a conservative amino acid substitution or peptide mimetic substitution.

19. The method of claim 17 wherein the functional variant comprises about 70% or greater amino acid sequence identity to X₁-FISREFHR-X₂ (SEQ ID NO:8).

20. The method of claim 17 wherein the functional variant has about 70% or greater amino acid sequence identity to X₁-SREFHRKY-X₂ (SEQ ID NO:9), X₁-HGFISREF-X₂ (SEQ ID NO:10), or X₁-HGFISREFHRKYR-X₂ (SEQ ID NO:11).

21. The method of claim 18 wherein the functional variant is X₁-SREFHRKY-X₂ (SEQ ID NO:9), X₁-HGFISREF-X₂ (SEQ ID NO:10), or X₁-HGFISREFHRKYR-X₂ (SEQ ID NO:11).

22. The method of claim 17 wherein the neoplastic disease is a solid tumor, carcinoma, sarcoma, lymphoma, or leukemia.

23. The method of claim 17 wherein the functional variant is a D-enantiomer of one or more amino acids.

24. The method of claim 17 wherein the functional variant is an L-enantiomer of one or more amino acids.

25. A method for treating a neurodegenerative disease in a mammalian subject comprising administering a polypeptide to the subject in need thereof, wherein the polypeptide is X₁-LTITSSLSSDGV-X₂ (SEQ ID NO:4), or a functional variant or mimetic thereof, wherein each X₁ and X₂ independently of one another represents any amino acid sequence of n amino acids, n varying from 0 to 50, and n being identical or different in X₁ and X₂, or the polypeptide is X₃-ERTIPITRE-X₄ (SEQ ID NO:12), or a functional variant or mimetic thereof, wherein each X₃ and X₄ independently of one another represents any amino acid sequence of n amino acids, n varying from 0 to 50, and n being identical or different in X₃ and X₄, in an amount effective to reduce or eliminate the neurodegenerative disease in the subject.

26. The method of claim 25 wherein the functional variant or mimetic comprises a conservative amino acid substitution or peptide mimetic substitution.

27. The method of claim 25 wherein the functional variant comprises about 70% or greater amino acid sequence identity to X₁-LTITSSLSSDGV-X₂ (SEQ ID NO:4).

28. The method of claim 25 wherein the functional variant has about 70% or greater amino acid sequence identity to X₁-LTITSSLS-X₂ (SEQ ID NO:5).

29. The method of claim 28 wherein the functional variant is X₁-LTITSSLS-X₂ (SEQ ID NO:5).

30. The method of claim 25 wherein the functional variant comprises about 70% or greater amino acid sequence identity to X₃-ERTIPITRE-X₄ (SEQ ID NO:12).

31. The method of claim 25 wherein the functional variant has about 70% or greater amino acid sequence identity to X₃-RTIPITRE-X₄ (SEQ ID NO:33).

32. The method of claim 31 wherein the functional variant is X₃-RTIPITRE-X₄ (SEQ ID NO:33).

33. The method of claim 25 wherein the neurodegenerative disease is taupathy, Alzheimer's disease, motor neuron disease, hypoparathyroidism-retardation-dysmorphic syndrome, Parkinson's disease, schizophrenia, or Lewy body disease.

34. The method of claim 25 wherein the functional variant is a D-enantiomer of one or more amino acids.

35. The method of claim 25 wherein the functional variant is an L-enantiomer of one or more amino acids.

36. A method for inducing apoptosis of a cell in a mammalian subject comprising administering a polypeptide to the subject in need thereof, wherein the polypeptide is X₁-LTITSSLSSDGV-X₂ (SEQ ID NO:4), or a functional variant or mimetic thereof, wherein each X₁ and X₂ independently of one another represents any amino acid sequence of n amino acids, n varying from 0 to 50, and n being identical or different in X₁ and X₂, or the polypeptide is X₃-ERTIPITRE-X₄ (SEQ ID NO:12), or a functional variant or mimetic thereof, wherein each X₃ and X₄ independently of one another represents any amino acid sequence of n amino acids, n varying from 0 to 50, and n being identical or different in X₃ and X₄, in an amount effective to induce apoptosis and to reduce or eliminate a disease in the subject.

37. The method of claim 36 wherein the functional variant or mimetic comprises a conservative amino acid substitution or peptide mimetic substitution.

38. The method of claim 36 wherein the functional variant comprises about 70% or greater amino acid sequence identity to X₁-LTITSSLSSDGV-X₂ (SEQ ID NO:4).

39. The method of claim 36 wherein the functional variant has about 70% or greater amino acid sequence identity to X₁-LTITSSLS-X₂ (SEQ ID NO:5).

40. The method of claim 39 wherein the functional variant is X₁-LTITSSLS-X₂ (SEQ ID NO:5).

41. The method of claim 36 wherein the functional variant comprises about 70% or greater amino acid sequence identity to X₃-ERTIPITRE-X₄ (SEQ ID NO:12).

42. The method of claim 36 wherein the functional variant has about 70% or greater amino acid sequence identity to X₃-RTIPITRE-X₄ (SEQ ID NO:33).

43. The method of claim 42 wherein the functional variant is X₃-RTIPITRE-X₄ (SEQ ID NO:33).

44. The method of claim 36 wherein the disease is a neoplastic disease.

45. The method of claim 44 wherein the neoplastic disease is a solid tumor, carcinoma, sarcoma, lymphoma, or leukemia.

46. The method of claim 36 wherein the functional variant is a D-enantiomer of one or more amino acids.

47. The method of claim 36 wherein the functional variant is an L-enantiomer of one or more amino acids.

48. An in vivo method of screening for a modulator of microtubule assembly or disassembly activity comprising:
contacting a cell with a test compound encoding a polypeptide X₁-LTITSSLSSDGV-X₂ (SEQ ID NO:4), or a functional variant or mimetic thereof, wherein each X₁ and X₂ independently of one another represents any amino acid sequence of n amino acids, n varying from 0 to 50, and n being identical or different in X₁ and X₂, or a polypeptide X₃-ERTIPITRE-X₄ (SEQ ID NO:12), or a functional variant or mimetic thereof, wherein each X₃ and X₄ independently of one another represents any amino acid sequence of n amino acids, n varying from 0 to 50, and n being identical or different in X₃ and X₄, or the polypeptide is X₅-FISREFHR-X₆ (SEQ ID NO:18), or a functional variant or mimetic thereof, wherein each X₅ and X₆ independently of one another represents any amino acid sequence of n amino acids, n varying from 0 to 50, and n being identical or different in X₁ and X₂, and
detecting an interaction of the test compound with tubulin to promote microtubule assembly, inhibit microtubule disassembly, or decrease thermal aggregation of tubulin in the cell or cell line.

49. The method of claim 48 wherein the detecting step further comprises detecting apoptosis in the cell.

50. The method of claim 48 wherein the functional variant has about 70% or greater amino acid sequence identity to X₁-LTITSSLSSDGV-X₂ (SEQ ID NO:4).

51. The method of claim 48 wherein the functional variant has about 70% or greater amino acid sequence identity to X₁-LTITSSLS-X₂ (SEQ ID NO:5).

52. The method of claim 51 wherein the functional variant is X₁-LTITSSLS-X₂ (SEQ ID NO:5).

53. The method of claim 48 wherein the functional variant or mimetic comprises a conservative amino acid substitution or peptide mimetic substitution.

54. The method of claim 48 wherein the functional variant comprises about 70% or greater amino acid sequence identity to X₃-ERTIPITRE-X₄ (SEQ ID NO:12).

55. The method of claim 48 wherein the functional variant has about 70% or greater amino acid sequence identity to X₃-RTIPITRE-X₄ (SEQ ID NO:33).

56. The method of claim 55 wherein the functional variant is X₃-RTIPITRE-X₄ (SEQ ID NO:33).

57. The method of claim 48 wherein the functional variant is a D-enantiomer of one or more amino acids.
